(19) United States
(12) Patent Application Publication
(10) Pub. No.: US 2024/0182480 A1
(43) Pub. Date: Jun. 6, 2024

(54) 5-HT2A AND/OR 5-HT2C RECEPTOR AGONISTS

(71) Applicant: Diamond Therapeutics Inc., Toronto (CA)

(72) Inventor: Methvin ISAAC, Brampton (CA)

(21) Appl. No.: 18/501,238

(22) Filed: Nov. 3, 2023

Related U.S. Application Data
(63) Continuation of application No. PCT/IB2022/000254, filed on May 4, 2022.
(60) Provisional application No. 63/184,715, filed on May 5, 2021.

(51) Int. Cl. C07D 487/04; C07D 471/14; C07D 487/14

(52) U.S. Cl. CPC C07D 487/04; C07D 471/14; C07D 487/14

(57) ABSTRACT

Provided herein are heterocyclic compounds, processes for their preparation, compositions comprising said compounds, and use in therapy. More particularly, the present disclosure relates to fluorinated and/or deuterated analog useful in the treatment of diseases, disorders or conditions treatable by modulating the 5-HT2 receptor subtypes.

### CROSS-REFERENCE

This application is a continuation of International Application No. PCT/IB2022/000254, filed on May 4, 2022, which claims the benefit of U.S. Provisional Patent Application No. 63/184,715, filed May 5, 2021, which is incorporated herein by reference in its entirety.

### BACKGROUND

Provided herein are novel heterocyclic compounds, processes for their preparation, compositions comprising said compounds, and use in therapy. More particularly, the present disclosure relates to fluorinated and/or deuterated analog useful in the treatment of diseases, disorders or conditions treatable by modulating the 5-HT2 receptor subtypes.

### BRIEF SUMMARY OF THE INVENTION

Provided herein are heterocyclic 5-HT₂ₐ and/or 5-HT_{2c} receptor agonists compounds, pharmaceutical compositions comprising said compounds, and methods for using said compounds for the treatment of diseases.

One embodiment provides a compound, or pharmaceutically acceptable salt or solvate thereof, having the structure of Formula (I):

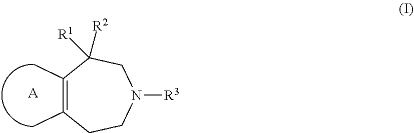

wherein,

R¹, R² and R³ are independently selected from H or C1-C6 optionally substituted alkyl; and Ring A is an optionally substituted heteroaryl ring selected from

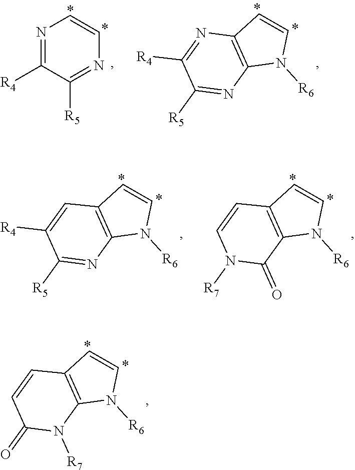

Wherein * represents the points of attachment, R⁴ and R⁵ are independently selected from H, halo, CN, C1-4alkyl, C1-4haloalkyl, OC1-4alkyl and OC1-4haloalkyl, NR⁸R⁹

R⁶ and R⁷ are independently selected from H or C1-C6 optionally substituted alkyl; and R⁸ and R⁹ are joined to form, together with the atom therebetween, C3-12heterocycloalkyl, optionally comprising one additional heteromoiety selected from NR10, O, S, S(O) and SO2, and optionally substituted with one or more substituents selected from halo, ═O, OH, C1-6alkyl, C3-6cycloalkyl, aryl, C5-6heteroaryl, C3-6heterocycloalkyl, C1-6alkyleneC3-6cycloalkyl, C1-6alkylenearyl, C1-6alkyleneC5-6heteroaryl, C1-6alkyleneC3-6heterocycloalkyl, C(O)C1-6alkyl, OC1-6alkyl, OC1-6alkyleneOC1-6alkyl, C(O)NH2, C(O)NH(C1-6alkyl), C(O)N(CT-6alkyl)(C1-6alkyl), NHC(O)C1-6alkyl, N(C1-6alkyl)C(O)C1-6alkyl, NH2, NH(C1-6alkyl), N(C1-6alkyl)(C1-6alkyl), SC1-6alkyl, S(O)C1-6alkyl and SO2C1-6alkyl, wherein all alkyl, alkylene, cycloalkyl, heterocycloalkyl, aryl and heteroaryl groups of the optional substituents on the C3-12heterocycloalkyl formed by R⁵ and R⁶ are also optionally substituted with one or more of halo, C1-6alkyl, OC1-6alkyl, C1-6haloalkyl and OC1-6haloalkyl;

One embodiment provides a pharmaceutical composition comprising a compound of Formula (I), or pharmaceutically acceptable salt or solvate thereof, and at least one pharmaceutically acceptable excipient.

In an aspect, the present disclosure provides a compound, or pharmaceutically acceptable salt or solvate thereof, having the structure of Formula (I):

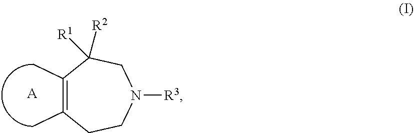

wherein
R¹, R² and R³ are each independently selected from H and C1-C6 optionally substituted alkyl; and
   ring A is an optionally substituted heteroaryl ring selected from

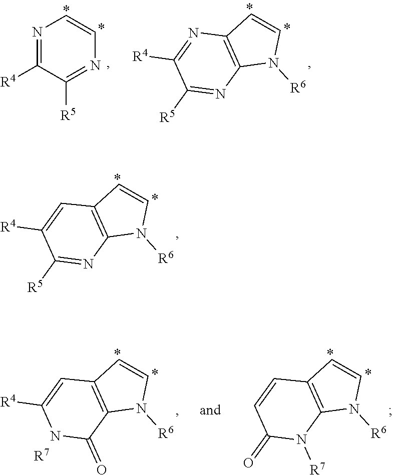

wherein
   * represents the points of attachment;
   R⁴ and R⁵ are independently selected from H, halo, CN, C₁₋₄alkyl, C₁₋₄haloalkyl, OC₁₋₄alkyl, OC₁₋₄haloalkyl, and N(R⁸R⁹);
   R⁶ and R⁷ are independently selected from H and C1-C6 optionally substituted alkyl; and
   R⁸ and R⁹ are joined to form, together with the atom therebetween, a C3-12heterocycloalkyl, optionally comprising one additional heteromoiety selected from N(R¹⁰), O, S, S(O) and SO₂, and optionally substituted with one or more substituents selected from halo, ═O, OH, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), N(C₁₋₆alkyl)(C₁₋₆alkyl), SC₁₋₆alkyl, S(O)C₁₋₆alkyl, and SO₂C₁₋₆alkyl,
   wherein
      R¹⁰ is selected from hydrogen, C₁₋₆alkyl, C₃₋₆cycloalkyl, aryl, C₅-6heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, C(O)NH₂, C(O)NH(C₁₋₆alkyl), C(O)N(C₁₋₆alkyl)(C₁₋₆alkyl), S(O)C₁₋₆alkyl, and SO₂C₁₋₆alkyl; and all alkyl, alkylene, cycloalkyl, heterocycloalkyl, aryl and heteroaryl groups of the optional substituents on the C₃₋₁₂heterocycloalkyl formed by R⁸ and R⁹ are optionally substituted with one or more substituents selected from halo, C₁₋₆alkyl, OC₁₋₆alkyl, C₁₋₆haloalkyl, and OC₁₋₆haloalkyl.

In some embodiments, R1 is H. In some embodiments, R1 is H, and R2 is C1-6 alkyl. In some embodiments, R1 is H, and R2 is methyl. In some embodiments, ring A is

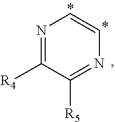

R1 is H, and R2 is C1-6 alkyl. In some embodiments, ring A

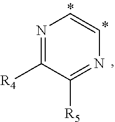

R1 is H, and R2 is methyl. In some embodiments, R3 is selected from H and C1-6 alkyl. In some embodiments, ring A is

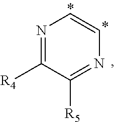

and R3 is H. In some embodiments, ring A is

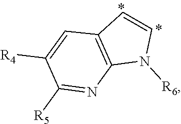

and R3 is selected from H and C1-6 alkyl. In some embodiments, ring A is

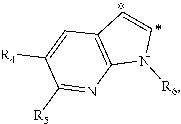

and R3 is selected from H and methyl. In some embodiments, ring A is

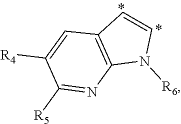

and R3 is methyl. In some embodiments, ring A is selected from

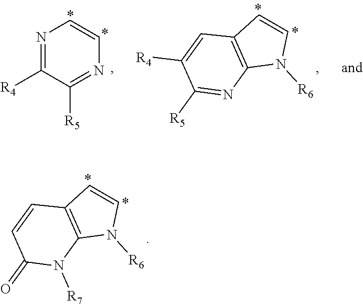

In some embodiments, ring A is selected from

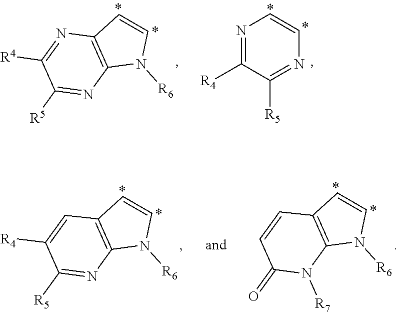

In some embodiments, ring A is

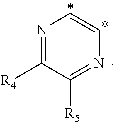

In some embodiments, ring A is

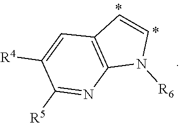

In some embodiments, ring A is

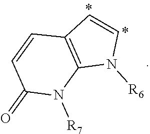

In some embodiments, ring A is

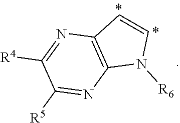

In some embodiments, ring A is

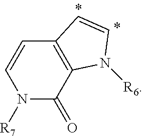

In some embodiments, ring A is

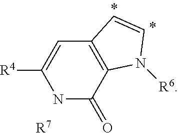

In some embodiments, R4 and R5 are independently selected from H, halo, CN, C1-4alkyl, C1-4haloalkyl, OC1-4alkyl, OC1-4haloalkyl, and N(R8R9). In some embodiments, R4 and R5 are independently selected from H, halo, C1-4alkyl, C1-4haloalkyl, OC1-4alkyl, OC1-4haloalkyl, and N(R8R9). In some embodiments, R4 and R5 are independently selected from H, halo, C1 alkyl, C₁-4haloalkyl, OC1-4alkyl, OC1-4haloalkyl, and N(R8R9). In some embodiments, R4 and R5 are independently selected from H, halo, C1-4haloalkyl, OC1-4alkyl, OC1-4haloalkyl, and N(R8R9). In some embodiments, R4 and R5 are independently selected from H and N(R8R9). In some embodiments, exactly one of R4 and R5 is H, and exactly one of R4 and R5 is N(R8R9). In some embodiments, exactly one of R4 and R5 is H, and exactly one of R4 and R5 is N(R8R9). In some embodiments, R4 is H, and R5 is N(R8R9). In some embodiments, ring A is

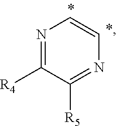

R4 is H, and R5 is N(R8R9). In some embodiments, ring A is

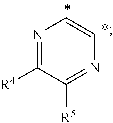

and R4 and R5 are each independently selected from H, NO2, F, CN, C1-4alkyl, C1-4haloalkyl, OC1-4alkyl, OC1-4haloalkyl, and N(R8R9). In some embodiments, ring A is

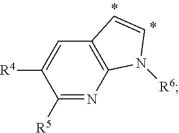

and R4 and R5 are each independently selected from H, NO2, F, CN, C2-4alkyl, C1-4haloalkyl, OC2-4alkyl, O(optionally substituted C3-10 carbocycle), O(optionally substituted C3-10 aryl), 0(3- to 10-membered heterocycle), O(3- to 10-membered heteroaryl), OC1-4haloalkyl, and N(R8R9). In some embodiments, R8 and R9 are joined to form, together with the atom therebetween, a C3-12heterocycloalkyl, optionally comprising one additional heteromoiety selected from N(R10), O, and S. In some embodiments, R8 and R9 are joined to form, together with the atom therebetween, a C3-12heterocycloalkyl, optionally comprising one additional heteromoiety selected from N(R10), and O. In some embodiments, R8 and R9 are joined to form, together with the atom therebetween, a C3-12heterocycloalkyl, optionally comprising one additional oxygen atom. In some embodiments, the C3-12heterocycloalkyl formed by R8 and R9 is optionally substituted with one or more substituents selected from halo, ═O, OH, C1-6alkyl, C3-6cycloalkyl, aryl, C5-6heteroaryl, C3-6 heterocycloalkyl, C1-6 alkyleneC₃₋₆cycloalkyl, C1-6alkylenearyl, C1-6alkyleneC5-6heteroaryl, C1-6alkyleneC₃₋₆heterocycloalkyl, C(O)C1-6alkyl, OC1-6alkyl, OC1-6alkyleneOC1-6alkyl, C(O)NH₂, C(O)NH(C1-6alkyl), C(O)N(C1-6alkyl)(C1-6alkyl), NHC(O)C1-6alkyl, N(C1-6alkyl)C(O)C1-6alkyl, NH₂, NH(C1-6alkyl), and N(C1-6alkyl)(C1-6alkyl). In some embodiments, the C3-12heterocycloalkyl formed by R8 and R9 is optionally substituted with one or more substituents selected from halo, ═O, OH, C1-6alkyl, C3-6cycloalkyl, aryl, C5-6heteroaryl, C3-6 heterocycloalkyl, C1-6 alkyleneC₃₋₆cycloalkyl, C1-6alkylenearyl, C1-6alkyleneC₅-6heteroaryl, C1-6alkyleneC3-6heterocycloalkyl, OC1-6alkyl, NH₂, NH(C1-6alkyl), and N(C1-6alkyl)(C1-6alkyl). In some embodiments, the C3-12heterocycloalkyl formed by R8 and R9 is optionally substituted with one or more substituents selected from halo, ═O, OH, OC1-6alkyl, NH₂, and NH(C1-6alkyl). In some embodiments, the C3-12heterocycloalkyl formed by R8 and R9 is optionally substituted with one or more substituents selected from halo, ═O, OH, OC1alkyl, NH₂, and NH(C1alkyl). In some embodiments, the C3-12heterocycloalkyl formed by R8 and R9 is optionally substituted with one or more substituents selected from halo, ═O, OH, OC1alkyl, NH₂, and NH(C1alkyl). In some embodiments, the C3-12heterocycloalkyl formed by R8 and R9 is optionally substituted with one or more substituents selected from fluoro, ═O, OH, OC1alkyl, NH₂, and NH(C1alkyl). In some embodiments, the C3-12heterocycloalkyl formed by R8 and R9 is optionally substituted with one or more fluoro substituents. In some embodiments, ring A is

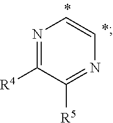

and the one additional heteromoiety of the C3-12heterocycloalkyl formed by R8 and R9 is NR10. In some embodiments, ring A is

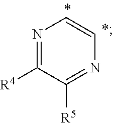

the one additional heteromoiety of the C3-12heterocycloalkyl formed by R8 and R9 is NR10; and the C3-12heterocycloalkyl formed by R8 and R9 is optionally substituted with one or more substituents selected from halo, OH, NO2, C1-6alkyl, C3-6cycloalkyl, aryl, C5-6heteroaryl, C3-6 heterocycloalkyl, C1-6 alkyleneC₃₋₆cycloalkyl, C1-6alkylenearyl, C1-6alkyleneC₅₋₆heteroaryl, C1-6alkyleneC₃₋₆heterocycloalkyl, C(O)C1-6alkyl, OC1-6alkyl, OC1-6alkyleneOC1-6alkyl, C(O)NH₂, C(O)NH(C1-6alkyl), C(O)N(C1-6alkyl)(C1-6alkyl), NHC(O)C1-6alkyl, N(C1-6alkyl)C(O)C1-6alkyl, NH₂, NH(C1-6alkyl), N(C1-6alkyl)(C1-6alkyl), SC1-6alkyl, S(O)C1-6alkyl, and SO₂C1-6alkyl. In some embodiments, ring A is

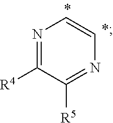

the one additional heteromoiety of the C3-12heterocycloalkyl formed by R8 and R9 is NR10; and the C3-12heterocycloalkyl formed by R8 and R9 is optionally substituted with one or more substituents selected from halo, OH, NO2, C1-6alkyl, C3-6cycloalkyl, aryl, C5-6heteroaryl, C3-6 heterocycloalkyl, and OC1-6alkyl. In some embodiments, ring A is

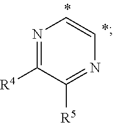

the one additional heteromoiety of the C3-12heterocycloalkyl formed by R8 and R9 is NR10; and R10 is selected from C1-6alkyl, C3-6cycloalkyl, aryl, C5-6heteroaryl, C3-6 heterocycloalkyl, C1-6 alkyleneC₃₋₆cycloalkyl, C1-6alkylenearyl, C1-6alkyleneC₅₋₆heteroaryl, C1-6alkyleneC₃₋₆heterocycloalkyl, C(O)C1-6alkyl, C(O)NH₂, C(O)NH(C1-6alkyl), C(O)N(C1-6alkyl)(C1-6alkyl), S(O)C1-6alkyl, and SO₂C1-6alkyl. In some embodiments, ring A is

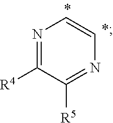

the one additional heteromoiety of the C3-12heterocycloalkyl formed by R8 and R9 is NR10; and R10 is selected from C1-6alkyl, C3-6cycloalkyl, aryl, C5-6heteroaryl, and C3-6 heterocycloalkyl. In some embodiments, ring A is

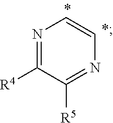

the one additional heteromoiety of the C3-12heterocycloalkyl formed by R8 and R9 is NR10; and R10 is selected from C1-6alkyl. In some embodiments, ring A is

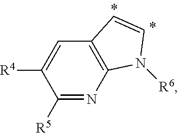

and the C3-12heterocycloalkyl formed by R8 and R9 is optionally substituted with one or more substituents selected from halo, NO2, OH, C1-6alkyl, C3-6cycloalkyl, aryl, C5-6heteroaryl, C3-6 heterocycloalkyl, C1-6 alkyleneC3-6cycloalkyl, C1-6alkylenearyl, C1-6alkyleneC5-6heteroaryl, C1-6alkyleneC₃₋₆heterocycloalkyl, C(O)C1-6alkyl, OC1-6alkyl, OC1-6alkyleneOC1-6alkyl, C(O)NH₂, C(O)NH(C1-6alkyl), C(O)N(C1-6alkyl)(C1-6alkyl), NHC(O)C1-6alkyl, N(C1-6alkyl)C(O)C1-6alkyl, NH₂, NH(C1-6alkyl), N(C1-6alkyl)(C1-6alkyl), SC1-6alkyl, S(O)C1-6alkyl, and SO₂C1-6alkyl. In some embodiments, ring A is

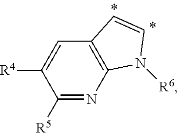

and the C3-12heterocycloalkyl formed by R8 and R9 is optionally substituted with one or more substituents selected from halo, NO2, OH, C1-6alkyl, C3-6cycloalkyl, aryl, C5-6heteroaryl, C3-6 heterocycloalkyl, C1-6 alkyleneC₃₋₆cycloalkyl, C1-6alkylenearyl, C1-6alkyleneC₅₋₆heteroaryl, C1-6alkyleneC₃₋₆heterocycloalkyl, C(O)C1-6alkyl, OC1-6alkyl, NH₂, and NH(C1-6alkyl). In some embodiments, ring A is

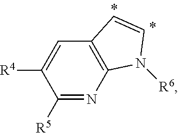

and the C3-12heterocycloalkyl formed by R8 and R9 is optionally substituted with one or more substituents selected from halo, NO2, OH, C1-6alkyl, C3-6cycloalkyl, aryl, C5-6heteroaryl, C3-6 heterocycloalkyl, C(O)C1-6alkyl, OC1-6alkyl, NH₂, and NH(C1-6alkyl). In some embodiments, ring A is

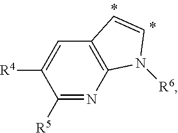

and the C3-12heterocycloalkyl formed by R8 and R9 is optionally substituted with one or more substituents selected from halo, NO2, OH, C1-6alkyl, OC1-6alkyl, NH₂, and NH(C1-6alkyl). In some embodiments, R10 is selected from hydrogen, C1-6alkyl, C3-6cycloalkyl, aryl, C5-6heteroaryl, C3-6 heterocycloalkyl, C1-6 alkyleneC₃₋₆cycloalkyl, C1-6alkylenearyl, C1-6alkyleneC₅-6heteroaryl, and C1-6alkyleneC3-6heterocycloalkyl. In some embodiments, R10 is selected from hydrogen, C1-6alkyl, C3-6cycloalkyl, aryl, C5-6heteroaryl, and C3-6 heterocycloalkyl. In some embodiments, R10 is selected from hydrogen, and C1-6alkyl. In some embodiments, the heterocycle formed by R8 and R9 is selected from

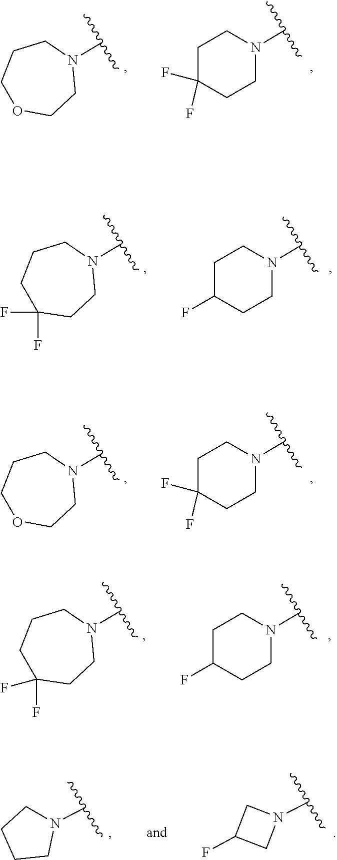

In some embodiments, R8 and R9 are methyl. In some embodiments, R1 and R2 are H; R3 is selected from H and C1-C6 optionally substituted alkyl; ring A is

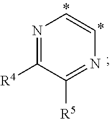

R4 and R5 are each independently selected from H, C1-4haloalkyl, OC1-4alkyl, OC1-4haloalkyl, O(optionally substituted C3-10 carbocycle), O(optionally substituted C3-10 aryl), and N(R8R9); R8 and R9 are joined to form, together with the atom therebetween, a C3-12heterocycloalkyl, and optionally substituted with one or more substituents selected from halo, OH, NO2, C1-6alkyl, C3-6cycloalkyl, aryl, C5-6heteroaryl, C3-6 heterocycloalkyl, C1-6 alkyleneC₃₋₆cycloalkyl, C1-6alkylenearyl, C1-6alkyleneC5-6heteroaryl, C1-6alkyleneC₃₋₆heterocycloalkyl, C(O)C1-6alkyl, OC1-6alkyl, OC1-6alkyleneOC1-6alkyl, C(O)NH₂, C(O)NH(C1-6alkyl), C(O)N(C1-6alkyl)(C1-6alkyl), NHC(O)C1-6alkyl, N(C1-6alkyl)C(O)C1-6alkyl, NH₂, NH(C1-6alkyl), N(C1-6alkyl)(C1-6alkyl), SC1-6alkyl, S(O)C1-6alkyl, and SO₂C1-6alkyl, wherein R10 is selected from C1-6alkyl, C3-6cycloalkyl, aryl, C5-6heteroaryl, C3-6 heterocycloalkyl, C1-6 alkyleneC₃₋₆cycloalkyl, C1-6alkylenearyl, C1-6alkyleneC₅-6heteroaryl, C1-6alkyleneC3-6heterocycloalkyl, C(O)C1-6alkyl, C(O)NH₂, C(O)NH(C1-6alkyl), C(O)N(C1-6alkyl)(C1-6alkyl), S(O)C1-6alkyl, and SO₂C1-6alkyl. In some embodiments, R1 and R2 are H; R3 is selected from H and C1-C6 optionally substituted alkyl; ring A is

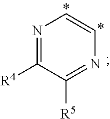

R4 and R5 are each independently selected from H, C1-4haloalkyl, OC1-4alkyl, OC1-4haloalkyl, O(optionally substituted C3-10 carbocycle), O(optionally substituted C3-10 aryl), and N(R8R9);
R8 and R9 are joined to form, together with the atom therebetween, a C3-12heterocycloalkyl, optionally comprising one additional heteromoiety selected from N(R10) and O and optionally substituted with one or more substituents selected from halo, OH, NO2, C1-6alkyl, C3-6cycloalkyl, aryl, C5-6heteroaryl, C3-6 heterocycloalkyl, C(O)C1-6alkyl, OC1-6alkyl, NH₂, and NH(C1-6alkyl), wherein R10 is selected from C1-6alkyl, C3-6cycloalkyl, aryl, C5-6heteroaryl, C3-6 heterocycloalkyl, C(O)C1-6alkyl, C(O)NH₂, and C(O)NH(C1-6alkyl). In some embodiments, R1 and R2 are H; R3 is selected from H and C1-C6 optionally substituted alkyl; ring A is

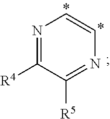

R4 and R5 are each independently selected from H, C1-4haloalkyl, OC1-4alkyl, and N(R8R9);
R8 and R9 are joined to form, together with the atom therebetween, a C3-12heterocycloalkyl, optionally comprising one additional oxygen atom and optionally substituted with one or more substituents selected from halo, OH, NO2, C1-6alkyl, aryl, C5-6heteroaryl, C3-6 heterocycloalkyl, OC1-6alkyl, NH₂, and NH(C1-6alkyl). In some embodiments, R1 and R2 are H; R3 is selected from H and C1-C6 optionally substituted alkyl; ring A is

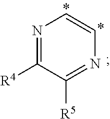

R4 and R5 are each independently selected from H, C1-4haloalkyl, OC1-4alkyl, and N(R8R9); R8 and R9 are joined to form, together with the atom therebetween, a C3-12heterocycloalkyl, optionally comprising one additional oxygen atom and optionally substituted with one or more substituents selected from halo, OH, NO2, C1-6alkyl, aryl, and C5-6heteroaryl. In some embodiments, R¹ and R2 are H; R3 is selected from H and C1-C6 optionally substituted alkyl; ring A is

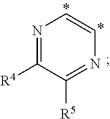

R4 and R5 are each independently selected from H and N(R8R9);
R8 and R9 are joined to form, together with the atom therebetween, a C3-12heterocycloalkyl, optionally comprising one additional oxygen atom and optionally substituted with one or more substituents selected from halo, OH, NO2, C1-6alkyl, aryl, and C5-6heteroaryl. In some embodiments, R1 and R2 are H; R3 is selected from H and C1-C6 optionally substituted alkyl; ring A is

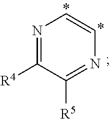

R4 and R5 are each independently selected from H and N(R8R9); R8 and R9 are joined to form, together with the atom therebetween, a C3-12heterocycloalkyl, optionally comprising one additional oxygen atom and optionally substituted with one or more substituents selected from halo, OH, and C1-6alkyl. In some embodiments, R1 and R2 are H; R3 is selected from H and C1-C6 optionally substituted alkyl; ring A is

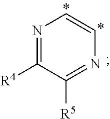

R4 and R5 are each independently selected from H and N(R8R9);

R8 and R9 are joined to form, together with the atom therebetween, a C3-12heterocycloalkyl, optionally comprising one additional oxygen atom and optionally substituted with one or more substituents selected from halo, and OH. In some embodiments, R1 and R2 are H; R3 is selected from H and C1-C6 optionally substituted alkyl; ring A is

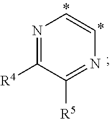

R4 and R5 are each independently selected from H and N(R8R9); R8 and R9 are joined to form, together with the atom therebetween, a C3-12heterocycloalkyl, optionally comprising one additional oxygen atom and optionally substituted with one or more halo substituents. In some embodiments, R1 and R2 are H; R3 is selected from H and C1-C6 optionally substituted alkyl; ring A is

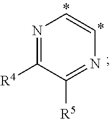

R4 and R5 are each independently selected from H and N(R8R9);

R8 and R9 are joined to form, together with the atom therebetween, a C3-12heterocycloalkyl, optionally comprising one additional oxygen atom and optionally substituted with one or more fluoro substituents. In some embodiments, R1 and R2 are H; R3 is selected from H and C1-C6 optionally substituted alkyl; ring A is

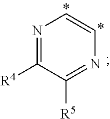

R4 and R5 are each independently selected from H, C1-4haloalkyl, OC1-4alkyl, OC1-4haloalkyl, O(optionally substituted C3-10 carbocycle), O(optionally substituted C3-10 aryl), and N(R8R9); R8 and R9 are joined to form, together with the atom therebetween, a C3-12heterocycloalkyl, optionally comprising one additional oxygen atom and optionally substituted with one or more substituents selected from halo, OH, NO2, C1-6alkyl, C3-6cycloalkyl, aryl, C5-6heteroaryl, C3-6 heterocycloalkyl, C(O)C1-6alkyl, OC1-6alkyl, OC1-6alkyleneOC1-6alkyl, NH₂, and NH(C1-6alkyl).

In an aspect, the present disclosure provides a compound, or pharmaceutically acceptable salt or solvate thereof, having the structure of Formula (I):

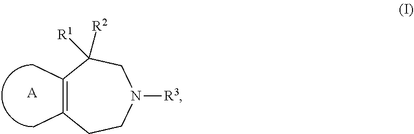

wherein
   R¹, R² and R³ are each independently selected from H and C1-C6 optionally substituted alkyl; and
   ring A is an optionally substituted heteroaryl ring selected from

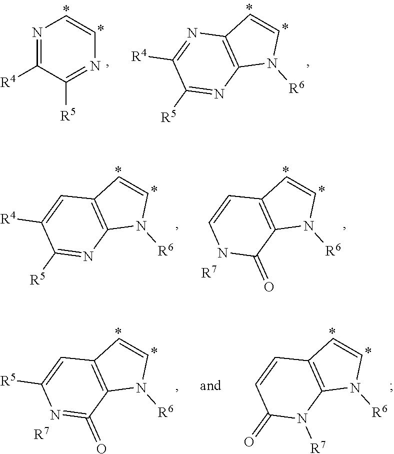

wherein
   * represents the points of attachment;
   R⁴ and R⁵ are each independently selected from H, NO₂, halo, CN, C₁₋₄ alkyl, C₁₋₄haloalkyl, OC₁₋₄alkyl, O(optionally substituted C₃₋₁₀ carbocycle), O(optionally substituted C₃₋₁₀aryl), O(3- to 10-membered heterocycle), O(3- to 10-membered heteroaryl), OC₁₋₄haloalkyl, and N(R⁸R⁹), wherein
      each OC₁₋₄alkyl, OC₃₋₁₀aryl, and O(3- to 10-membered heteroaryl), of R⁴ and R⁵ is independently optionally substituted with one or more substituents selected from C₃₋₆ carbocycle, C₃₋₆ aryl, 3- to 10-membered heterocycle, and 3- to 10-membered heteroaryl, wherein each C₃₋₆ carbocycle, C₃₋₆ aryl, 3- to 10-membered heterocycle, and 3- to 10-membered heteroaryl of R⁴ and R⁵, is optionally substituted with one or more substituents selected from halogen, C₁₋₆ alkyl, C₁₋₆aminoalkyl, CN, NO₂, OH, and C₁₋₆ alkoxy;
      when ring A is

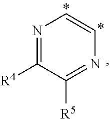

R⁴ and R⁵ are each independently selected from H, NO₂, F, CN, C₁₋₄alkyl, C₁₋₄haloalkyl, OC1-4alkyl, OC1-4haloalkyl, O(optionally substituted C₃₋₁₀ carbocycle), O(optionally substituted C₃₋₁₀ aryl), O(3- to 10-membered heterocycle), O(3- to 10-membered heteroaryl), and N(R⁸R⁹); and
when ring A is

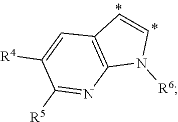

R⁴ and R⁵ are each independently selected from H, NO₂, F, CN, C₂₋₄alkyl, C₁₋₄haloalkyl, OC2-4alkyl, O(optionally substituted carbocycle), O(optionally substituted heterocycle), OC₁₋₄haloalkyl, and N(R⁸R⁹);
R⁶ and R⁷ are each independently selected from H and C₁-C₆ optionally substituted alkyl; and
R⁸ and R⁹ are each independently selected from
   hydrogen;
   C₁₋₆ optionally substituted alkyl, C(O)(C₁₋₆ optionally substituted alkyl), C(O)N(C₁₋₆ optionally substituted alkyl)₂, C(O)O(C₁₋₆ optionally substituted alkyl), S(O)(C₁₋₆ optionally substituted alkyl), and S(O)₂(C₁₋₆ optionally substituted alkyl), wherein
   when ring A is

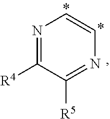

and R⁸ is H; R⁹ is selected from C(O)N(C₁₋₆ optionally substituted alkyl)₂, C(O)O(C₁₋₆ optionally substituted alkyl), S(O)(C₁₋₆ optionally substituted alkyl), and S(O)₂(C₁₋₆ optionally substituted alkyl); and
C₃₋₁₀ carbocycle, C₃₋₁₀ aryl, 3- to 10-membered heteroaryl, and 3- to 10-membered heterocycle, optionally substituted with one or more substituents selected from halo, C₁₋₆alkyl, OC₁₋₆alkyl, C₁₋₆haloalkyl, and OC₁₋₆haloalkyl; and C₁₋₆ alkyl, optionally substituted with one or more substituents selected from halo, C₁₋₆alkyl, OC₁₋₆alkyl, C₁₋₆haloalkyl, and OC₁₋₆haloalkyl; and C₃₋₁₀ carbocycle, C₃₋₁₀ aryl, 3- to 10-membered heteroaryl, and 3- to 10-membered heterocycle, optionally substituted with one or more substituents selected from halo, C₁₋₆alkyl, OC₁₋₆alkyl, C₁₋₆haloalkyl, and OC₁₋₆haloalkyl; or
R⁸ and R⁹ are joined to form, together with the atom therebetween, a C₃₋₁₂heterocycloalkyl, optionally substituted with one or more substituents selected from NO2, CN, halo, ═O, OH, C₁₋₆alkyl, C₃₋₆cycloalkyl, aryl, C₅-6heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), N(C₁₋₆alkyl)(C₁₋₆alkyl), SC₁₋₆alkyl, S(O)C₁₋₆alkyl, and SO₂C₁₋₆alkyl, wherein
R¹⁰ is selected from hydrogen, C₁₋₆alkyl, C₃₋₆cycloalkyl, aryl, C₅-6heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃-6heterocycloalkyl, C(O)C₁₋₆alkyl, C(O)NH₂, C(O)NH(C₁₋₆alkyl), C(O)N(C₁₋₆alkyl)(C₁₋₆alkyl), S(O)C₁₋₆alkyl, and SO₂C₁₋₆alkyl; all alkyl, alkylene, cycloalkyl, heterocycloalkyl, aryl and heteroaryl groups of the optional substituents on the C₃₋₁₂heterocycloalkyl formed by R⁸ and R⁹ are optionally substituted with one or more substituents selected from halo, C₁₋₆alkyl, OC₁₋₆alkyl, C₁₋₆haloalkyl, and OC₁₋₆haloalkyl;
when ring A is

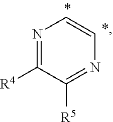

and the one additional heteromoiety of the C₃₋₁₂heterocycloalkyl formed by R⁸ and R⁹ is NR¹⁰;
the C₃₋₁₂heterocycloalkyl formed by R⁸ and R⁹ is optionally substituted with one or more substituents selected from halo, OH, NO2, C₁₋₆alkyl, C₃₋₆cycloalkyl, aryl, C₅₋₆heteroaryl, C₃₋₆ heterocycloalkyl, C₁₋₆ alkyleneC₃-6cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), N(C₁₋₆alkyl)(C₁₋₆alkyl), SC₁₋₆alkyl, S(O)C₁₋₆alkyl, and SO₂C₁₋₆alkyl; and
R¹⁰ is selected from C₁₋₆alkyl, C₃₋₆cycloalkyl, aryl, C₅-6heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, C(O)NH₂, C(O)NH(C₁₋₆alkyl), C(O)N(C₁₋₆alkyl)(C₁₋₆alkyl), S(O)C₁₋₆alkyl, and SO₂C₁₋₆alkyl; and
when ring A is

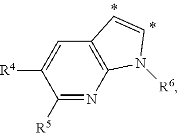

the C₃₋₁₂heterocycloalkyl formed by R⁸ and R⁹ is optionally substituted with one or more substituents selected from halo, NO₂, OH, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), N(C₁₋₆alkyl)(C₁₋₆alkyl), SC₁₋₆alkyl, S(O)C₁₋₆alkyl, and SO₂C₁₋₆alkyl.

In some embodiments, R¹ is H. In some embodiments, R¹ is H, and R² is C₁₋₆ embodiments, R¹ is H, and R² is methyl. In some embodiments, ring A is

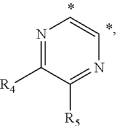

R¹ is H, and R² is C₁₋₆ allkyl. In some embodiments, ring A is

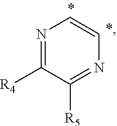

R¹ is H, and R² is methyl. In some embodiments, R³ is selected from H and C₁₋₆ alkyl. In some embodiments, R³ is selected from H. In some embodiments, ring A is

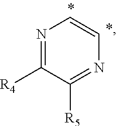

and R³ is H. In some embodiments, ring A is

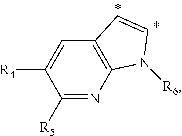

and R³ is selected from H and C₁₋₆ alkyl. In some embodiments, ring A is

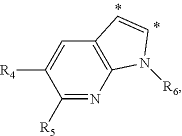

and R³ is selected from H and methyl. In some embodiments, ring A is

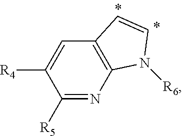

and R³ is methyl. In some embodiments, ring A is selected from

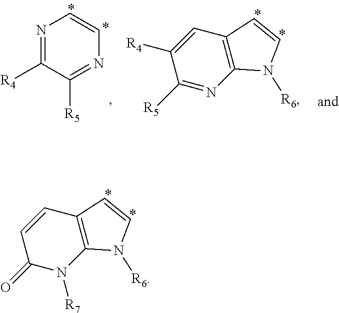

In some embodiments, ring A is selected from

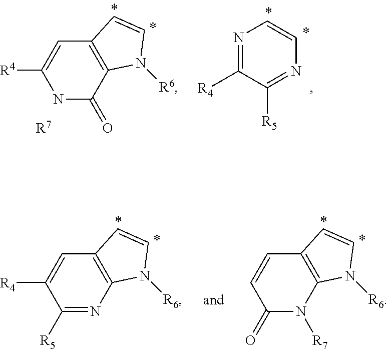

In some embodiments, ring A is

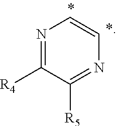

In some embodiments, ring A is

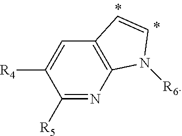

In some embodiments, ring A is

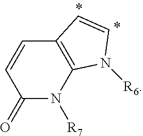

In some embodiments, ring A is

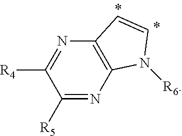

In some embodiments, ring A is

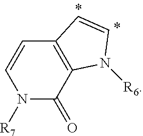

In some embodiments, ring A is

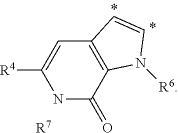

In some embodiments, R⁴ and R⁵ are independently selected from H, halo, CN, C₁₋₄ alkyl, C₁₋₄haloalkyl, OC₁₋₄alkyl, O(optionally substituted C₃₋₁₀ carbocycle), O(optionally substituted C₃₋₁₀ aryl), O(3- to 10-membered heterocycle), O(3- to 10-membered heteroaryl), OC₁₋₄haloalkyl, and N(R⁸R⁹), In some embodiments, R⁴ and R⁵ are independently selected from H, halo, CN, C₁₋₄ alkyl, C₁₋₄haloalkyl, OC₁₋₄alkyl, O(optionally substituted C₃₋₁₀ carbocycle), O(optionally substituted C₃₋₁₀ aryl), OC₁₋₄haloalkyl, and N(R⁸R⁹). In some embodiments, R⁴ and R⁵ are independently selected from H, halo, C₁₋₄haloalkyl, OC₁₋₄alkyl, O(optionally substituted C₃₋₁₀ carbocycle), O(optionally substituted C₃₋₁₀ aryl), OC₁₋₄haloalkyl, and N(R⁸R⁹). In some embodiments, R⁴ and R⁵ are independently selected from H and N(R⁸R⁹). In some embodiments, exactly one of R⁴ and R⁵ is H, and exactly one of R⁴ and R⁵ is N(R⁸R⁹). In some embodiments, exactly one of R⁴ and R⁵ is H, and exactly one of R⁴ and R⁵ is N(R⁸R⁹). In some embodiments, R⁴ is H, and R⁵ is N(R⁸R⁹). In some embodiments, ring A is

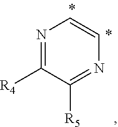

R⁴ is H, and R⁵ is N(R⁸R⁹). In some embodiments, ring A is

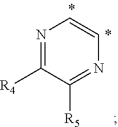

and R⁴ and R⁵ are each independently selected from H, NO₂, halo, CN, C₁₋₄ alkyl, C₁₋₄haloalkyl, OC₁₋₄alkyl, O(optionally substituted C₃₋₁₀ carbocycle), O(optionally substituted C₃₋₁₀ aryl), O(3- to 10-membered heterocycle), O(3 to 10-membered heteroaryl), OC1-4haloalkyl, and N(R⁸R⁹). In some embodiments, ring A is

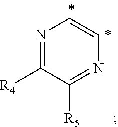

and R⁴ and R⁵ are each independently selected from H, NO₂, halo, CN, C₁₋₄ alkyl, C₁₋₄haloalkyl, OC₁₋₄alkyl, O(optionally substituted C₃₋₁₀ carbocycle), O(optionally substituted C₃₋₁₀ aryl), and OC₁₋₄haloalkyl, and N(R⁸R⁹). In some embodiments, ring A is

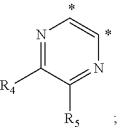

and R⁴ and R⁵ are each independently selected from H, halo, C₁₋₄ alkyl, C₁₋₄haloalkyl, OC₁₋₄alkyl, O(optionally substituted C₃₋₁₀ carbocycle), O(optionally substituted C₃₋₁₀ aryl), and OC₁₋₄haloalkyl, and N(R⁸R⁹). In some embodiments, ring A is

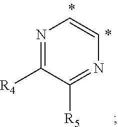

and R⁴ and R⁵ are each independently selected from H, halo, C₁₋₄haloalkyl, OC₁₋₄alkyl, O(optionally substituted C₃₋₁₀ carbocycle), O(optionally substituted C₃₋₁₀ aryl), and OC₁₋₄haloalkyl, and N(R⁸R⁹). In some embodiments, R⁸ and R⁹ are joined to form, together with the atom therebetween, a C₃₋₁₂heterocycloalkyl, optionally comprising one additional heteromoiety selected from N(R¹⁰), O, and S. In some embodiments, R⁸ and R⁹ are joined to form, together with the atom therebetween, a C₃₋₁₂heterocycloalkyl, optionally comprising one additional heteromoiety selected from N(R¹⁰), and O. In some embodiments, R⁸ and R⁹ are joined to form, together with the atom therebetween, a C₃₋₁₂heterocycloalkyl, optionally comprising one additional oxygen atom. In some embodiments, the C₃₋₁₂heterocycloalkyl formed by R⁸ and R⁹ is optionally substituted with one or more substituents selected from NO₂, CN, halo, ═O, OH, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), and N(C₁₋₆alkyl)(C₁₋₆alkyl). In some embodiments, the C₃₋₁₂heterocycloalkyl formed by R⁸ and R⁹ is optionally substituted with one or more substituents selected from NO₂, CN, halo, ═O, OH, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, OC1-6alkyl, NH₂, NH(C₁₋₆alkyl), and N(C₁₋₆alkyl)(C₁₋₆alkyl). In some embodiments, the C₃₋₁₂heterocycloalkyl formed by R⁸ and R⁹ is optionally substituted with one or more substituents selected from NO₂, CN, halo, ═O, OH, OC₁₋₆alkyl, NH₂, and NH(C₁₋₆alkyl). In some embodiments, the C₃₋₁₂heterocycloalkyl formed by R⁸ and R⁹ is optionally substituted with one or more substituents selected from NO₂, CN, halo, ═O, OH, OC₁alkyl, NH₂, and NH(C₁alkyl). In some embodiments, the C₃₋₁₂heterocycloalkyl formed by R⁸ and R⁹ is optionally substituted with one or more substituents selected from NO₂, CN, halo, ═O, OH, OC₁alkyl, NH₂, and NH(C₁alkyl). In some embodiments, the C₃₋₁₂heterocycloalkyl formed by R⁸ and R⁹ is optionally substituted with one or more substituents selected from NO₂, CN, fluoro, ═O, OH, OC₁alkyl, NH₂, and NH(C₁alkyl). In some embodiments, the C₃₋₁₂heterocycloalkyl formed by R⁸ and R⁹ is optionally substituted with one or more substituents selected from fluoro, ═O, OH, OC₁alkyl, NH₂, and NH(C₁alkyl). In some embodiments, the C₃₋₁₂heterocycloalkyl formed by R⁸ and R⁹ is optionally substituted with one or more fluoro substituents. In some embodiments, ring A is

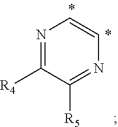

and the one additional heteromoiety of the C₃₋₁₂heterocycloalkyl formed by R⁸ and R⁹ is NR¹⁰. In some embodiments, ring A is

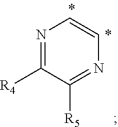

the one additional heteromoiety of the C₃₋₁₂heterocycloalkyl formed by R⁸ and R⁹ is NR¹⁰; and the C₃₋₁₂heterocycloalkyl formed by R⁸ and R⁹ is optionally substituted with one or more substituents selected from halo, OH, NO₂, C₁₋₆alkyl, C₃₋₆cycloalkyl, aryl, C₅-6heteroaryl, C₃₋₆ heterocycloalkyl, C₁₋₆ alkyleneC₃₋₆cycloalkyl, C₁₋₆alkylenearyl, C₁₋₆alkyleneC₅-6heteroaryl, C₁₋₆alkyleneC₃₋₆heterocycloalkyl, C(O)C₁₋₆alkyl, OC1-6alkyl, OC1-6alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), N(C₁₋₆alkyl)(C₁₋₆alkyl), SC₁₋₆alkyl, S(O)C₁₋₆alkyl, and SO₂C₁₋₆alkyl. In some embodiments, ring A is

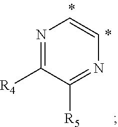

the one additional heteromoiety of the C₃₋₁₂heterocycloalkyl formed by R⁸ and R⁹ is NR¹⁰; and the C₃₋₁₂heterocycloalkyl formed by R⁸ and R⁹ is optionally substituted with one or more substituents selected from halo, OH, NO₂, C₁₋₆alkyl, C₃₋₆cycloalkyl, aryl, C₅₋₆heteroaryl, C₃₋₆ heterocycloalkyl, and OC1-6alkyl. In some embodiments, ring A is

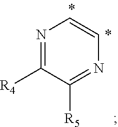

the one additional heteromoiety of the C₃₋₁₂heterocycloalkyl formed by R⁸ and R⁹ is NR¹⁰; and R¹⁰ is selected from C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, C(O)NH₂, C(O)NH(C₁₋₆alkyl), C(O)N(C₁₋₆alkyl)(C₁₋₆alkyl), S(O)C₁₋₆alkyl, and SO₂C₁₋₆alkyl. In some embodiments, ring A is

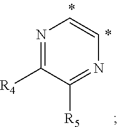

the one additional heteromoiety of the C₃₋₁₂heterocycloalkyl formed by R⁸ and R⁹ is NR¹⁰; and R¹⁰ is selected from C₁₋₆alkyl, C₃₋₆cycloalkyl, aryl, C₅₋₆heteroaryl, and C₃₋₆ heterocycloalkyl. In some embodiments, ring A is

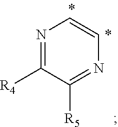

the one additional heteromoiety of the C₃₋₁₂heterocycloalkyl formed by R⁸ and R⁹ is NR¹⁰; and R¹⁰ is selected from C₁₋₆alkyl. In some embodiments, ring A is

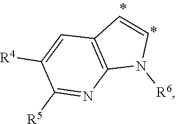

and the C₃₋₁₂heterocycloalkyl formed by R⁸ and R⁹ is optionally substituted with one or more substituents selected from halo, NO₂, OH, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), N(C₁₋₆alkyl)(C₁₋₆alkyl), SC₁₋₆alkyl, S(O)C₁₋₆alkyl, and SO₂C₁₋₆alkyl. In some embodiments, ring A is

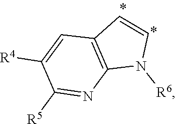

and the C₃₋₁₂heterocycloalkyl formed by R⁸ and R⁹ is optionally substituted with one or more substituents selected from halo, NO₂, OH, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, NH₂, and NH(C₁₋₆alkyl). In some embodiments, ring A is

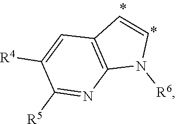

and the C₃₋₁₂heterocycloalkyl formed by R⁸ and R⁹ is optionally substituted with one or more substituents selected from halo, NO₂, OH, C₁₋₆alkyl, C₃₋₆cycloalkyl, aryl, C₅₋₆heteroaryl, C₃₋₆ heterocycloalkyl, C(O)C₁₋₆alkyl, OC₁₋₆alkyl, NH₂, and NH(C₁₋₆alkyl). In some embodiments, ring A is

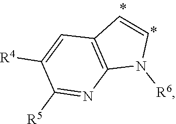

and the C₃₋₁₂heterocycloalkyl formed by R⁸ and R⁹ is optionally substituted with one or more substituents selected from halo, NO₂, OH, C₁₋₆alkyl, OC₁₋₆alkyl, NH₂, and NH(C₁₋₆alkyl). In some embodiments, R¹⁰ is selected from hydrogen, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, and C₁₋₆alkyleneC₃₋₆heterocycloalkyl. In some embodiments, R¹⁰ is selected from hydrogen, C₁₋₆alkyl, C₃₋₆cycloalkyl, aryl, C₅₋₆heteroaryl, and C₃₋₆ heterocycloalkyl. In some embodiments, R¹⁰ is selected from hydrogen, and C₁₋₆alkyl. In some embodiments, the heterocycle formed by R⁸ and R⁹ is selected from

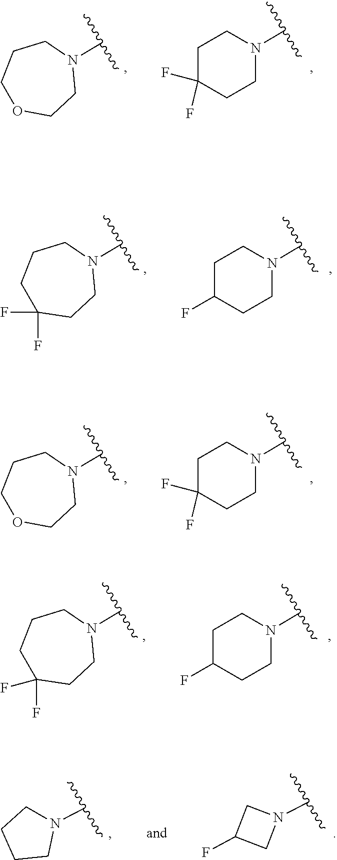

In some embodiments, R⁸ and R⁹ are methyl. In some embodiments, R¹ and R² are H; R³ is selected from H and C₁-C₆ optionally substituted alkyl; ring A is

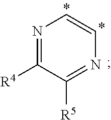

R⁴ and R⁵ are each independently selected from H, C₁₋₄haloalkyl, OC₁₋₄alkyl, OC₁₋₄haloalkyl, O(optionally substituted C₃₋₁₀ carbocycle), O(optionally substituted C₃₋₁₀ aryl), and N(R⁸R⁹);
R⁸ and R⁹ are joined to form, together with the atom therebetween, a C₃₋₁₂heterocycloalkyl, optionally comprising one additional heteromoiety selected from N(R¹⁰), O, S, S(O) and SO₂, and optionally substituted with one or more substituents selected from halo, OH, NO₂, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), N(C₁₋₆alkyl)(C₁₋₆alkyl), SC₁₋₆alkyl, S(O)C₁₋₆alkyl, and SO₂C₁₋₆alkyl, wherein R¹⁰ is selected from C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, C(O)NH₂, C(O)NH(C₁₋₆alkyl), C(O)N(C₁₋₆alkyl)(C₁₋₆alkyl), S(O)C₁₋₆alkyl, and SO₂C₁₋₆alkyl. In some embodiments, R¹ and R² are H; R³ is selected from H and C₁-C₆ optionally substituted alkyl; ring A is

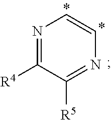

R⁴ and R⁵ are each independently selected from H, C₁₋₄haloalkyl, OC₁₋₄alkyl, OC₁₋₄haloalkyl, O(optionally substituted C₃₋₁₀ carbocycle), O(optionally substituted C₃₋₁₀ aryl), and N(R⁸R⁹);
R⁸ and R⁹ are joined to form, together with the atom therebetween, a C₃₋₁₂heterocycloalkyl, optionally comprising one additional heteromoiety selected from N(R¹⁰) and O and optionally substituted with one or more substituents selected from halo, OH, NO₂, C₁₋₆alkyl, C₃₋₆cycloalkyl, aryl, C₅₋₆heteroaryl, C₃₋₆ heterocycloalkyl, C(O)C₁₋₆alkyl, OC₁₋₆alkyl, NH₂, and NH(C₁₋₆alkyl), wherein R¹⁰ is selected from C₁₋₆alkyl, C₃₋₆cycloalkyl, aryl, C₅₋₆heteroaryl, C₃₋₆ heterocycloalkyl, C(O)C₁₋₆alkyl, C(O)NH₂, and C(O)NH(C₁₋₆alkyl). In some embodiments, R¹ and R² are H;
R³ is selected from H and C₁-C₆ optionally substituted alkyl; ring A is

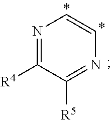

R⁴ and R⁵ are each independently selected from H, C₁₋₄haloalkyl, OC1-4alkyl, and N(R⁸R⁹);
R⁸ and R⁹ are joined to form, together with the atom therebetween, a C₃₋₁₂heterocycloalkyl, optionally comprising one additional oxygen atom and optionally substituted with one or more substituents selected from halo, OH, NO₂, C₁₋₆alkyl, aryl, C₅₋₆heteroaryl, C₃₋₆ heterocycloalkyl, OC₁₋₆alkyl, NH₂, and NH(C₁₋₆alkyl). In some embodiments, R¹ and R² are H; R³ is selected from H and C₁-C₆ optionally substituted alkyl; ring A is

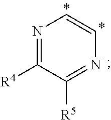

R⁴ and R⁵ are each independently selected from H, C₁₋₄haloalkyl, OC₁₋₄alkyl, and N(R⁸R⁹);
R⁸ and R⁹ are joined to form, together with the atom therebetween, a C₃₋₁₂heterocycloalkyl, optionally comprising one additional oxygen atom and optionally substituted with one or more substituents selected from halo, OH, NO₂, C₁₋₆alkyl, aryl, and C₅₋₆heteroaryl. In some embodiments, R¹ and R² are H; R³ is selected from H and C₁-C₆ optionally substituted alkyl; ring A is

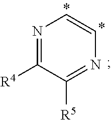

R⁴ and R⁵ are each independently selected from H and N(R⁸R⁹); R⁸ and R⁹ are joined to form, together with the atom therebetween, a C₃₋₁₂heterocycloalkyl, optionally comprising one additional oxygen atom and optionally substituted with one or more substituents selected from halo, OH, NO₂, C₁₋₆alkyl, aryl, and C₅₋₆heteroaryl. In some embodiments, R¹ and R² are H; R³ is selected from H and C₁-C₆ optionally substituted alkyl; ring A is

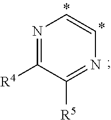

R⁴ and R⁵ are each independently selected from H and N(R⁸R⁹); R⁸ and R⁹ are joined to form, together with the atom therebetween, a C₃₋₁₂heterocycloalkyl, optionally comprising one additional oxygen atom and optionally substituted with one or more substituents selected from halo, OH, and C₁₋₆alkyl. In some embodiments, R¹ and R² are H;
R³ is selected from H and C₁-C₆ optionally substituted alkyl; ring A is

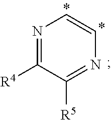

R⁴ and R⁵ are each independently selected from H and N(R⁸R⁹); R⁸ and R⁹ are joined to form, together with the atom therebetween, a C₃₋₁₂heterocycloalkyl, optionally comprising one additional oxygen atom and optionally substituted with one or more substituents selected from halo, and OH. In some embodiments, R¹ and R² are H; R³ is selected from H and C₁-C₆ optionally substituted alkyl; ring A is

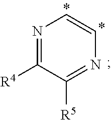

R⁴ and R⁵ are each independently selected from H and N(R⁸R⁹),
R⁸ and R⁹ are joined to form, together with the atom therebetween, a C₃₋₁₂heterocycloalkyl, optionally comprising one additional oxygen atom and optionally substituted with one or more halo substituents. In some embodiments, R¹ and R² are H; R³ is selected from H and C₁-C₆ optionally substituted alkyl; ring A is

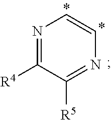

R⁴ and R⁵ are each independently selected from H and N(R⁸R⁹); R⁸ and R⁹ are joined to form, together with the atom therebetween, a C₃₋₁₂heterocycloalkyl, optionally comprising one additional oxygen atom and optionally substituted with one or more fluoro substituents. In some embodiments, R¹ and R² are H; R³ is selected from H and C₁-C₆ optionally substituted alkyl; ring A is

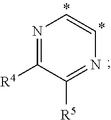

R⁴ and R⁵ are each independently selected from H, C₁₋₄haloalkyl, OC₁₋₄alkyl, OC₁₋₄haloalkyl, O(optionally substituted C₃₋₁₀ carbocycle), O(optionally substituted C₃₋₁₀ aryl), and N(R⁸R⁹); R⁸ and R⁹ are joined to form, together with the atom therebetween, a C₃₋₁₂heterocycloalkyl, optionally comprising one additional oxygen atom and optionally substituted with one or more substituents selected from halo, OH, NO₂, C₁₋₆alkyl, C₃₋₆cycloalkyl, aryl, C₅₋₆heteroaryl, C₃₋₆ heterocycloalkyl, C(O)C₁₋₆alkyl, OC₁₋₆alkyl, OC₁₋₆alkyleneOC₁₋₆alkyl, NH₂, and NH(C₁₋₆alkyl). In some embodiments, the compound, pharmaceutically acceptable salt or solvate thereof, has the structure:

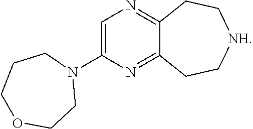 [SMILES: c1nc2c(nc1N1CCCOCC1)CCNCC2]

In some embodiments, the compound, pharmaceutically acceptable salt or solvate thereof, has the structure:

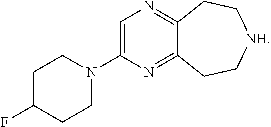 [SMILES: FC1CCN(c2cnc3c(n2)CCNCC3)CC1]

In some embodiments, the compound, pharmaceutically acceptable salt or solvate thereof, has the structure:

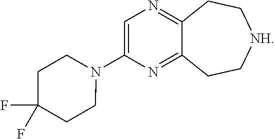 [SMILES: FC1(F)CCN(c2cnc3c(n2)CCNCC3)CC1]

In some embodiments, the compound, pharmaceutically acceptable salt or solvate thereof, has the structure: F

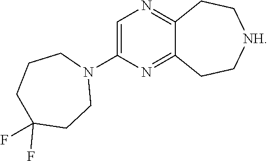 [SMILES: FC1(F)CCCN(c2cnc3c(n2)CCNCC3)CC1]

In an aspect, the present disclosure provides a compound, or pharmaceutically acceptable salt or solvate thereof, as provided in Table 1.

In an aspect, the present disclosure provides a compound, or pharmaceutically acceptable salt or solvate thereof, selected from

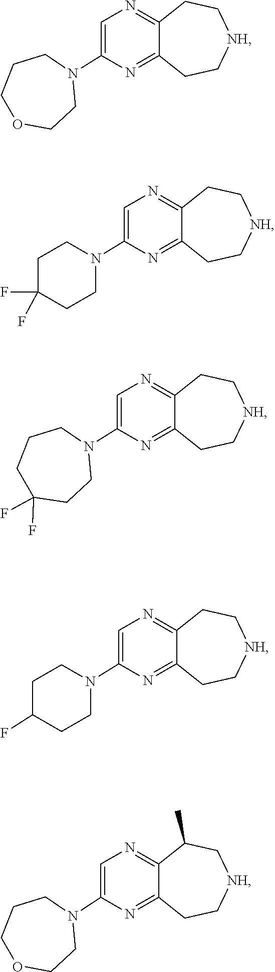 [SMILES: c1nc2c(nc1N1CCCOCC1)CCNCC2 ; FC1(F)CCN(c2cnc3c(n2)CCNCC3)CC1 ; FC1(F)CCCN(c2cnc3c(n2)CCNCC3)CC1 ; FC1CCN(c2cnc3c(n2)CCNCC3)CC1 ; C[C@H]1CNCCc2nc(N3CCCOCC3)cnc21]

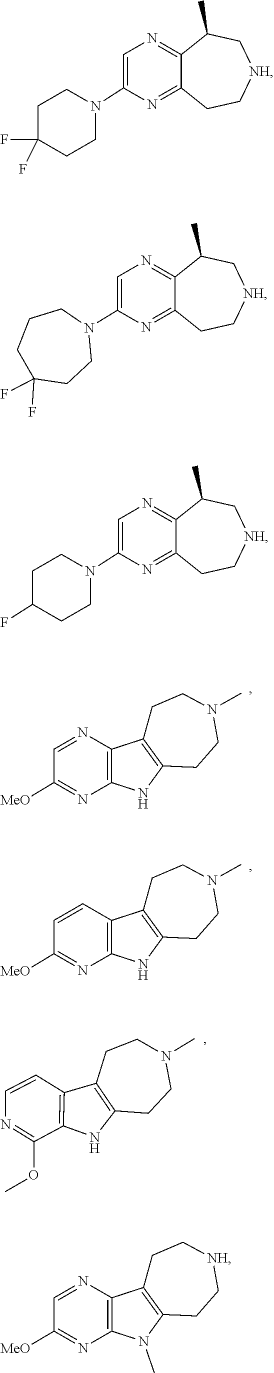

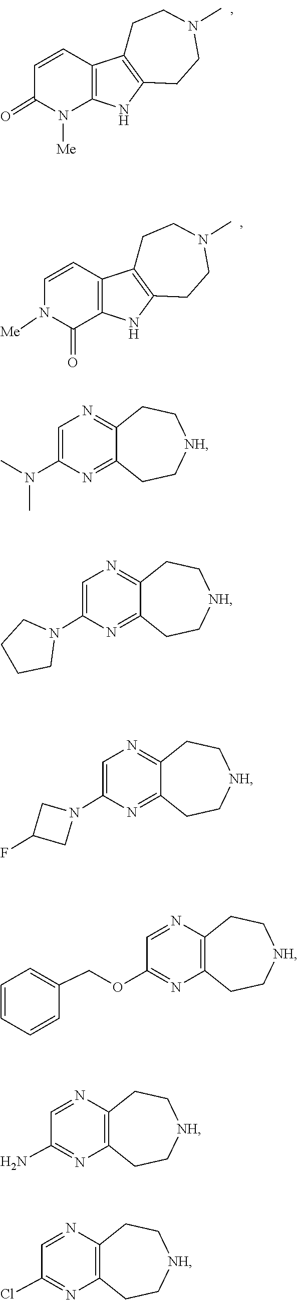

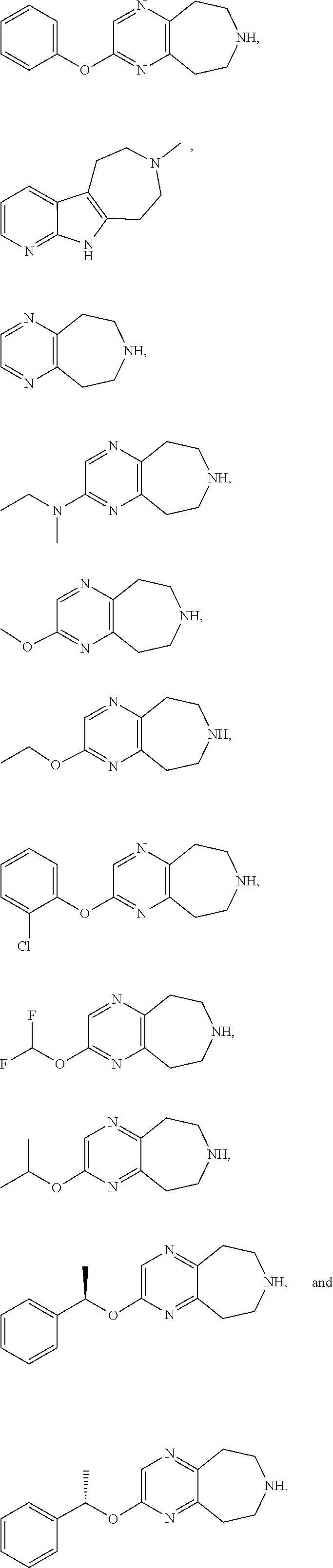

In an aspect, the present disclosure provides a pharmaceutical composition comprising a compound, or pharmaceutically acceptable salt or solvate thereof, as described in any of the embodiments disclosed herein and a pharmaceutically acceptable excipient. In an aspect, the present disclosure provides a pharmaceutical composition comprising a compound, or pharmaceutically acceptable salt or solvate thereof, as described in any of the embodiments disclosed herein and a pharmaceutically acceptable excipient. In an aspect, the present disclosure provides use of the composition of any of the embodiments disclosed herein in the treatment of a disease or disorder mediated by the 5-HT2 receptor. In an aspect, the present disclosure provides use of the compound, salt, or solvate of any of the embodiments disclosed herein in the treatment of a disease or disorder mediated by the 5-HT2 receptor. In an aspect, the present disclosure provides the use of any of the embodiments disclosed herein according to any of the embodiments disclosed herein, wherein the disease or disorder is a 5-HT_{2A} and/or 5-HT2e receptor-mediated disorder. The use according to any of the embodiments disclosed herein, wherein the disease or disorder is depressive disorder, an anxiety disorder, panic attack, agoraphobia, specific phobia, social phobia, bipolar disorder, post-traumatic stress, an eating disorder, obesity, a gastro-intestinal disorder, alcoholism, drug addiction, schizophrenia, a psychotic disorder, a sleep disorder, sleep apnea, migraine, sexual dysfunction, a central nervous system disorder, trauma, stroke, spinal cord injury, a cardio-vascular disorder, diabetes insipidus, or obsessive disorder.

In an aspect, the present disclosure provides a use of the composition of any of the embodiments disclosed herein to ameliorate at least one symptom of a brain disorder, stress, anxiety, addiction, depression, compulsive behavior, or by promoting weight loss, or by improving mood, or by treating or preventing a psychological disorder, or by enhancing performance. In an aspect, the present disclose provides a method of treating at least one symptom of a brain disorder, stress, anxiety, addiction, depression, or compulsive behavior comprising administering to a patient in need thereof the compound, salt, or solvate of any of the embodiments disclosed herein.

In an aspect, the present disclosure provides a method of promoting weight loss comprising administering to a patient in need thereof the compound, salt, or solvate of any of the embodiments disclosed herein. In an aspect, the present disclosure provides a method of improving mood comprising administering to a patient in need thereof the compound, salt, or solvate of any of the embodiments disclosed herein.

In an aspect, the present disclosure provides a method of preventing a psychological disorder comprising administering to a patient in need thereof the compound, salt, or solvate of any of the embodiments disclosed herein.

In an aspect, the present disclosure provides a method of enhancing performance comprising administering to a patient in need thereof the compound, salt, or solvate of any of the embodiments disclosed herein.

In an aspect, the present disclosure provides a method of treating depressive disorder, an anxiety disorder, panic attack, agoraphobia, specific phobia, social phobia, bipolar disorder, post-traumatic stress, an eating disorder, obesity, a gastro-intestinal disorder, alcoholism, drug addiction, schizophrenia, a psychotic disorder, a sleep disorder, sleep apnea, migraine, sexual dysfunction, a central nervous system disorder, trauma, stroke, spinal cord injury, a cardio-vascular disorder, diabetes insipidus, or obsessive disorder comprising administering to a patient in need thereof the compound, salt, or solvate of any of the embodiments disclosed herein.

In an aspect, the present disclosure provides a method of preparing the compound

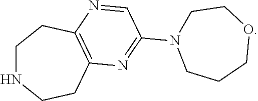 [SMILES: c1nc2c(nc1N1CCCOCC1)CCNCC2]

In an aspect, the present disclosure provides a method of preparing the compound

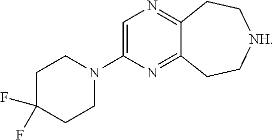 [SMILES: FC1(F)CCN(c2cnc3c(n2)CCNCC3)CC1]

In an aspect, the present disclosure provides a method of preparing the compound

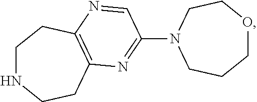 [SMILES: c1nc2c(nc1N1CCCOCC1)CCNCC2]

the method comprising: (a) a chlorination of an aminopyrazine, optionally comprising treatment of the aminopyrizine with tBuONO or TiCl₄ to form a chloropyrazine; (b) a nucleophilic aromatic substitution of the chloropyrazine, optionally comprising treatment with a primary or secondary amine and optionally comprising treatment with a base, to form an aminopyrazine.

In an aspect, the present disclosure provides method of preparing the compound

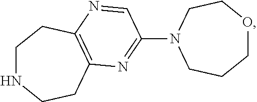 [SMILES: c1nc2c(nc1N1CCCOCC1)CCNCC2]

the method comprising: (a) a chlorination of an aminopyrazine, optionally comprising treatment of the aminopyrizine with tBuONO or TiCl₄ to form a chloropyrazine; (b) a nucleophilic aromatic substitution of the chloropyrazine, optionally comprising treatment with a primary or secondary amine and optionally comprising treatment with a base, to form an aminopyrazine; and further comprising (c) a deprotection of a protected amine, optionally comprising an N-dealkylation comprising treatment with 1-chloroethyl chloroformate.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.
“Amino” refers to the —NH₂ radical.
“Cyano” refers to the —CN radical.
“Nitro” refers to the —NO₂ radical.
“Oxa” refers to the —O— radical.
“Oxo” refers to the ═O radical.
“Thioxo” refers to the ═S radical.
“Imino” refers to the ═N—H radical.
“Oximo” refers to the ═N—OH radical.
“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₅ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₅ alkenylene). In other embodiments, an alkenylene comprises three to five carbon atoms (e.g., C₃-C₅ alkenylene).

Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₅ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₅ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hickel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical is optionally substituted as defined above.

“Carbocyclylalkynyl” refers to a radical of the formula —R^{c}-carbocyclyl where R^{c} is an alkynylene chain as defined above. The alkynylene chain and the carbocyclyl radical is optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical is optionally substituted as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

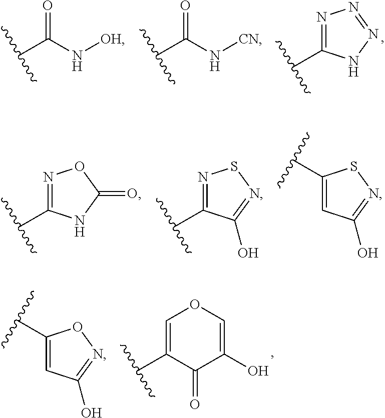

and the like.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hickel theory. Heteroaryl includes fused or bridged ring systems. The heteroatom(s) in the heteroaryl radical is optionally oxidized. One or more nitrogen atoms, if present, are optionally quatemized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-TH-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibria include:

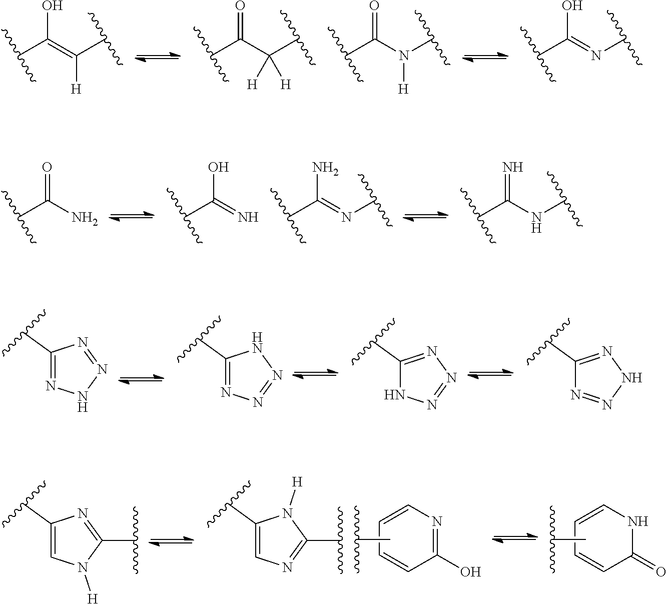

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. In some embodiments, isotopic substitution with ¹⁸F is contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

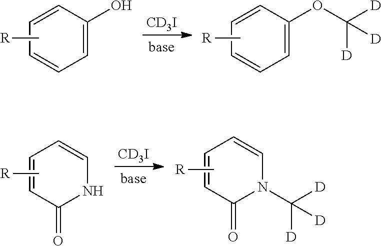

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

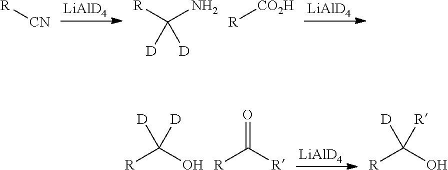

Deuterium gas and palladium catalyst are employed to reduce unsaturated carbon-carbon linkages and to perform a reductive substitution of aryl carbon-halogen bonds as illustrated, by way of example only, in the reaction schemes below.

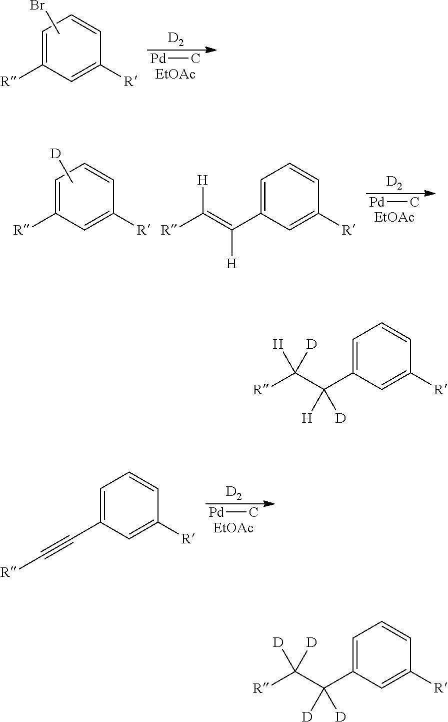

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the heterocyclic 5-HT₂ₐ and/or 5-HT_{2c} receptor agonists compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein optionally exist in either unsolvated as well as solvated forms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

Heterocyclic 5-HT₂ₐ and/or 5-HT₂ₑ Receptor Agonists Compounds

Neuropsychiatric diseases, including mood and anxiety disorders, are some of the leading causes of disability worldwide and place an enormous economic burden on society. Approximately one third of patients will not respond to current antidepressant drugs, and those who do will usually require at least two to four weeks of treatment before they experience any beneficial effects. Evidence from a combination of human imaging, postmortem studies, and animal models suggest that atrophy of neurons in the prefrontal cortex (PFC) plays a key role in the pathophysiology of depression and related disorders. These structural changes, such as the retraction of neurites and loss of dendritic spines, can potentially be counteracted by compounds capable of promoting structural and functional neural plasticity. Recently the nonclassical psychedelics has shown remarkable clinical potential as a fast-acting antidepressant and anxiolytic, exhibiting efficacy in treatment-resistant populations. Animal models suggest that its therapeutic effects stem from its ability to promote the growth of dendritic spines, increase the synthesis of synaptic proteins, and strengthen synaptic responses.

Clinical studies have demonstrated the potential for using classical psychedelics to treat a variety of neuropsychiatric disorders including depression, anxiety, addiction, and post-traumatic disorders. However, their therapeutic mechanism of action remains poorly understood, and concerns about safety have severely limited their clinical usefulness.

Psychedelic compounds have the potential to meet the therapeutic needs for a number of indications without the addictiveness and overdose risk of other mind-altering drugs, such as cocaine, heroin, alcohol, methamphetamine, and so forth. The need for new therapies is urgent because addiction, overdose, and suicide deaths have risen throughout the North America and around the world. The problem is further exacerbated by the lack of significant advances in psychiatric drug development, as current treatments are plagued with limited efficacy, significant side effects, and dependency on long time use, which may lead some patients to develop treatment-resistance. Recent academic research effort along with anecdotal reports suggest that psychedelics have promising therapeutic potential (BMC Psychiatry 2018, 18, 245).

Psychedelic compound research has previously been stymied as a result of governmental regulation and societal taboo which has left many unanswered questions regarding the pharmacology and toxicology of psychedelics. There has been renewed interest in the therapeutic potential of psychedelics. For example, psilocybin-assisted psychotherapy has been effective in the treatment of depression and anxiety in cancer patients and also in the treatment of resistant depression (J. Psychopharmacol. 2016, 30, 1181).

Therefore, the future of therapeutic psychedelics research in general holds enormous potential to save lives and meet unmet medical needs throughout the world.

The molecular features that could confer good metabolic and pharmacokinetic characteristic are unpredictable. We have identified key structural feature in compounds of Formula I that offer improved metabolic properties for the treatment of diseases, disorders or conditions treatable by activating the 5HT_{2A} and/or 5-HT_{2c} signaling axis.

In one aspect, provided herein is a heterocyclic 5-HT₂ₐ and/or 5-HT_{2c} receptor agonist compound.

One embodiment provides a compound, or pharmaceutically acceptable salt or solvate thereof, having the structure of Formula (I):

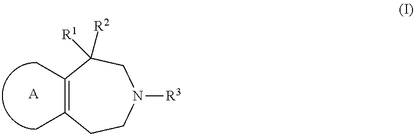

wherein,

R¹, R² and R³ are independently selected from H or C1-C6 optionally substituted alkyl; and Ring A is an optionally substituted heteroaryl ring selected from

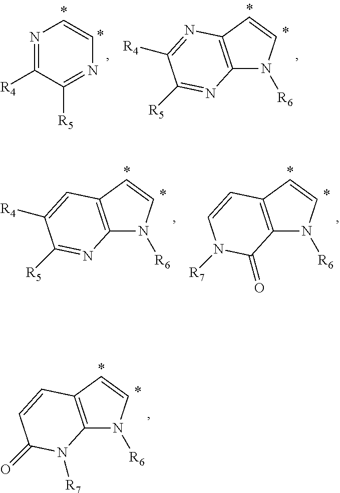

wherein * represents the points of attachment, R⁴ and R⁵ are independently selected from H, halo, CN, C1-4alkyl, C1-4haloalkyl, OC1-4alkyl and OC1-4haloalkyl, NR⁸R⁹

R⁶ and R⁷ are independently selected from H or C1-C6 optionally substituted alkyl; and

R⁸ and R⁹ are joined to form, together with the atom therebetween, C3-12heterocycloalkyl, optionally comprising one additional heteromoiety selected from NR¹⁰, O, S, S(O) and SO₂, and optionally substituted with one or more substituents selected from halo, ═O, OH, C1-6alkyl, C3-6cycloalkyl, aryl, C5-6heteroaryl, C3-6heterocycloalkyl, C1-6alkyleneC₃₋₆cycloalkyl, C1-6alkylenearyl, C1-6alkyleneC₅₋₆heteroaryl, C1-6alkyleneC₃₋₆heterocycloalkyl, C(O)C1-6alkyl, OC₁₋₆alkyl, OC1-6alkyleneOC1-6alkyl, C(O)NH₂, C(O)NH(C1-6alkyl), C(O)N(C1-6alkyl)(C1-6alkyl), NHC(O)C1-6alkyl, N(C1-6alkyl)C(O)C1-6alkyl, NH₂, NH(C1-6alkyl), N(C1-6alkyl)(C1-6alkyl), SC1-6alkyl, S(O)C1-6alkyl and SO₂C1-6alkyl, wherein all alkyl, alkylene, cycloalkyl, heterocycloalkyl, aryl and heteroaryl groups of the optional substituents on the C3-12heterocycloalkyl formed by R⁵ and R⁶ are also optionally substituted with one or more of halo, C1-6alkyl, OC₁₋₆alkyl, C1-6haloalkyl and OC₁₋₆haloalkyl.

In an aspect, ring A can be attached to the core (e.g., azepane core, e.g., tetrahydroazepine core) of formula (I) in a first possible orientation or a second possible orienation. For example, in some embodiments, ring A is

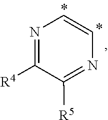

the first possible orientation is

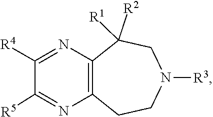

and the second possible orientation is

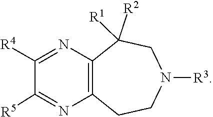

In some embodiments, ring A is

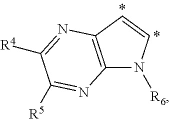

the first possible orientation is

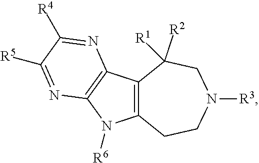

and the second possible orientation is

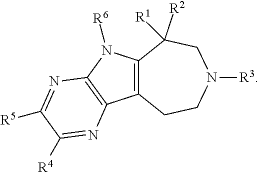

In some embodiments, ring A is

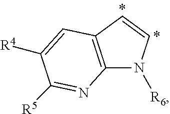

the first possible orientation is

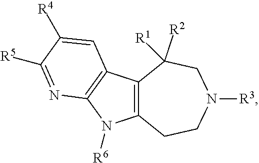

and the second possible orientation is

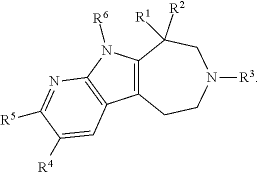

In some embodiments, ring A is

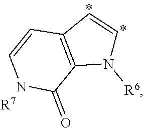

the first possible orientation is

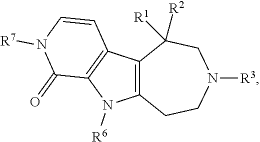

and the second possible orientation is

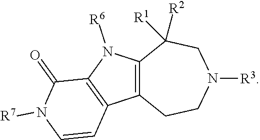

In some embodiments, ring A is

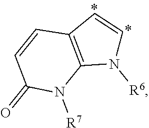

the first possible orientation is

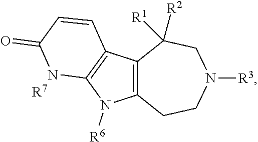

and the second possible orientation is

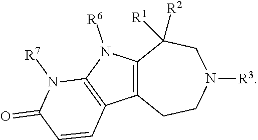

In some embodiments, the heterocyclic 5-HT₂ₐ and/or 5-HT_{2c} receptor agonist compound as described herein has a structure and/or compound name provided in Table 1.

TABLE 1

| Synthetic | | |
|---|---|---|
| Chemistry | | |
| Example | Compound Structure | Compound Name |
| 1 | 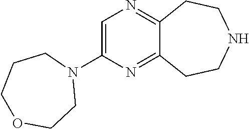 [SMILES: c1nc2c(nc1N1CCCOCC1)CCNCC2] | 4-(6,7,8,9-tetrahydro-5H-pyrazino[2,3- d]azepin-2-yl)-1,4-oxazepane (e.g., 4- {5H,6H,7H,8H,9H-pyrazino[2,3- d]azepin-2-yl}-1,4-oxazepane) |
| 2 | 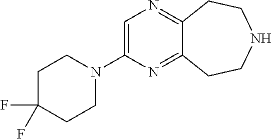 [SMILES: FC1(F)CCN(c2cnc3c(n2)CCNCC3)CC1] | 2-(4,4-difluoropiperidin-1-yl)-6,7,8,9- tetrahydro-5H-pyrazino[2,3-d]azepine |
| 3 | 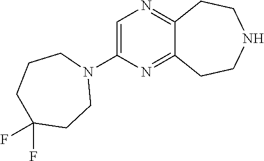 [SMILES: FC1(F)CCCN(c2cnc3c(n2)CCNCC3)CC1] | 2-(4,4-difluoroazepan-1-yl)-6,7,8,9- tetrahydro-5H-pyrazino[2,3-d]azepine |
| 4 | 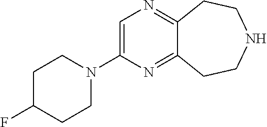 [SMILES: FC1CCN(c2cnc3c(n2)CCNCC3)CC1] | 2-(4-fluoropiperidin-1-yl)-6,7,8,9- tetrahydro-5H-pyrazino[2,3-d]azepine |
| 5 | 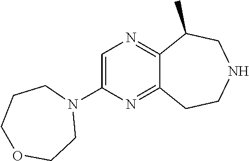 [SMILES: C[C@H]1CNCCc2nc(N3CCCOCC3)cnc21] | (S)-4-(5-methyl-6,7,8,9-tetrahydro-5H- pyrazino[2,3-d]azepin-2-yl)-1,4- oxazepane |
| 6 | 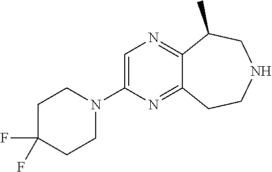 [SMILES: C[C@H]1CNCCc2nc(N3CCC(F)(F)CC3)cnc21] | (S)-2-(4,4-difluoropiperidin-1-yl)-5- methyl-6,7,8,9-tetrahydro-5H- pyrazino[2,3-d]azepine |
| 7 | 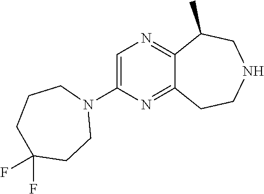 [SMILES: C[C@H]1CNCCc2nc(N3CCCC(F)(F)CC3)cnc21] | (S)-2-(4,4-difluoroazepan-1-yl)-5- methyl-6,7,8,9-tetrahydro-5H- pyrazino[2,3-d]azepine |
| 8 | 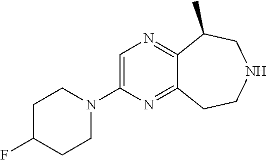 [SMILES: C[C@H]1CNCCc2nc(N3CCC(F)CC3)cnc21] | (S)-2-(4-fluoropiperidin-1-yl)-5-methyl- 6,7,8,9-tetrahydro-5H-pyrazino[2,3- d]azepine |
| 9 | 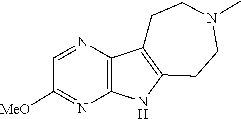 [SMILES: COc1cnc2c3c([nH]c2n1)CCN(C)CC3] | 3-methoxy-8-methyl-5,6,7,8,9,10- hexahydropyrazino[2′,3′:4,5]pyrrolo[2,3- d]azepine |
| 10 | 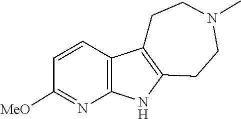 [SMILES: COc1ccc2c3c([nH]c2n1)CCN(C)CC3] | 2-methoxy-7-methyl-5,6,7,8,9,10- hexahydropyrido[3′,2′:4,5]pyrrolo[2,3- d]azepine |
| 11 | 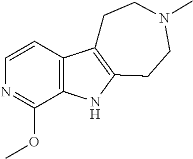 [SMILES: COc1nccc2c3c([nH]c12)CCN(C)CC3] | 1-methoxy-7-methyl-5,6,7,8,9,10- hexahydropyrido[4′,3′:4,5]pyrrolo[2,3- d]azepine |
| 12 | 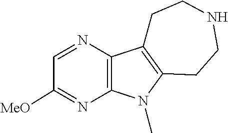 [SMILES: COc1cnc2c3c(n(C)c2n1)CCNCC3] | 3-methoxy-5-methyl-5,6,7,8,9,10- hexahydropyrazino[2′,3′:4,5]pyrrolo[2,3- d]azepine |
| 13 | 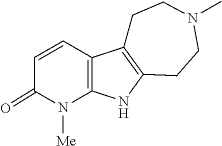 [SMILES: CN1CCc2[nH]c3c(ccc(=O)n3C)c2CC1] | 1,7-dimethyl-5,6,7,8,9,10- hexahydropyrido[3′,2′:4,5]pyrrolo[2,3- d]azepin-2(1H)-one |
| 14 | 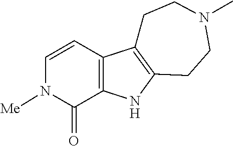 [SMILES: CN1CCc2[nH]c3c(=O)n(C)ccc3c2CC1] | 2,7-dimethyl-5,6,7,8,10- hexahydropyrido[4′,3′:4,5]pyrrolo[2,3- d]azepin-1(2H)-one |
| 15 | 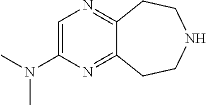 | N,N-dimethyl-6,7,8,9-tetrahydro-5H- pyrazino[2,3-d]azepin-2-amine |
| 16 | 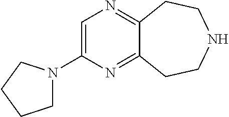 [SMILES: c1nc2c(nc1N1CCCC1)CCNCC2] | 2-(pyrrolidin-1-yl)-6,7,8,9-tetrahydrahydro- 5H-pyrazino[2,3-d]azepine |
| 17 | 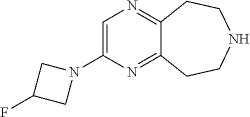 [SMILES: FC1CN(c2cnc3c(n2)CCNCC3)C1] | 2-(3-fluoroazetidin-1-yl)-6,7,8,9- tetrahydro-5H-pyrazino[2,3-d]azepine |
| 18 | 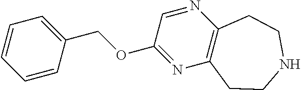 [SMILES: c1ccc(COc2cnc3c(n2)CCNCC3)cc1] | 2-(benzyloxy)-6,7,8,9-tetrahydro-5H- pyrazino[2,3-d]azepine |
| 19 | 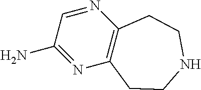 [SMILES: Nc1cnc2c(n1)CCNCC2] | 6,7,8,9-tetrahydro-5H-pyrazino[2,3- d]azepin-2-amine |
| 20 | 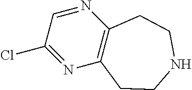 [SMILES: Clc1cnc2c(n1)CCNCC2] | 2-chloro-6,7,8,9-tetrahydro-5H- pyrazino[2,3-d]azepine |
| 21 | 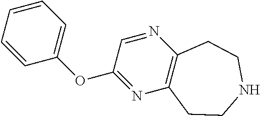 [SMILES: c1ccc(Oc2cnc3c(n2)CCNCC3)cc1] | 2-phenoxy-6,7,8,9-tetrahydro-5H- pyrazino[2,3-d]azepine |
| 22 | 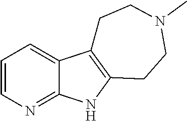 | 7-methyl-5,6,7,8,9,10- hexahydropyrido[3′,2′:4,5]pyrrolo[2,3- d]azepine |
| 23 | 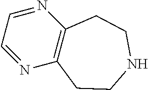 [SMILES: c1cnc2c(n1)CCNCC2] | 6,7,8,9-tetrahydro-5H-pyrazino[2,3- d]azepine |
| 24 | 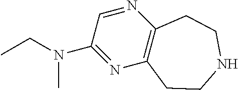 | N-ethyl-N-methyl-6,7,8,9-tetrahydro- 5H-pyrazino[2,3-d]azepin-2-amine |
| 25 | 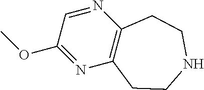 | 2-methoxy-6,7,8,9-tetrahydro-5H- pyrazino[2,3-d]azepine |
| 26 | 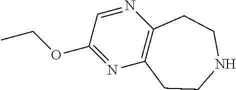 | 2-ethoxy-6,7,8,9-tetrahydro-5H- pyrazino[2,3-d]azepine |
| 27 | 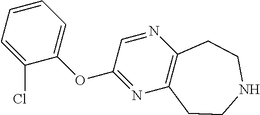 [SMILES: Clc1ccccc1Oc1cnc2c(n1)CCNCC2] | 2-(2-chlorophenoxy)-6,7,8,9-tetrahydro- 5H-pyrazino[2,3-d]azepine |
| 28 | 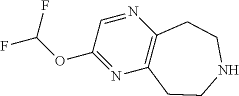 [SMILES: FC(F)Oc1cnc2c(n1)CCNCC2] | 2-(difluoromethxoy)-6,7,8,9-tetrahydro- 5H-pyrazino[2,3-d]azepine |
| 29 | 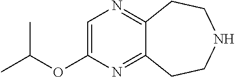 | 2-isopropoxy-6,7,8,9-tetrahydro-5H- pyrazino[2,3-d]azepine |
| 30 | 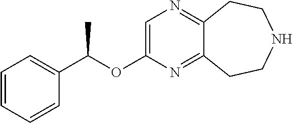 [SMILES: C[C@@H](Oc1cnc2c(n1)CCNCC2)c1ccccc1] | (R)-2-(1-phenylethoxy)-6,7,8,9- tetrahydro-5H-pyrazino[2,3-d]azepine |
| 31 | 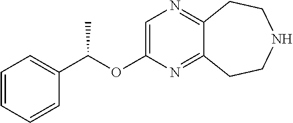 [SMILES: C[C@H](Oc1cnc2c(n1)CCNCC2)c1ccccc1] | (S)-2-(1-phenylethoxy)-6,7,8,9- tetrahydro-5H-pyrazino[2,3-d]azepine |

In an aspect, the present disclosure provides a compound, or pharmaceutically acceptable salt or solvate thereof, having the structure of Formula (I):

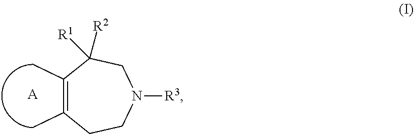

wherein,

R¹, R² and R³ are each independently selected from H and C₁-C₆ optionally substituted alkyl; and ring A is an optionally substituted heteroaryl ring selected from

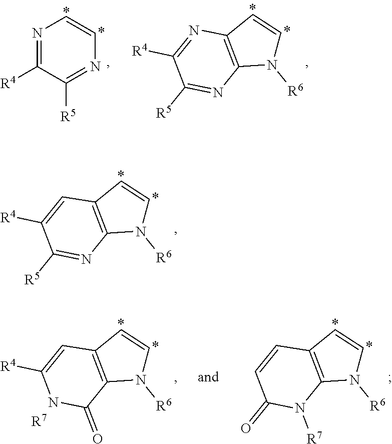

wherein
* represents the points of attachment;

R⁴ and R⁵ are independently selected from H, halo, CN, C₁₋₄alkyl, C₁₋₄haloalkyl, OC₁₋₄alkyl, OC₁₋₄haloalkyl, and N(R⁸R⁹);

R⁶ and R⁷ are independently selected from H and C₁-C₆ optionally substituted alkyl; and

R⁸ and R⁹ are joined to form, together with the atom therebetween, a C₃₋₁₂heterocycloalkyl, optionally comprising one additional heteromoiety selected from N(R¹⁰), O, S, S(O) and SO₂, and optionally substituted with one or more substituents selected from halo, ═O, OH, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), N(C₁₋₆alkyl)(C₁₋₆alkyl), SC₁₋₆alkyl, S(O)C₁₋₆alkyl, and SO₂C₁₋₆alkyl, wherein

R¹⁰ is selected from hydrogen, C₁₋₆alkyl, C₃₋₆cycloalkyl, aryl, C₃₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, C(O)NH₂, C(O)NH(C₁₋₆alkyl), C(O)N(C₁₋₆alkyl)(C₁₋₆alkyl), S(O)C₁₋₆alkyl, and SO₂C₁₋₆alkyl; and

all alkyl, alkylene, cycloalkyl, heterocycloalkyl, aryl and heteroaryl groups of the optional substituents on the C₃₋₁₂heterocycloalkyl formed by R⁸ and R⁹ are optionally substituted with one or more substituents selected from halo, C₁₋₆alkyl, OC₁₋₆alkyl, C₁₋₆haloalkyl, and OC₁₋₆haloalkyl.

In some embodiments, R¹ is H. In some embodiments, R¹ is H, and R² is C₁₋₆ alkyl. In some embodiments, R¹ is H, and R² is methyl. In some embodiments, ring A

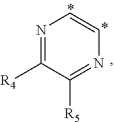

R¹ is H, and R² is C₁₋₆ alkyl. In some embodiments, ring A is

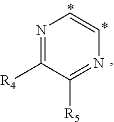

R¹ is H, and R² is methyl.

In some embodiments, R³ is selected from H and C₁₋₆ alkyl. In some embodiments, ring A is

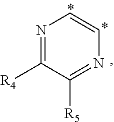

and R³ is H. In some embodiments, ring A

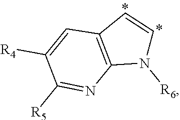

and R³ is selected from H and C₁₋₆ alkyl. In some embodiments, ring A is

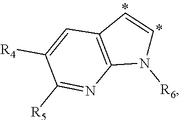

and R³ is selected from H and methyl. In some embodiments, ring A is

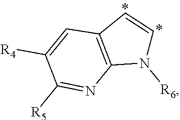

and R³ is methyl.

In some embodiments, ring A is an optionally substituted 3- to 10-membered heterocycle, C₃₋₁₀ carbocycle, 3- to 10-membered heteroaryl, or C₃₋₁₀ aryl. In some embodiments, ring A is an optionally substituted 3- to 10-membered heterocycle. In some embodiments, ring A is an optionally substituted 3- to 10-membered heteroarene. In some embodiments, ring A is selected from

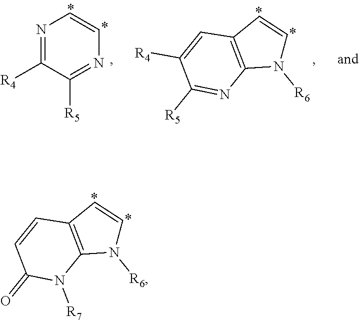

wherein * represents the points of attachment. In some embodiments, ring A is selected from

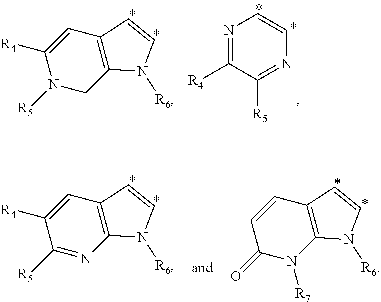

In some embodiments, ring A is

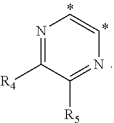

In some embodiments, ring A is

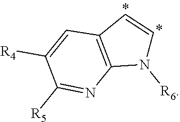

In some embodiments, ring A is

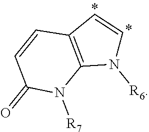

In some embodiments, ring A is

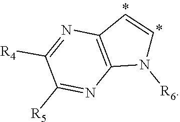

In some embodiments, ring A is

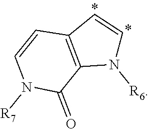

In some embodiments, ring A is

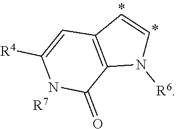

In some embodiments, R⁴ and R⁵ are independently selected from H, halo, CN, C₁₋₄alkyl, C₁₋₄haloalkyl, OC₁₋₄alkyl, OC₁₋₄haloalkyl, and N(R⁸R⁹). In some embodiments, R⁴ and R⁵ are independently selected from H, halo, C₁₋₄alkyl, C₁₋₄haloalkyl, OC₁₋₄alkyl, OC₁₋₄haloalkyl, and N(R⁸R⁹). In some embodiments, R⁴ and R⁵ are independently selected from H, halo, C₁ alkyl, C₁₋₄haloalkyl, OC₁₋₄alkyl, OC₁₋₄haloalkyl, and N(R⁸R⁹). In some embodiments, R⁴ and R⁵ are independently selected from H, halo, C₁₋₄haloalkyl, OC₁₋₄alkyl, OC₁₋₄haloalkyl, and N(R⁸R⁹).

In some embodiments, R⁴ and R⁵ are independently selected from H and N(R⁸R⁹). In some embodiments, exactly one of R⁴ and R⁵ is H, and exactly one of R⁴ and R⁵ is N(R⁸R⁹). In some embodiments, exactly one of R⁴ and R⁵ is H, and exactly one of R⁴ and R⁵ is N(R⁸R⁹). In some embodiments, R⁴ is H, and R⁵ is N(R⁸R⁹).

In some embodiments, ring A is

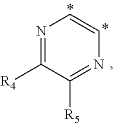

R⁴ is H, and R⁵ is N(R⁸R⁹). In some embodiments, ring A is

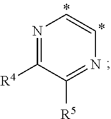

and R⁴ and R⁵ are each independently selected from H, NO₂, F, CN, C₁₋₄alkyl, C₁₋₄haloalkyl, OC₁₋₄alkyl, OC₁₋₄haloalkyl, and N(R⁸R⁹).

In some embodiments, ring A is

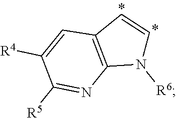

and R⁴ and R⁵ are each independently selected from H, NO₂, F, CN, C₂₋₄alkyl, C₁₋₄haloalkyl, OC₂₋₄alkyl, O(optionally substituted C₃₋₁₀ carbocycle), O(optionally substituted C₃₋₁₀ aryl), O(3- to 10-membered heterocycle), O(3- to 10-membered heteroaryl), OC₁₋₄haloalkyl, and N(R⁸R⁹).

In some embodiments, R⁸ and R⁹ are joined to form, together with the atom therebetween, a C₃₋₁₂ heterocycloalkyl. In some embodiments, the C₃₋₁₂ heterocycloalkyl formed by R⁸ and R⁹ contains additional heteromoieties. In some embodiments, the C₃₋₁₂ heterocycloalkyl formed by R⁸ and R⁹ contains multiple additional heteromoieties selected from N(R¹⁰), O, S, S(O) and SO₂. In some embodiments, the C₃₋₁₂ heterocycloalkyl formed by R⁸ and R⁹ contains one, two, three, four, or five additional heteromoieties selected from N(R¹⁰), O, S, S(O) and SO₂. In some embodiments, the C₃₋₁₂ heterocycloalkyl formed by R⁸ and R⁹ contains one additional heteromoiety selected from N(R¹⁰), O, S, S(O) and SO₂. In some embodiments, the C₃₋₁₂ heterocycloalkyl formed by R⁸ and R⁹ contains one additional heteromoiety selected from N(R¹⁰), O, and S. In some embodiments, the C₃₋₁₂ heterocycloalkyl formed by R⁸ and R⁹ contains one additional heteromoiety selected from N(R¹⁰), and O. In some embodiments, R, and R⁹ are joined to form, together with the atom therebetween, a C₃₋₁₂heterocycloalkyl. In some embodiments, the C₃₋₁₂ heterocycloalkyl formed by R⁸ and R⁹ contains one additional oxygen atom.

In some embodiments, the C₃₋₁₂ heterocycloalkyl formed by R⁸ and R⁹ is optionally substituted with one or more substituents selected from halo, ═O, OH, C₁₋₆alkyl, C₃₋₆cycloalkyl, aryl, C₅-6heteroaryl, C₃₋₆ heterocycloalkyl, C₁₋₆ alkyleneC₃₋₆cycloalkyl, C₁₋₆alkylenearyl, C₁₋₆alkyleneC₅-6heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), and N(C₁₋₆alkyl)(C₁₋₆alkyl). In some embodiments, the C₃₋₁₂heterocycloalkyl formed by R⁸ and R⁹ is optionally substituted with one or more substituents selected from halo, ═O, OH, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, OC₁₋₆alkyl, NH₂, NH(C₁₋₆alkyl), and N(C₁₋₆alkyl)(C₁₋₆alkyl). In some embodiments, the C₃₋₁₂heterocycloalkyl formed by R⁸ and R⁹ is optionally substituted with one or more substituents selected from halo, ═O, OH, OC₁₋₆alkyl, NH₂, and NH(C₁₋₆alkyl). In some embodiments, the C₃₋₁₂heterocycloalkyl formed by R⁸ and R⁹ is optionally substituted with one or more substituents selected from halo, ═O, OH, OC₁alkyl, NH₂, and NH(C₁alkyl). In some embodiments, the C₃₋₁₂heterocycloalkyl formed by R⁸ and R⁹ is optionally substituted with one or more substituents selected from halo, ═O, OH, OC₁alkyl, NH₂, and NH(C₁alkyl). In some embodiments, the C₃₋₁₂heterocycloalkyl formed by R⁸ and R⁹ is optionally substituted with one or more substituents selected from fluoro, ═O, OH, OC₁alkyl, NH₂, and NH(C₁alkyl). In some embodiments, the C₃₋₁₂heterocycloalkyl formed by R⁸ and R⁹ is optionally substituted with one or more fluoro substituents.

In some embodiments, ring A is

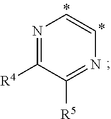

and the one additional heteromoiety of the C₃₋₁₂ heterocycloalkyl formed by R⁸ and R⁹ is NR¹⁰. In some embodiments, ring A is R⁵ the one additional heteromoiety of the C₃₋₁₂heterocycloalkyl formed by R⁸ and R⁹ is NR¹⁰; and the C₃₋₁₂heterocycloalkyl formed by R⁸ and R⁹ is optionally substituted with one or more substituents selected from halo, OH, NO₂, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), N(C₁₋₆alkyl)(C₁₋₆alkyl), SC₁₋₆alkyl, S(O)C₁₋₆alkyl, and SO₂C₁₋₆alkyl. In some embodiments, ring A is

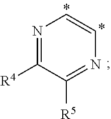

the one additional heteromoiety of the C₃₋₁₂heterocycloalkyl formed by R⁸ and R⁹ is NR¹⁰; and the C₃₋₁₂heterocycloalkyl formed by R⁸ and R⁹ is optionally substituted with one or more substituents selected from halo, OH, NO₂, C₁₋₆alkyl, C₃₋₆cycloalkyl, aryl, C₅₋₆heteroaryl, C₃₋₆ heterocycloalkyl, and OC₁₋₆alkyl. In some embodiments, ring A is

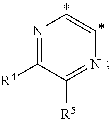

the one additional heteromoiety of the C₃₋₁₂heterocycloalkyl formed by R⁸ and R⁹ is NR¹⁰; and R¹⁰ is selected from C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, C(O)NH₂, C(O)NH(C₁₋₆alkyl), C(O)N(C₁₋₆alkyl)(C₁₋₆alkyl), S(O)C₁₋₆alkyl, and SO₂C₁₋₆alkyl. In some embodiments, ring A is

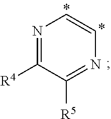

some embodiments, ring A is

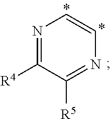

the one additional heteromoiety of the C₃₋₁₂heterocycloalkyl formed by R⁸ and R⁹ is NR¹⁰; and R¹⁰ is selected from C₁₋₆alkyl, C₃₋₆cycloalkyl, aryl, C₅₋₆heteroaryl, and C₃₋₆ heterocycloalkyl. In some embodiments, ring A is

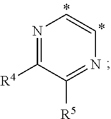

the one additional heteromoiety of the C₃₋₁₂heterocycloalkyl formed by R⁸ and R⁹ is NR¹⁰; and R¹⁰ is selected from C₁₋₆alkyl.

In some embodiments, ring A is

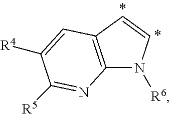

and the C₃₋₁₂heterocycloalkyl formed by R⁸ and R⁹ is optionally substituted with one or more substituents selected from halo, NO₂, OH, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), N(C₁₋₆alkyl)(C₁₋₆alkyl), SC₁₋₆alkyl, S(O)C₁₋₆alkyl, and SO₂C₁₋₆alkyl. In some embodiments, ring A is

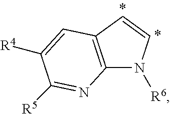

and the C₃₋₁₂heterocycloalkyl formed by R⁸ and R⁹ is optionally substituted with one or more substituents selected from halo, NO₂, OH, C₁₋₆alkyl, C₃₋₆cycloalkyl, aryl, C₅-6heteroaryl, C₃₋₆ heterocycloalkyl, C₁₋₆ alkyleneC₃₋₆cycloalkyl, C₁₋₆alkylenearyl, C₁₋₆alkyleneC₅-6heteroaryl, C₁₋₆alkyleneC₃₋₆heterocycloalkyl, C(O)C₁₋₆alkyl, OC₁₋₆alkyl, NH₂, and NH(C₁₋₆ alkyl). In some embodiments, ring A is

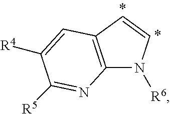

and the C₃₋₁₂heterocycloalkyl formed by R⁸ and R⁹ is optionally substituted with one or more substituents selected from halo, NO₂, OH, C₁₋₆alkyl, C₃₋₆cycloalkyl, aryl, C₅₋₆heteroaryl, C₃₋₆ heterocycloalkyl, C(O)C₁₋₆alkyl, OC₁₋₆ alkyl, NH₂, and NH(C₁₋₆alkyl). In some embodiments, ring A is

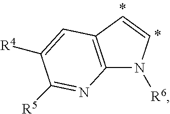

and the C₃₋₁₂heterocycloalkyl formed by R⁸ and R⁹ is optionally substituted with one or more substituents selected from halo, NO₂, OH, C₁₋₆alkyl, OC₁₋₆alkyl, NH₂, and NH(C₁₋₆alkyl). In some embodiments, R¹⁰ is selected from hydrogen, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, and C₁₋₆alkyleneC₃₋₆heterocycloalkyl.

In some embodiments, R¹⁰ is selected from hydrogen, C₁₋₆alkyl, C₃₋₆cycloalkyl, aryl, C₅-6heteroaryl, and C₃₋₆ heterocycloalkyl. In some embodiments, R¹⁰ is selected from hydrogen, and C₁₋₆alkyl.

In some embodiments, the heterocycle formed by R⁸ and R⁹ is selected from

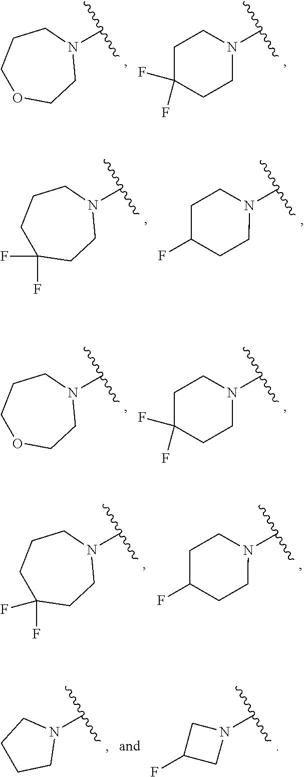

In some embodiments, R⁸ and R⁹ are methyl. In some embodiments, R⁸ is methyl, and R⁹ is ethyl.

In some embodiments, R¹ and R² are H; R³ is selected from H and C₁-C₆ optionally substituted alkyl; ring A

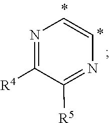

R⁴ and R⁵ are each independently selected from H, C₁₋₄haloalkyl, OC₁₋₄alkyl, OC₁₋₄haloalkyl, O(optionally substituted C₃₋₁₀ carbocycle), O(optionally substituted C₃₋₁₀ aryl), and N(R⁸R⁹); R⁸ and R⁹ are joined to form, together with the atom therebetween, a C₃₋₁₂heterocycloalkyl. In some embodiments, the C₃₋₁₂heterocycloalkyl comprises one additional heteromoiety selected from N(R¹⁰), O, S, S(O) and SO₂, and optionally substituted with one or more substituents selected from halo, OH, NO₂, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), N(C₁₋₆alkyl)(C₁₋₆alkyl), SC₁₋₆alkyl, S(O)C₁₋₆alkyl, and SO₂C₁₋₆alkyl, wherein R¹⁰ is selected from C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆ alkyleneC₃₋₆heterocycloalkyl, C(O)C₁₋₆alkyl, C(O)NH₂, C(O)NH(C₁₋₆alkyl), C(O)N(C₁₋₆alkyl)(C₁₋₆alkyl), S(O)C₁₋₆alkyl, and SO₂C₁₋₆alkyl.

In some embodiments, R¹ and R² are H; R³ is selected from H and C₁-C₆ optionally substituted alkyl; ring A is

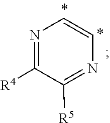

R⁴ and R⁵ are each independently selected from H, C₁₋₄haloalkyl, OC₁₋₄alkyl, OC₁₋₄haloalkyl, O(optionally substituted C₃₋₁₀ carbocycle), O(optionally substituted C₃₋₁₀ aryl), and N(R⁸R⁹); R⁸ and R⁹ are joined to form, together with the atom therebetween, a C₃₋₁₂heterocycloalkyl, optionally comprising one additional heteromoiety selected from N(R¹⁰) and O and optionally substituted with one or more substituents selected from halo, OH, NO₂, C₁₋₆alkyl, C₃₋₆cycloalkyl, aryl, C₅₋₆heteroaryl, C₃₋₆ heterocycloalkyl, C(O)C₁₋₆alkyl, OC₁₋₆alkyl, NH₂, and NH(C₁₋₆alkyl), wherein R¹⁰ is selected from C₁₋₆alkyl, C₃₋₆cycloalkyl, aryl, C₅-6heteroaryl, C₃₋₆ heterocycloalkyl, C(O)C₁₋₆alkyl, C(O)NH₂, and C(O)NH(C₁₋₆alkyl).

In some embodiments, R¹ and R² are H; R³ is selected from H and C₁-C₆ optionally substituted alkyl; ring A is

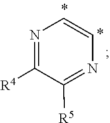

R⁴ and R⁵ are each independently selected from H, C₁₋₄haloalkyl, OC₁₋₄alkyl, and N(R⁸R⁹); R⁸ and R⁹ are joined to form, together with the atom therebetween, a C₃₋₁₂heterocycloalkyl, optionally comprising one additional oxygen atom and optionally substituted with one or more substituents selected from halo, OH, NO₂, C₁₋₆alkyl, aryl, C₅-6heteroaryl, C₃₋₆ heterocycloalkyl, OC₁₋₆alkyl, NH₂, and NH(C₁₋₆alkyl).

In some embodiments, R¹ and R² are H; R³ is selected from H and C₁-C₆ optionally substituted alkyl; ring A is

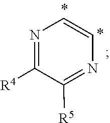

R⁴ and R⁵ are each independently selected from H, C₁₋₄haloalkyl, OC₁₋₄alkyl, and N(R⁸R⁹); R⁸ and R⁹ are joined to form, together with the atom therebetween, a C₃₋₁₂heterocycloalkyl, optionally comprising one additional oxygen atom and optionally substituted with one or more substituents selected from halo, OH, NO₂, C₁₋₆alkyl, aryl, and C₅-6heteroaryl.

In some embodiments, R¹ and R² are H; R³ is selected from H and C₁-C₆ optionally substituted alkyl; ring A is

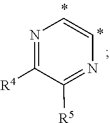

R⁴ and R⁵ are each independently selected from H and N(R⁸R⁹); R⁸ and R⁹ are joined to form, together with the atom therebetween, a C₃₋₁₂heterocycloalkyl, optionally comprising one additional oxygen atom and optionally substituted with one or more substituents selected from halo, OH, NO₂, C₁₋₆alkyl, aryl, and C₅₋₆heteroaryl.

In some embodiments, R¹ and R² are H; R³ is selected from H and C₁-C₆ optionally substituted alkyl; ring A is

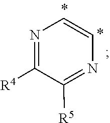

R⁴ and R⁵ are each independently selected from H and N(R⁸R⁹); R⁸ and R⁹ are joined to form, together with the atom therebetween, a C₃₋₁₂heterocycloalkyl, optionally comprising one additional oxygen atom and optionally substituted with one or more substituents selected from halo, OH, and C₁₋₆alkyl.

In some embodiments, R¹ and R² are H; R³ is selected from H and C₁-C₆ optionally substituted alkyl; ring A

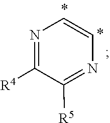

R⁴ and R⁵ are each independently selected from H and N(R⁸R⁹); R⁸ and R⁹ are joined to form, together with the atom therebetween, a C₃₋₁₂heterocycloalkyl, optionally comprising one additional oxygen atom and optionally substituted with one or more substituents selected from halo, and OH.

In some embodiments, R¹ and R² are H; R³ is selected from H and C₁-C₆ optionally substituted alkyl; ring A is

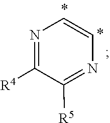

R⁴ and R⁵ are each independently selected from H and N(R⁸R⁹); R⁸ and R⁹ are joined to form, together with the atom therebetween, a C₃₋₁₂heterocycloalkyl, optionally comprising one additional oxygen atom and optionally substituted with one or more halo substituents.

In some embodiments, R¹ and R² are H; R³ is selected from H and C₁-C₆ optionally substituted alkyl; ring A is

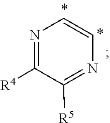

R⁴ and R⁵ are each independently selected from H and N(R⁸R⁹); R⁸ and R⁹ are joined to form, together with the atom therebetween, a C₃₋₁₂heterocycloalkyl, optionally comprising one additional oxygen atom and optionally substituted with one or more fluoro substituents.

In some embodiments, R¹ and R² are H; R³ is selected from H and C₁-C₆ optionally substituted alkyl; ring A is

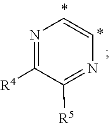

R⁴ and R⁵ are each independently selected from H, C₁₋₄haloalkyl, OC₁₋₄alkyl, OC₁₋₄haloalkyl, O(optionally substituted C₃₋₁₀ carbocycle), O(optionally substituted C₃₋₁₀ aryl), and N(R⁸R⁹); R⁸ and R⁹ are joined to form, together with the atom therebetween, a C₃₋₁₂heterocycloalkyl, optionally comprising one additional oxygen atom and optionally substituted with one or more substituents selected from halo, OH, NO₂, C₁₋₆alkyl, C₃₋₆cycloalkyl, aryl, C₅₋₆heteroaryl, C₃₋₆ heterocycloalkyl, C(O)C₁₋₆alkyl, OC₁₋₆alkyl, OC₁₋₆alkyleneOC₁₋₆alkyl, NH₂, and NH(C₁₋₆alkyl).

In an aspect, the present disclosure provides a compound, or pharmaceutically acceptable salt or solvate thereof, having the structure of Formula (I):

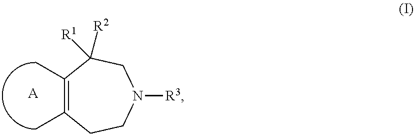

wherein

R¹, R² and R³ are each independently selected from H and C₁-C₆ optionally substituted alkyl; and

ring A is an optionally substituted heteroaryl ring selected from

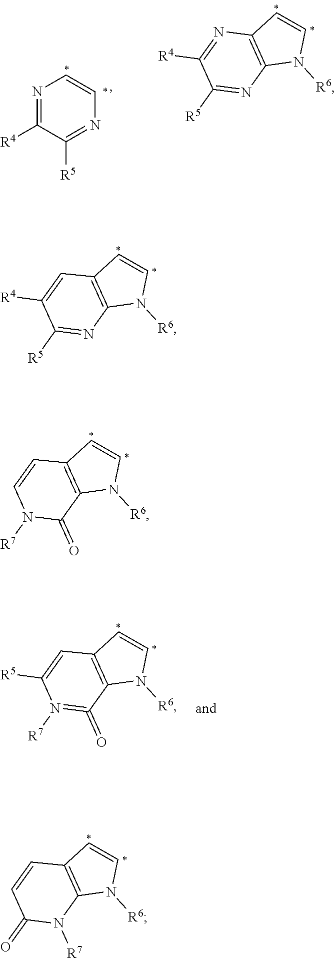

wherein
* represents the points of attachment;

R⁴ and R⁵ are each independently selected from H, NO₂, halo, CN, C₁₋₄ alkyl, C₁₋₄haloalkyl, OC₁₋₄alkyl, O(optionally substituted C₃₋₁₀ carbocycle), O(optionally substituted C₃₋₁₀ aryl), O(3 to 10-membered heterocycle), O(3- to 10-membered heteroaryl), OC₁₋₄haloalkyl, and N(R⁸R⁹), wherein

each OC₁₋₄alkyl, OC₃₋₁₀aryl, and O(3- to 10-membered heteroaryl), of R⁴ and R⁵ is independently optionally substituted with one or more substituents selected from C₃₋₆ carbocycle, C₃₋₆ aryl, 3- to 10-membered heterocycle, and 3- to 10-membered heteroaryl, wherein each C₃₋₆ carbocycle, C₃₋₆ aryl, 3- to 10-membered heterocycle, and 3- to 10-membered heteroaryl of R⁴ and R⁵, is optionally substituted with one or more substituents selected from halogen, C₁₋₆ alkyl, C₁₋₆aminoalkyl, CN, NO₂, OH, and C₁₋₆ alkoxy;

when ring A is

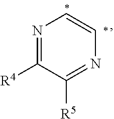

R⁴ and R⁵ are each independently selected from H, NO₂, F, CN, C₁₋₄alkyl, C₁₋₄haloalkyl, OC₁₋₄alkyl, OC₁₋₄haloalkyl, O(optionally substituted C₃₋₁₀ carbocycle), O(optionally substituted C₃₋₁₀ aryl), O(3- to 10-membered heterocycle), O(3- to 10-membered heteroaryl), and N(R⁸R⁹); and

when ring A is

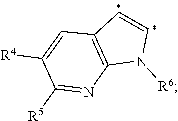

R⁴ and R⁵ are each independently selected from H, NO₂, F, CN, C₂₋₄alkyl, C₁₋₄haloalkyl, OC₂₋₄alkyl, O(optionally substituted C₃₋₁₀ carbocycle), O(optionally substituted C₃₋₁₀ aryl), O(3- to 10-membered heterocycle), O(3- to 10-membered heteroaryl), OC₁₋₄haloalkyl, and N(R⁸R⁹);

R⁶ and R⁷ are each independently selected from H and C₁-C₆ optionally substituted alkyl; and

R⁸ and R⁹ are each independently selected from hydrogen, NO₂, C(O)(C₁₋₆ optionally substituted alkyl), C(O)N(C₁₋₆ optionally substituted alkyl)₂, C(O)O(C₁₋₆ optionally substituted alkyl), S(O)(C₁₋₆ optionally substituted alkyl), and S(O)₂(C₁₋₆ optionally substituted alkyl), wherein
when ring A is

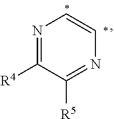

and R⁸ is H; R⁹ is selected from
C₁₋₆ alkyl, C(O)N(C₁₋₆ optionally substituted alkyl)₂, C(O)O(C₁₋₆ optionally substituted alkyl), S(O)(C₁₋₆ optionally substituted alkyl), and S(O)₂(C₁₋₆ optionally substituted alkyl); and C₃₋₁₀ carbocycle, C₃₋₁₀ aryl, 3- to 10-membered heteroaryl, and 3- to 10-membered heterocycle, optionally substituted with one or more substituents selected from halo, C₁₋₆alkyl, OC₁₋₆alkyl, C₁₋₆haloalkyl, and OC₁₋₆haloalkyl; and C₁₋₆ alkyl, optionally substituted with one or more substituents selected from halo, C₁₋₆alkyl, OC₁₋₆alkyl, C₁₋₆haloalkyl, and OC₁₋₆haloalkyl; and C₃₋₁₀ carbocycle, C₃₋₁₀ aryl, 3- to 10-membered heteroaryl, and 3- to 10-membered heterocycle, optionally substituted with one or more substituents selected from halo, C₁₋₆alkyl, OC₁₋₆alkyl, C₁₋₆haloalkyl, and OC₁₋₆haloalkyl; or

R⁸ and R⁹ are joined to form, together with the atom therebetween, a C₃₋₁₂heterocycloalkyl, optionally substituted with one or more substituents selected from NO₂, CN, halo, ═O, OH, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), N(C₁₋₆alkyl)(C₁₋₆alkyl), SC₁₋₆alkyl, S(O)C₁₋₆alkyl, and SO₂C₁₋₆alkyl, wherein

R¹⁰ is selected from hydrogen, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, C(O)NH₂, C(O)NH(C₁₋₆alkyl), C(O)N(C₁₋₆alkyl)(C₁₋₆alkyl), S(O)C₁₋₆alkyl, and SO₂C₁₋₆alkyl;

all alkyl, alkylene, cycloalkyl, heterocycloalkyl, aryl and heteroaryl groups of the optional substituents on the C₃₋₁₂heterocycloalkyl formed by R⁸ and R⁹ are optionally substituted with one or more substituents selected from halo, C₁₋₆alkyl, OC₁₋₆alkyl, C₁₋₆haloalkyl, and OC₁₋₆haloalkyl;

when ring A is

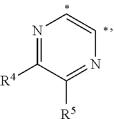

and the one additional heteromoiety of the C₃₋₁₂heterocycloalkyl formed by R⁸ and R⁹ is NR¹⁰;

the C₃₋₁₂heterocycloalkyl formed by R⁸ and R⁹ is optionally substituted with one or more substituents selected from halo, OH, NO₂, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), N(C₁₋₆alkyl)(C₁₋₆alkyl), SC₁₋₆alkyl, S(O)C₁₋₆alkyl, and SO₂C₁₋₆alkyl; and R¹⁰ is selected from C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, C(O)NH₂, C(O)NH(C₁₋₆alkyl), C(O)N(C₁₋₆alkyl)(C₁₋₆alkyl), S(O)C₁₋₆alkyl, and SO₂C₁₋₆alkyl; and
when ring A is

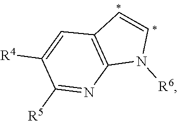

the C₃₋₁₂heterocycloalkyl formed by R⁸ and R⁹ is optionally substituted with one or more substituents selected from halo, NO₂, OH, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), N(C₁₋₆alkyl)(C₁₋₆alkyl), SC₁₋₆alkyl, S(O)C₁₋₆alkyl, and SO₂C₁₋₆alkyl.

In some embodiments, R¹ is H. In some embodiments, R¹ is H, and R² is C₁₋₆ alkyl. In some embodiments, R¹ is H, and R² is methyl. In some embodiments, ring A is

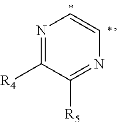

R¹ is H, and R² is C₁₋₆ alkyl. In some embodiments, ring A is

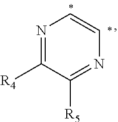

R¹ is H, and R² is methyl.

In some embodiments, R³ is selected from H and C₁₋₆ alkyl. In some embodiments, ring A is

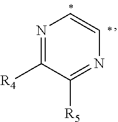

and R³ is H. In some embodiments, ring A is

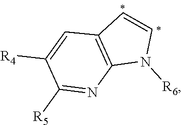

and R³ is selected from H and C₁₋₆ alkyl. In some embodiments, ring A is

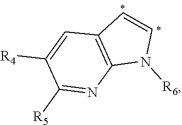

and R³ is selected from H and methyl. In some embodiments, ring A is

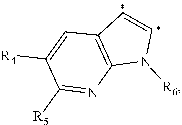

and R³ is methyl.

In some embodiments, ring A is an optionally substituted 3- to 10-membered heterocycle, C₃₋₁₀ carbocycle, 3- to 10-membered heteroaryl, or C₃₋₁₀ aryl. In some embodiments, ring A is an optionally substituted 3- to 10-membered heterocycle. In some embodiments, ring A is an optionally substituted 3- to 10-membered heteroarene. In some embodiments, ring A is selected from

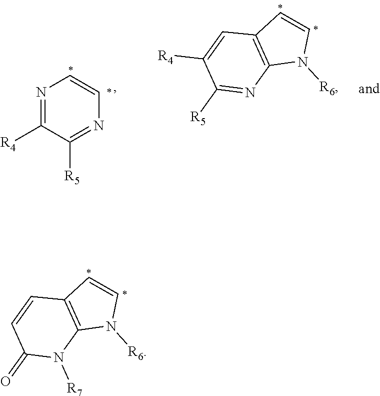

In some embodiments, ring A is selected from

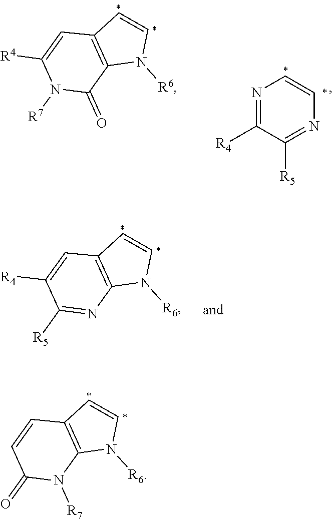

In some embodiments, ring A is

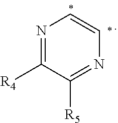

In some embodiments, ring A is

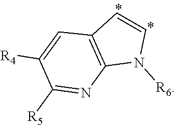

In some embodiments, ring A is

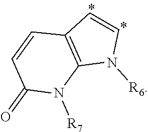

In some embodiments, ring A is

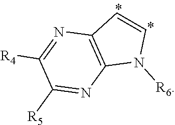

In some embodiments, ring A is

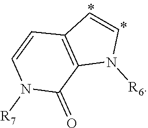

In some embodiments, ring A is

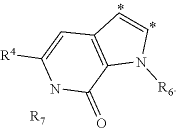

In some embodiments, R⁴ and R⁵ are independently selected from H, halo, CN, C₁₋₄ alkyl, C₁₋₄haloalkyl, OC₁₋₄alkyl, O(optionally substituted C₃₋₁₀ carbocycle), O(optionally substituted C₃₋₁₀ aryl), O(3- to 10-membered heterocycle), O(3- to 10-membered heteroaryl), OC₁₋₄haloalkyl, and N(R⁸R⁹). In some embodiments, R⁴ and R⁵ are independently selected from H, halo, CN, C₁₋₄ alkyl, C₁₋₄haloalkyl, OC₁₋₄alkyl, O(optionally substituted C₃₋₁₀ carbocycle), O(optionally substituted C₃₋₁₀ aryl), OC₁₋₄haloalkyl, and N(R⁸R⁹). In some embodiments, R⁴ and R⁵ are independently selected from H, halo, C₁₋₄haloalkyl, OC₁₋₄alkyl, O(optionally substituted C₃₋₁₀ carbocycle), O(optionally substituted C₃₋₁₀ aryl), OC₁₋₄haloalkyl, and N(R⁸R⁹). In some embodiments, R⁴ and R⁵ are independently selected from H and N(R⁸R⁹). In some embodiments, exactly one of R⁴ and R⁵ is H, and exactly one of R⁴ and R⁵ is N(R⁸R⁹). In some embodiments, exactly one of R⁴ and R⁵ is H, and exactly one of R⁴ and R⁵ is N(R⁸R⁹). In some embodiments, R⁴ is H, and R⁵ is N(R⁸R⁹). In some embodiments, ring A is

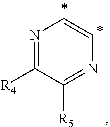

R⁴ is H, and R⁵ is N(R⁸R⁹).

In some embodiments, ring A is

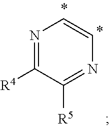

and R⁴ and R⁵ are each independently selected from H, NO₂, halo, CN, C₁₋₄ alkyl, C₁₋₄haloalkyl, OC₁₋₄alkyl, O(optionally substituted C₃₋₁₀ carbocycle), O(optionally substituted C₃₋₁₀ aryl), O(3- to 10-membered heterocycle), O(3- to 10-membered heteroaryl), OC₁₋₄haloalkyl, and N(R⁸R⁹). In some embodiments, ring A is

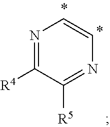

and R⁴ and R⁵ are each independently selected from H, NO₂, halo, CN, C₁₋₄ alkyl, C₁₋₄haloalkyl, OC₁₋₄alkyl, O(optionally substituted C₃₋₁₀ carbocycle), O(optionally substituted C₃₋₁₀ aryl), and OC₁₋₄haloalkyl, and N(R⁸R⁹). In some embodiments, ring A is

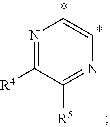

and R⁴ and R⁵ are each independently selected from H, halo, C₁₋₄ alkyl, C₁₋₄haloalkyl, OC₁₋₄alkyl, O(optionally substituted C₃₋₁₀ carbocycle), O(optionally substituted C₃₋₁₀ aryl), and OC₁₋₄ haloalkyl, and N(R⁸R⁹). In some embodiments, ring A is

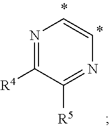

and R⁴ and R⁵ are each independently selected from H, halo, C₁₋₄haloalkyl, OC₁₋₄alkyl, O(optionally substituted C₃₋₁₀ carbocycle), O(optionally substituted C₃₋₁₀ aryl), and OC₁₋₄haloalkyl, and N(R⁸R⁹).

In some embodiments, R⁸ and R⁹ are joined to form, together with the atom therebetween, a C₃₋₁₂heterocycloalkyl. In some embodiments, the C₃₋₁₂ heterocycloalkyl formed by R⁸ and R⁹ contains additional heteromoieties. In some embodiments, the C₃₋₁₂ heterocycloalkyl formed by R⁸ and R⁹ contains multiple additional heteromoieties selected from N(R¹⁰), O, S, S(O) and SO₂.

In some embodiments, the C₃₋₁₂ heterocycloalkyl formed by R⁸ and R⁹ contains one, two, three, four, or five additional heteromoieties selected from N(R¹⁰), O, S, S(O) and SO₂. In some embodiments, the C₃₋₁₂ heterocycloalkyl formed by R⁸ and R⁹ contains one additional heteromoiety selected from N(R¹⁰), O, S, S(O) and SO₂. In some embodiments, the C₃₋₁₂ heterocycloalkyl formed by R⁸ and R⁹ contains one additional heteromoiety selected from N(R¹⁰), O, and S. In some embodiments, the C₃₋₁₂ heterocycloalkyl formed by R⁸ and R⁹ contains one additional heteromoiety selected from N(R¹⁰), and O. In some embodiments, R, and R⁹ are joined to form, together with the atom therebetween, a C₃₋₁₂heterocycloalkyl. In some embodiments, the C₃₋₁₂ heterocycloalkyl formed by R⁸ and R⁹ contains one additional oxygen atom.

In some embodiments, the C₃₋₁₂heterocycloalkyl formed by R⁸ and R⁹ is optionally substituted with one or more substituents selected from NO₂, CN, halo, ═O, OH, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), and N(C₁₋₆alkyl)(C₁₋₆alkyl). In some embodiments, the C₃₋₁₂heterocycloalkyl formed by R⁸ and R⁹ is optionally substituted with one or more substituents selected from NO₂, CN, halo, ═O, OH, C₁₋₆alkyl, C₃₋₆cycloalkyl, aryl, C₅-6heteroaryl, C₃₋₆ heterocycloalkyl, C₁₋₆ alkyleneC₃₋₆cycloalkyl, C₁₋₆alkylenearyl, C₁₋₆alkyleneC₅-6heteroaryl, C₁₋₆alkyleneC₃₋₆heterocycloalkyl, OC₁₋₆alkyl, NH₂, NH(C₁₋₆alkyl), and N(C₁₋₆alkyl)(C₁₋₆alkyl). In some embodiments, the C₃₋₁₂heterocycloalkyl formed by R⁸ and R⁹ is optionally substituted with one or more substituents selected from NO₂, CN, halo, ═O, OH, OC₁₋₆alkyl, NH₂, and NH(C₁₋₆alkyl). In some embodiments, the C₃₋₁₂heterocycloalkyl formed by R⁸ and R⁹ is optionally substituted with one or more substituents selected from NO₂, CN, halo, ═O, OH, OC₁alkyl, NH₂, and NH(C₁alkyl). In some embodiments, the C₃₋₁₂heterocycloalkyl formed by R⁸ and R⁹ is optionally substituted with one or more substituents selected from NO₂, CN, halo, ═O, OH, OC₁alkyl, NH₂, and NH(C₁alkyl). In some embodiments, the C₃₋₁₂heterocycloalkyl formed by R⁸ and R⁹ is optionally substituted with one or more substituents selected from NO₂, CN, fluoro, ═O, OH, OC₁alkyl, NH₂, and NH(C₁alkyl). In some embodiments, the C₃₋₁₂heterocycloalkyl formed by R⁸ and R⁹ is optionally substituted with one or more substituents selected from fluoro, ═O, OH, OC₁alkyl, NH₂, and NH(C₁alkyl).

In some embodiments, the C₃₋₁₂heterocycloalkyl formed by R⁸ and R⁹ is optionally substituted with one or more fluoro substituents.

In some embodiments, ring A is

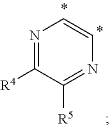

and the one additional heteromoiety of the C₃₋₁₂ heterocycloalkyl formed by R⁸ and R⁹ is NR¹⁰. In some embodiments, ring A is

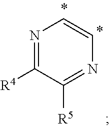

the one additional heteromoiety of the C₃₋₁₂heterocycloalkyl formed by R⁸ and R⁹ is NR¹⁰; and the C₃₋₁₂heterocycloalkyl formed by R⁸ and R⁹ is optionally substituted with one or more substituents selected from halo, OH, NO₂, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), N(C₁₋₆alkyl)(C₁₋₆alkyl), SC₁₋₆alkyl, S(O)C₁₋₆alkyl, and SO₂C₁₋₆alkyl. In some embodiments, ring A is

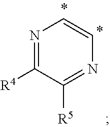

the one additional heteromoiety of the C₃₋₁₂heterocycloalkyl formed by R⁸ and R⁹ is NR¹⁰; and the C₃₋₁₂heterocycloalkyl formed by R⁸ and R⁹ is optionally substituted with one or more substituents selected from halo, OH, NO₂, C₁₋₆alkyl, C₃₋₆cycloalkyl, aryl, C₅₋₆heteroaryl, C₃₋₆ heterocycloalkyl, and OC₁₋₆alkyl. In some embodiments, ring A is

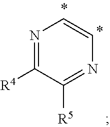

the one additional heteromoiety of the C₃₋₁₂heterocycloalkyl formed by R⁸ and R⁹ is NR¹⁰; and R¹⁰ is selected from C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, C(O)NH₂, C(O)NH(C₁₋₆alkyl), C(O)N(C₁₋₆alkyl)(C₁₋₆alkyl), S(O)C₁₋₆alkyl, and SO₂C₁₋₆alkyl. In some embodiments, ring A is

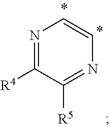

the one additional heteromoiety of the C₃₋₁₂heterocycloalkyl formed by R⁸ and R⁹ is NR¹⁰; and R¹⁰ is selected from C₁₋₆alkyl, C₃₋₆cycloalkyl, aryl, C₅₋₆heteroaryl, and C₃₋₆ heterocycloalkyl. In some embodiments, ring A is

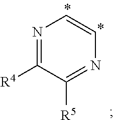

the one additional heteromoiety of the C₃₋₁₂heterocycloalkyl formed by R⁸ and R⁹ is NR¹⁰; and R¹⁰ is selected from C₁₋₆alkyl.

In some embodiments, ring A is

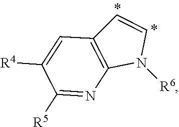

and the C₃₋₁₂heterocycloalkyl formed by R⁸ and R⁹ is optionally substituted with one or more substituents selected from halo, NO₂, OH, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆ alkyl), NHC(O)C₁₋₆alkyl, N(C₁₋₆alkyl)C(O)C₁₋₆alkyl, NH₂, NH(C₁₋₆alkyl), N(C₁₋₆alkyl)(C₁₋₆alkyl), SC₁₋₆alkyl, S(O)C₁₋₆alkyl, and SO₂C₁₋₆alkyl. In some embodiments, ring A is

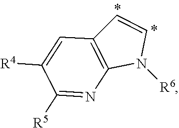

and the C₃₋₁₂heterocycloalkyl formed by R⁸ and R⁹ is optionally substituted with one or more substituents selected from halo, NO₂, OH, C₁₋₆alkyl, C₃₋₆cycloalkyl, aryl, C₅-6heteroaryl, C₃₋₆ heterocycloalkyl, C₁₋₆ alkyleneC₃₋₆cycloalkyl, C₁₋₆alkylenearyl, C₁₋₆alkyleneC₅-6heteroaryl, C₁₋₆alkyleneC₃₋₆heterocycloalkyl, C(O)C₁₋₆alkyl, OC₁₋₆alkyl, NH₂, and NH(C₁₋₆ alkyl). In some embodiments, ring A is

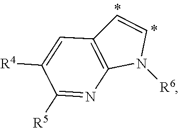

and the C₃₋₁₂heterocycloalkyl formed by R⁸ and R⁹ is optionally substituted with one or more substituents selected from halo, NO₂, OH, C₁₋₆alkyl, C₃₋₆cycloalkyl, aryl, C₅₋₆heteroaryl, C₃₋₆ heterocycloalkyl, C(O)C₁₋₆alkyl, OC₁₋₆ alkyl, NH₂, and NH(C₁₋₆alkyl). In some embodiments, ring A is

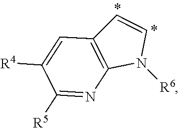

and the C₃₋₁₂heterocycloalkyl formed by R⁸ and R⁹ is optionally substituted with one or more substituents selected from halo, NO₂, OH, C₁₋₆alkyl, OC₁₋₆alkyl, NH₂, and NH(C₁₋₆alkyl).

In some embodiments, R¹⁰ is selected from hydrogen, C₁₋₆alkyl, C₃₋₆cycloalkyl, aryl, C₅-6heteroaryl, C₃₋₆ heterocycloalkyl, C₁₋₆ alkyleneC₃₋₆cycloalkyl, C₁₋₆alkylenearyl, C₁₋₆alkyleneC₅-6heteroaryl, and C₁₋₆alkyleneC₃₋₆heterocycloalkyl. In some embodiments, R¹⁰ is selected from hydrogen, C₁₋₆alkyl, C₃₋₆cycloalkyl, aryl, C₅₋₆heteroaryl, and C₃₋₆ heterocycloalkyl. In some embodiments, R¹⁰ is selected from hydrogen, and C₁₋₆alkyl.

In some embodiments, the heterocycle formed by R⁸ and R⁹ is selected from

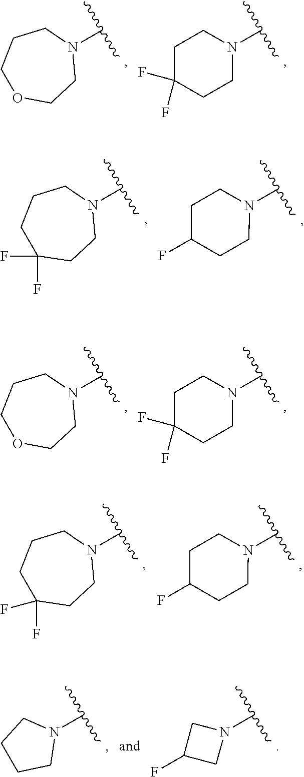

In some embodiments, R⁸ and R⁹ are methyl. In some embodiments, R⁸ is methyl, and R⁹ is ethyl.

In some embodiments, R¹ and R² are H; R³ is selected from H and C₁-C₆ optionally substituted alkyl; ring A is

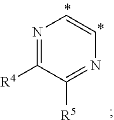

R⁴ and R⁵ are each independently selected from H, C₁₋₄haloalkyl, OC₁₋₄alkyl, OC₁₋₄haloalkyl, O(optionally substituted C₃₋₁₀ carbocycle), O(optionally substituted C₃₋₁₀ aryl), and N(R⁸R⁹); R⁸ and R⁹ are joined to form, together with the atom therebetween, a C₃₋₁₂heterocycloalkyl, optionally comprising one additional heteromoiety selected from N(R¹⁰), O, S, S(O) and SO₂, and optionally substituted with one or more substituents selected from halo, OH, NO₂, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), N(C₁₋₆alkyl)(C₁₋₆alkyl), SC₁₋₆alkyl, S(O)C₁₋₆alkyl, and SO₂C₁₋₆alkyl, wherein R¹⁰ is selected from C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, C(O)NH₂, C(O)NH(C₁₋₆alkyl), C(O)N(C₁₋₆alkyl)(C₁₋₆alkyl), S(O)C₁₋₆alkyl, and SO₂C₁₋₆alkyl.

In some embodiments, R¹ and R² are H; R³ is selected from H and C₁-C₆ optionally substituted alkyl; ring A is

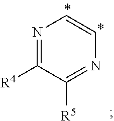

R⁴ and R⁵ are each independently selected from H, C₁₋₄haloalkyl, OC₁₋₄alkyl, OC₁₋₄haloalkyl, O(optionally substituted C₃₋₁₀ carbocycle), O(optionally substituted C₃₋₁₀ aryl), and N(R⁸R⁹); R⁸ and R⁹ are joined to form, together with the atom therebetween, a C₃₋₁₂heterocycloalkyl, optionally comprising one additional heteromoiety selected from N(R¹⁰) and O and optionally substituted with one or more substituents selected from halo, OH, NO₂, C₁₋₆alkyl, C₃₋₆cycloalkyl, aryl, C₅₋₆heteroaryl, C₃₋₆ heterocycloalkyl, C(O)C₁₋₆alkyl, OC₁₋₆alkyl, NH₂, and NH(C₁₋₆alkyl), wherein R¹⁰ is selected from C₁₋₆alkyl, C₃₋₆cycloalkyl, aryl, C₅-6heteroaryl, C₃₋₆ heterocycloalkyl, C(O)C₁₋₆alkyl, C(O)NH₂, and C(O)NH(C₁₋₆alkyl).

In some embodiments, R¹ and R² are H; R³ is selected from H and C₁-C₆ optionally substituted alkyl; ring A is

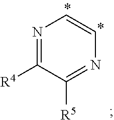

R⁴ and R⁵ are each independently selected from H, C₁₋₄haloalkyl, OC₁₋₄alkyl, and N(R⁸R⁹); R⁸ and R⁹ are joined to form, together with the atom therebetween, a C₃₋₁₂heterocycloalkyl, optionally comprising one additional oxygen atom and optionally substituted with one or more substituents selected from halo, OH, NO₂, C₁₋₆alkyl, aryl, C₅-6heteroaryl, C₃₋₆ heterocycloalkyl, OC₁₋₆alkyl, NH₂, and NH(C₁₋₆alkyl).

In some embodiments, R¹ and R² are H; R³ is selected from H and C₁-C₆ optionally substituted alkyl; ring A is

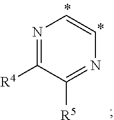

R⁴ and R⁵ are each independently selected from H, C₁₋₄haloalkyl, OC₁₋₄alkyl, and N(R⁸R⁹); R⁸ and R⁹ are joined to form, together with the atom therebetween, a C₃₋₁₂heterocycloalkyl, optionally comprising one additional oxygen atom and optionally substituted with one or more substituents selected from halo, OH, NO₂, C₁₋₆alkyl, aryl, and C₅-6heteroaryl.

In some embodiments, R¹ and R² are H; R³ is selected from H and C₁-C₆ optionally substituted alkyl; ring A is

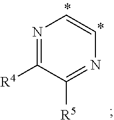

R⁴ and R⁵ are each independently selected from H and N(R⁸R⁹); R⁸ and R⁹ are joined to form, together with the atom therebetween, a C₃₋₁₂heterocycloalkyl, optionally comprising one additional oxygen atom and optionally substituted with one or more substituents selected from halo, OH, NO₂, C₁₋₆alkyl, aryl, and C₅₋₆heteroaryl.

In some embodiments, R¹ and R² are H; R³ is selected from H and C₁-C₆ optionally substituted alkyl; ring A is

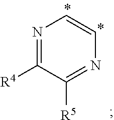

R⁴ and R⁵ are each independently selected from H and N(R⁸R⁹); R⁸ and R⁹ are joined to form, together with the atom therebetween, a C₃₋₁₂heterocycloalkyl, optionally comprising one additional oxygen atom and optionally substituted with one or more substituents selected from halo, OH, and C₁₋₆alkyl.

In some embodiments, R¹ and R² are H; R³ is selected from H and C₁-C₆ optionally substituted alkyl; ring A is

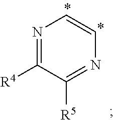

R⁴ and R⁵ are each independently selected from H and N(R⁸R⁹); R⁸ and R⁹ are joined to form, together with the atom therebetween, a C₃₋₁₂heterocycloalkyl, optionally comprising one additional oxygen atom and optionally substituted with one or more substituents selected from halo, and OH.

In some embodiments, R¹ and R² are H; R³ is selected from H and C₁-C₆ optionally substituted alkyl; ring A is

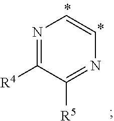

R⁴ and R⁵ are each independently selected from H and N(R⁸R⁹); R⁸ and R⁹ are joined to form, together with the atom therebetween, a C₃₋₁₂heterocycloalkyl, optionally comprising one additional oxygen atom and optionally substituted with one or more halo substituents.

In some embodiments, R¹ and R² are H; R³ is selected from H and C₁-C₆ optionally substituted alkyl; ring A is

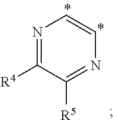

R⁴ and R⁵ are each independently selected from H and N(R⁸R⁹); R⁸ and R⁹ are joined to form, together with the atom therebetween, a C₃₋₁₂heterocycloalkyl, optionally comprising one additional oxygen atom and optionally substituted with one or more fluoro substituents.

In some embodiments, R¹ and R² are H; R³ is selected from H and C₁-C₆ optionally substituted alkyl; ring A is

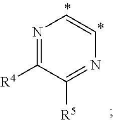

R⁴ and R⁵ are each independently selected from H, C₁₋₄haloalkyl, OC₁₋₄alkyl, OC₁₋₄haloalkyl, O(optionally substituted C₃₋₁₀ carbocycle), O(optionally substituted C₃₋₁₀ aryl), and N(R⁸R⁹); R⁸ and R⁹ are joined to form, together with the atom therebetween, a C₃₋₁₂heterocycloalkyl, optionally comprising one additional oxygen atom and optionally substituted with one or more substituents selected from halo, OH, NO₂, C₁₋₆alkyl, C₃₋₆cycloalkyl, aryl, C₅₋₆heteroaryl, C₃₋₆ heterocycloalkyl, C(O)C₁₋₆alkyl, OC₁₋₆alkyl, OC₁₋₆alkyleneOC₁₋₆alkyl, NH₂, and NH(C₁₋₆alkyl).

In some embodiments, the compound has the structure:

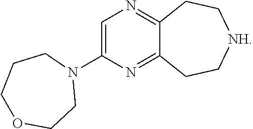 [SMILES: c1nc2c(nc1N1CCCOCC1)CCNCC2]

In some embodiments, the compound has the structure:

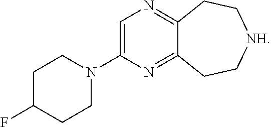 [SMILES: FC1CCN(c2cnc3c(n2)CCNCC3)CC1]

In some embodiments, the compound has the structure

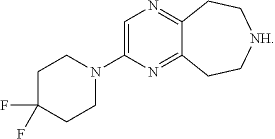 [SMILES: FC1(F)CCN(c2cnc3c(n2)CCNCC3)CC1]

In some embodiments, the compound has the structure:

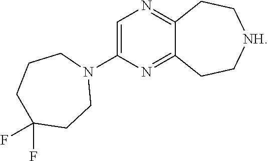 [SMILES: FC1(F)CCCN(c2cnc3c(n2)CCNCC3)CC1]

In some embodiments, the compound is provided in Table 1.

In an aspect, the present disclosure provides a pharmaceutical composition comprising a compound, or pharmaceutically acceptable salt or solvate thereof, as described in Formula (I) or Table 1 and a pharmaceutically acceptable excipient. In an aspect, the present disclosure provides a pharmaceutical composition comprising a compound, or pharmaceutically acceptable salt or solvate thereof, as described in any one of the preceding claims and a pharmaceutically acceptable excipient. In an aspect, the present disclosure provides a method comprising use of the composition of any of the preceding claims in the treatment of a disease or disorder mediated by the 5-HT2 receptor.

In an aspect, the present disclosure provides a method of use of the compound, salt, or solvate of any of the embodiments disclosed herein in the treatment of a disease or disorder mediated by the 5-HT2 receptor. In some embodiments, the disease or disorder is a 5-HT_{2A} and/or 5-HT2e receptor-mediated disorder. In some embodiments, the disease or disorder is depressive disorder, an anxiety disorder, panic attack, agoraphobia, specific phobia, social phobia, bipolar disorder, post-traumatic stress, an eating disorder, obesity, a gastro-intestinal disorder, alcoholism, drug addiction, schizophrenia, a psychotic disorder, a sleep disorder, sleep apnea, migraine, sexual dysfunction, a central nervous system disorder, trauma, stroke, spinal cord injury, a cardio-vascular disorder, diabetes insipidus, or obsessive disorder.

In an aspect, the present disclosure provides a method of use of the composition of any of the preceding claims to ameliorate at least one symptom of a brain disorder, stress, anxiety, addiction, depression, compulsive behavior, or by promoting weight loss, or by improving mood, or by treating or preventing a psychological disorder, or by enhancing performance. In an aspect, the present disclosure provides a method of treating at least one symptom of a brain disorder, stress, anxiety, addiction, depression, or compulsive behavior comprising administering to a patient in need thereof the compound, salt, or solvate of any of the embodiments disclosed herein. In an aspect, the present disclosure provides a method of promoting weight loss comprising administering to a patient in need thereof the compound, salt, or solvate of any of the embodiments disclosed herein. In an aspect, the present disclosure provides a method of improving mood comprising administering to a patient in need thereof the compound, salt, or solvate of any of the embodiments disclosed herein. In an aspect, the present disclosure provides a method of preventing a psychological disorder comprising administering to a patient in need thereof the compound, salt, or solvate of any of the embodiments disclosed herein. In an aspect, the present disclosure provides a method of enhancing performance comprising administering to a patient in need thereof the compound, salt, or solvate of any of the embodiments disclosed herein. In an aspect, the present disclosure provides a method of treating depressive disorder, an anxiety disorder, panic attack, agoraphobia, specific phobia, social phobia, bipolar disorder, post-traumatic stress, an eating disorder, obesity, a gastro-intestinal disorder, alcoholism, drug addiction, schizophrenia, a psychotic disorder, a sleep disorder, sleep apnea, migraine, sexual dysfunction, a central nervous system disorder, trauma, stroke, spinal cord injury, a cardio-vascular disorder, diabetes insipidus, or obsessive disorder comprising administering to a patient in need thereof the compound, salt, or solvate of any of the embodiments disclosed herein.

Preparation of Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Comwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Comwall, U.K.), Lancaster Synthesis (Windham, NH), Maybridge Chemical Co. Ltd. (Comwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modem Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modem Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

The compounds of Formula (I) generally can be prepared according to the processes illustrated in the Schemes below. In the structural formulae shown below, the variables are as defined in Formula (I) unless otherwise stated.

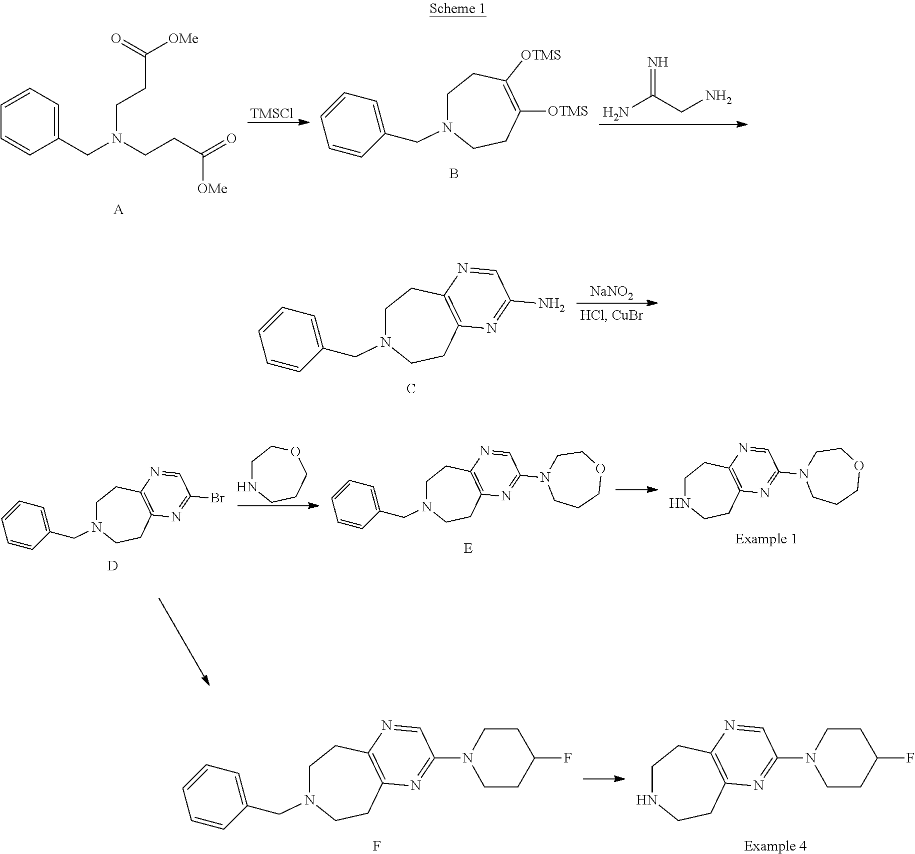

The required tetrahydro-4,5-bis(trimethylsilyloxy)-azepines B may be obtained by acyloin condensation from dicarboxylic acid diester A (commercial from Ambeed, Frontier, JW Labs and Comiblock) with sodium in the presence of chlorotrimethylsilane [see J. Org. Chem. 1977, 42, (24), 3393 or Synthesis 263 (1971)]. Cyclocondensation of aminoacetamidine hydrochloride with B affords pyrazine C. Diazotization followed by CuBr provides intermediate D. Displacement of the bromine with an amine at 145° C. followed by hydrogenolysis affords the target compounds.

An alternative synthetic path to compounds of Formula (I) wherein Ring A is a substituted pyrazine proceeds according to Scheme 2.

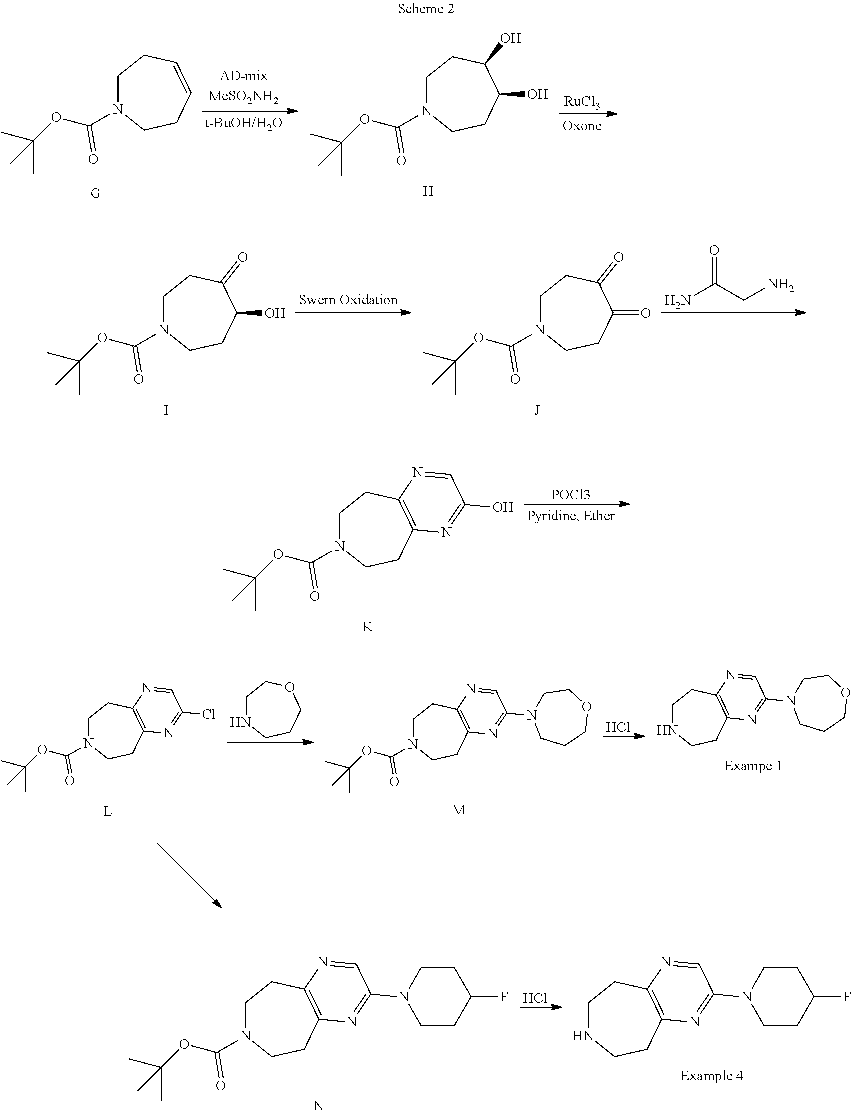

Azepine derivative G can be dihydroxylated to intermediate diol H. Initial mono-oxidation with RuCl3/Oxone to I followed by Swern oxidation provides diketone J. Treatment of the diketone J under basic conditions with glycine amide leads to the prazine heterocycle K. Subsequent chlorination of K with POCl₃ gives the versatile intermediate L. Coupling of L with various amines follow by Boc deprotection afforded the compounds of Formula (I).

To prepare the compounds of Formula (I) wherein Ring A is a substituted pyrazine involves transforming intermediate L to the TBS-protected derivative O (Scheme 3). Compound O is then alkylated to afford ketone P (Bioorganic & Medicinal Chemistry Letters 2006, 16(12), 3302-3305). Silyl deprotection followed by oxidation gives the alpha-methyl diketone which can be transformed to compounds of Formula (I) according to Scheme 2.

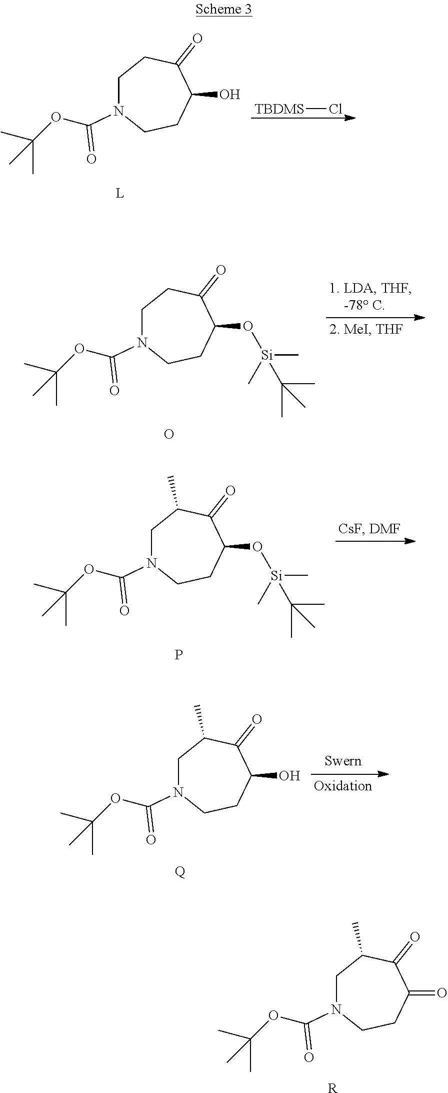

Compounds of Formula (I) wherein Ring A is the substituted pyrrolopyrazine can be prepared according to Scheme 4.

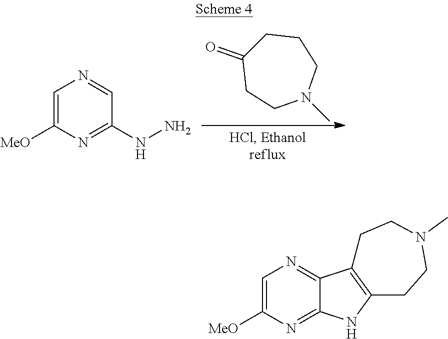

Compounds of Formula (I) wherein Ring A is the substituted pyrrolopyrazine can be prepared according to Scheme 5.

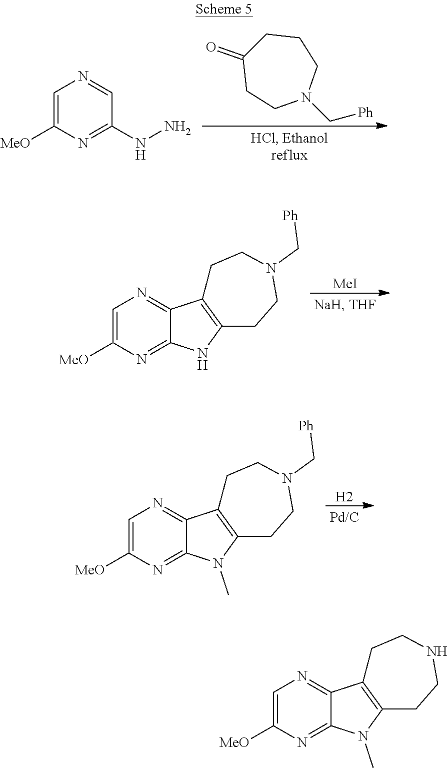

Generally, the reactions described above are performed in a suitable inert organic solvent and at temperatures and for times that will optimize the yield of the desired compounds. Examples of suitable inert organic solvents include, but are not limited to, dimethylformamide (DMF), dioxane, methylene chloride, chloroform, tetrahydrofuran (THF), toluene, and the like.

Pharmaceutical Compositions

In certain embodiments, the heterocyclic 5-HT₂ₐ and/or 5-HT_{2c} receptor agonists compound described herein is administered as a pure chemical. In other embodiments, the heterocyclic 5-HT2a and/or 5-HT_{2c} receptor agonists compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005).

Provided herein is a pharmaceutical composition comprising at least one heterocyclic 5-HT₂ₐ and/or 5-HT_{2c} receptor agonists compound as described herein, or a stereoisomer, pharmaceutically acceptable salt, hydrate, or solvate thereof, together with one or more pharmaceutically acceptable carriers. The carrier(s) (or excipient(s) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I), or a pharmaceutically acceptable salt or solvate thereof.

One embodiment provides a method of preparing a pharmaceutical composition comprising mixing a compound of Formula (I), or a pharmaceutically acceptable salt or solvate thereof, and a pharmaceutically acceptable carrier.

In certain embodiments, the heterocyclic 5-HT₂ₐ and/or 5-HT_{2c} receptor agonists compound as described by Formula (I), or a pharmaceutically acceptable salt or solvate thereof, is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005).

In some embodiments, the heterocyclic 5-HT₂ₐ and/or 5-HT_{2c} receptor agonists compound as described by Formula (I), or pharmaceutically acceptable salt or solvate thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one heterocyclic 5-HT₂ₐ and/or 5-HT_{2c} receptor agonists compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

Methods of Treatment

One embodiment provides a compound of Formula (I), or a pharmaceutically acceptable salt or solvate thereof, for use in a method of treatment of the human or animal body.

One embodiment provides a compound of Formula (I), or a pharmaceutically acceptable salt or solvate thereof, for use in a method of treatment of a disease or disorder mediated by the 5-HT_{2A} and/or 5-HT_{2c} receptor. In some embodiments, the disease or disorder is mediated by activating the 5-HT_{2A} and/or 5-HT_{2c} receptor signaling axis. In some embodiments, the disease, disorder or condition that is treatable by activating the 5-HT_{2A} and/or 5HT2C receptor, is a CNS disorder. In some embodiments, the treatment comprises administration of an amount of at least one compounds described herein that is effective to ameliorate at least one symptom of a brain disorder, for example, improvement in mental or physical well-being in the subject (e.g., by treating stress, anxiety, addiction, depression, compulsive behavior, by promoting weight loss, by improving mood, by treating or preventing a condition (e.g., psychological disorder), or by enhancing performance.

A “5-HT_{2A} and/or 5-HT_{2c} receptor-mediated disorder”, as used herein, is a disorder in which there is believed to be involvement of the pathway controlled by the 5-HT_{2A} and/or 5-HT2e receptor and which is ameliorated by treatment with an agonist of the 5-HT_{2A} and/or 5-HT2e receptor. 5-HT_{2A} and/or 5-HT_{2c} receptor-mediated disorders include a depressive disorder, an anxiety disorder, including panic attack, agoraphobia, and specific or social phobia, bipolar disorder, post-traumatic stress, an eating disorder, obesity, a gastro-intestinal disorder, alcoholism, drug addiction, schizophrenia, a psychotic disorder, a sleep disorder, including sleep apnea, migraine, sexual dysfunction, a central nervous system disorder, including trauma, stroke and spinal cord injury, a cardio-vascular disorder, diabetes insipidus, or obsessive disorder.

Provided herein is the method wherein the pharmaceutical composition is administered orally. Provided herein is the method wherein the pharmaceutical composition is administered by injection.

Other embodiments and uses will be apparent to one skilled in the art in light of the present disclosures. The following examples are provided merely as illustrative of various embodiments and shall not be construed to limit the invention in any way.

### EXAMPLES

I. Chemical Synthesis

The compounds of Formula (I) generally can be prepared according to the processes illustrated below. In the structural formulae shown below the variables are as defined in Formula (I) unless otherwise stated.

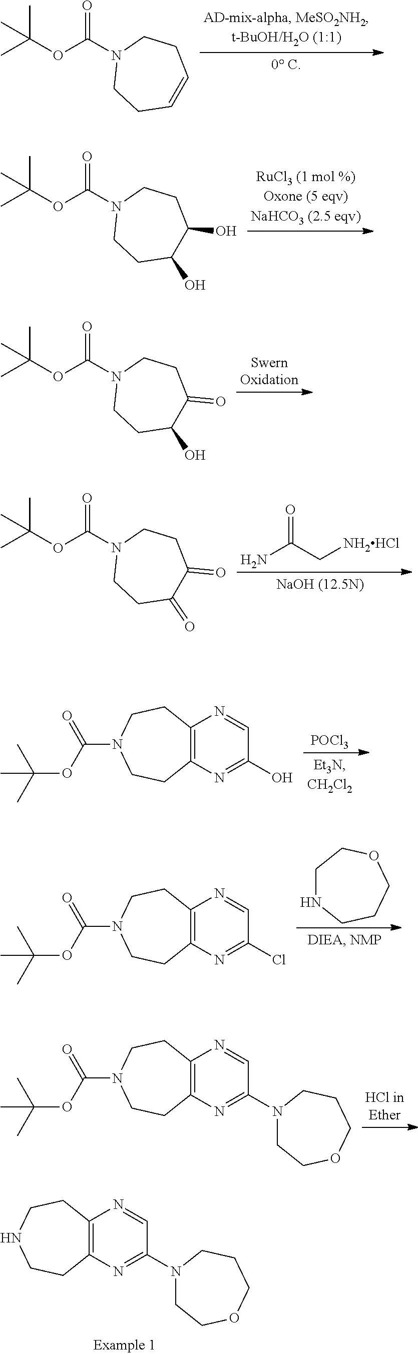

### Example 1: Synthesis of 4-(6,7,8,9-tetrahydro-5H-pyrazino[2,3-d]azepin-2-yl)-1,4-oxazepane (e.g., 4-{5H,6H,7H,8H,9H-pyrazino[2,3-d]azepin-2-yl}-1,4-oxazepane)

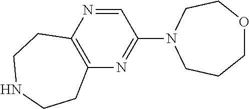 [SMILES: c1nc2c(nc1N1CCCOCC1)CCNCC2]

### Step 1: Synthesis tert-butyl (4R,5S)-4,5-dihydroxyazepane-1-carboxylate

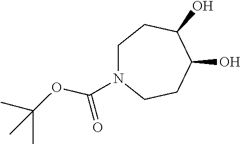 [SMILES: CC(C)(C)OC(=O)N1CC[C@@H](O)[C@@H](O)CC1]

A 50 mL round-bottomed flask equipped with a magnetic stirring bar was charged with 5.6 g AD-mix-α and methanesulfonylamide (380 mg, 4 mmol). tert-Butanol (20 mL) and water (20 mL) was added and the slurry was stirred at room temperature until all solids dissolved. The orange solution was cooled to 0° C. The olefin (4 mmol) was added in one portion. The mixture was kept at 0° C. with vigorous stirring until TLC showed complete conversion. Sat. Na₂SO₃-solution (20 mL) was added and the reaction was allowed to warm up within 1 h. Phases were separated, the aqueous layer was extracted with ethyl acetate (3×40 mL). The combined organic layer was dried over Na₂SO₄ and concentrated in vacuum. The resulting crude product was further purified via flash-chromatography to afford a colorless oil.

### Step 2: Synthesis of tert-butyl (S)-4-hydroxy-5-oxoazepane-1-carboxylate

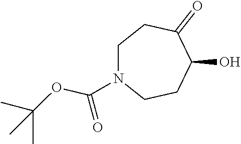 [SMILES: CC(C)(C)OC(=O)N1CCC(=O)[C@@H](O)CC1]

In a 100-mL round-bottomed flask equipped with magnetic stirring bar and overpressure valve was charged with NaHCO₃ (420 mg, 5.0 mmol). A 0.1 M aqueous solution of RuCl₃ (200 μL, 0.02 mmol) was added and the suspension was diluted with 1.8 mL H₂O, 12 mL CH₃CN and 12 mL ethyl acetate. Oxone (6.14 g, 10 mmol) was added in one portion to the resulting brownish suspension (gas evolution!). When the color turned bright yellow, the diol (2 mmol) was added in one portion. The reaction was followed by TLC. After complete conversion the mixture was poured onto 30 mL sat. NaHCO₃ and 30 mL sat. Na₂SO₃ solution. Phases were separated and the aqueous layer was extracted with ethyl acetate (3×30 mL). After drying the combined organic layer over Na₂SO₄ and evaporation of the solvent in vacuum the oily crude product was purified by flash chromatography to afford a colouless oil.

### Step 3: Synthesis of tert-butyl (S)-4-hydroxy-5-oxoazepane-1-carboxylate

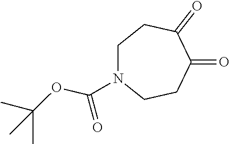 [SMILES: CC(C)(C)OC(=O)N1CCC(=O)C(=O)CC1]

A solution of DMSO (164 mg, 2.1 mmol) in CH₂Cl₂ (1 mL) was added dropwise to a solution of oxalyl chloride (133 mg, 1.05 mmol) in CH₂Cl₂ (2 mL) at −78° C. The resulting suspension was stirred for 15 min. and a solution of the crude tert-butyl (S)-4-hydroxy-5-oxoazepane-1-carboxylate in CH₂Cl₂ (3 mL) was added to the reaction mixture. The suspension was stirred at −78° C. for 15 min. Then Et₃N (319 mg, 3.15 mmol) was slowly added. The suspension was warmed up to 0° C. and stirred for additional 4 h. The mixture was diluted with CH₂Cl₂ (10 mL) and water (10 mL) was added. The layers were separated, and the aqueous phase was extracted with CH₂Cl₂ (3×5 mL). The combined organic portions were washed with brine, dried over Na₂SO₄, and concentrated under reduced pressure. The residue was purified by silica gel column chromatography (gradient hexane/EtOAc 19:1) to give desired diketone as a light-yellow solid

### Step 4: Synthesis of tert-butyl 2-hydroxy-5,6,8,9-tetrahydro-7H-pyrazino[2,3-d]azepine-7-carboxylate

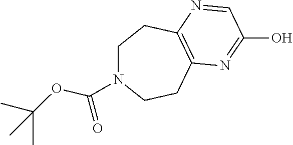 [SMILES: CC(C)(C)OC(=O)N1CCc2ncc(O)nc2CC1]

A solution of diketone (2.54 g) in 4 ml of methanol was added dropwise to a cold (−10° C.) solution of glycinamide hydrochloride (2.5 g) in 4 mL of water in a three-neck, round-bottom flask equipped with a stirrer. 12.5 N sodium hydroxide-water solution (1 mL) was then added dropwise. (It was observed that, if the temperature rose above 0° C. during these additions, yield of product was drastically reduced.) The reaction mixture was stored at −10° C. for 12 h, after which hydrochloric acid, Sp. Gr. 1.18, was added to the solution in 0.5 mL portions until the solution was slightly acid to litmus paper. The precipitate was collected by filtration with suction and rinsed with ethyl ether to give yellow crude product (25.4 g), which was purified by recrystallization to afford the desired product as a white solid (2.21 g). LCMS [M+H]⁺ 266.

### Step 5: Synthesis of tert-butyl 2-chloro-5,6,8,9-tetrahydro-7H-pyrazino[2,3-d]azepine-7-carboxylate

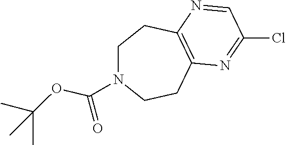 [SMILES: CC(C)(C)OC(=O)N1CCc2ncc(Cl)nc2CC1]

A stirred solution of tert-butyl 2-hydroxy-5,6,8,9-tetrahydro-7H-pyrazino[2,3-d]azepine-7-carboxylate (430 mg, 1.7 mmol) in toluene (10 mL) was added Et₃N (10 eqv) followed by POCl₃ (5.2 mL) in an ice bath. The mixture was then heated to 100° C. for 4 hours. The orange solution was cooled to room temperature and stirred rapidly in a mixture of CH₂Cl₂ (100 mL) and ice cold 10% Na₂CO₃ (100 mL) for 15 minutes. The organic layer was isolated and washed 2×100 mL with 10% Na₂CO₃. The organics were isolated, dried (MgSO₄), filtered and concentrated to the title compound as an off white solid. LCMS [M+H]⁺ 284.

### Step 6: Synthesis of tert-butyl 2-(1,4-oxazepan-4-yl)-5,6,8,9-tetrahydro-7H-pyrazino[2,3-d]azepine-7-carboxylate

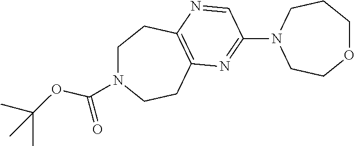 [SMILES: CC(C)(C)OC(=O)N1CCc2ncc(N3CCCOCC3)nc2CC1]

To a stirred solution of tert-butyl 2-chloro-5,6,8,9-tetrahydro-7H-pyrazino[2,3-d]azepine-7-carboxylate (0.113 g, 0.40 mmol) in NMP (2 mL) was added homomorpholine (1.5 eq) and DIPEA (3 eq) at room temperature in a microwave reactor. The solution was heated at 130° C. for 30 min under microwave irradiation. After cooling, the solvent was removed in vaccuo and the remaining solid was taken up in ethyl acetate and washed with water. Drying with Na₂SO₄, filtration and evaporation. Yield 80 mg (60%) as a solid. LCMS [M+H]⁺ 349.

### Step 7

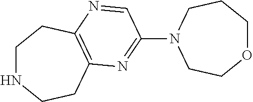 [SMILES: c1nc2c(nc1N1CCCOCC1)CCNCC2]

To a stirred solution of tert-butyl 2-(1,4-oxazepan-4-yl)-5,6,8,9-tetrahydro-7H-pyrazino[2,3-d]azepine-7-carboxylate (0.080 g) in ether was added 1 N HCl in ether (2 mL) and the mixture was stirred at room temperature for 2 h. The mixture was filtered to give 4-{5H,6H,7H,8H,9H-pyrazino[2,3-d]azepin-2-yl}-1,4-oxazepane as an HCl salt. Yield 40 mg as a solid. LCMS [M+H]⁺ 249.

### Example 1: Alternative Synthesis of 4-(6,7,8,9-tetrahydro-5H-pyrazino[2,3-d]azepin-2-yl)-1,4-oxazepane (Procedure A)

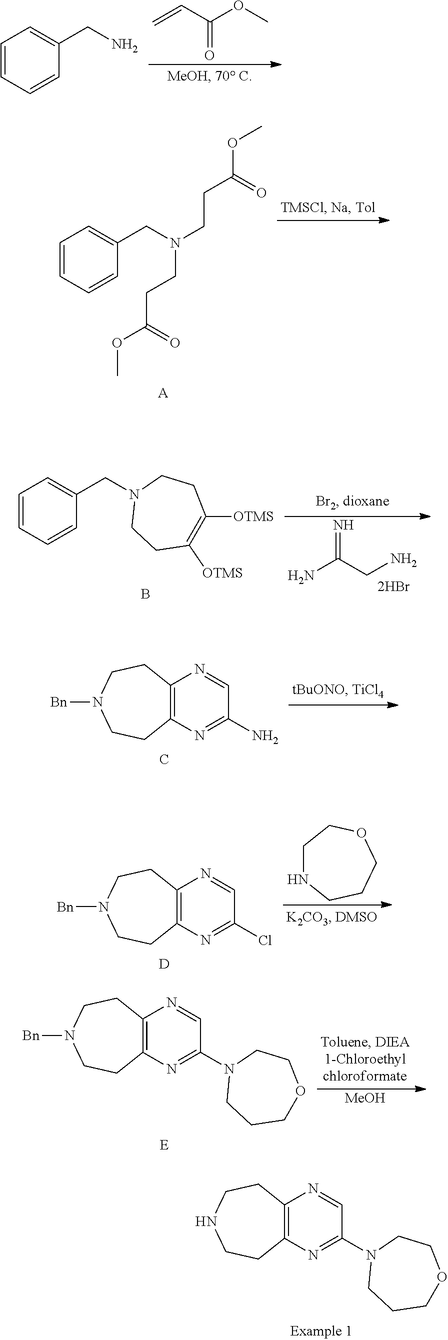

### Step 1: Synthesis of dimethyl 3,3′-(benzylazanediyl)dipropionate

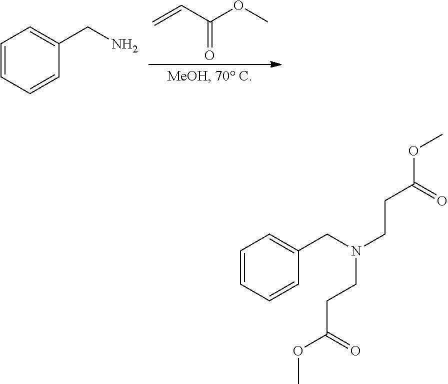

A solution of benzylamine (10 g, 93.32 mmol, 1 equiv) and methyl acrylate (16.07 g, 186.64 mmol, 2 equiv) in MeOH (50 mL) was stirred for 2 h at 70° C. The resulting mixture was concentrated under reduced pressure. The residue was purified by silica gel colunn chromatography, eluted with PE/EA (1:1) to afford methyl 3-[benzyl(3-methoxy-3-oxopropyl)amino]propanoate (20 g, 76.72%) as a colorless oil. LCMS (ESI): [M+H]⁺=280.1.

### Step 2: Synthesis of 1-benzyl-4,5-bis(trimethylsilyl)oxy)-2,3,6,7-tetrahydro-1H-azepine

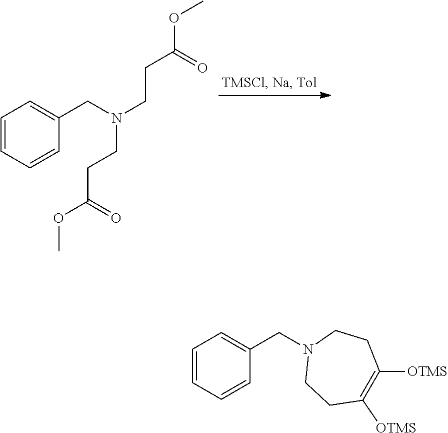

To a stirred mixture of Na (6.91 g, 300.56 mmol, 4.20 equiv) in toluene were added TMSCl (35.78 g, 329.35 mmol, 4.6 equiv) and methyl 3-[benzyl(3-methoxy-3-oxopropyl)amino]propanoate (20 g, 71.59 mmol, 1 equiv) in portions at 110° C. under nitrogen atmosphere. The final reaction mixture was heated for 2 h at 110° C. The mixture was allowed to cool down to room temperature. The resulting mixture was concentrated under reduced pressure. The resulting mixture was washed with 80 ml of aqueous NaHCO₃ dried with Na₂SO₄, and concentrated under reduced pressure. The residue was purified by silica gel column chromatography, eluted with PE/EA (12:1) to afford 1-benzyl-4,5-bis[(trimethylsilyl)oxy]-2,3,6,7-tetrahydroazepine (6 g, 23.04%) as a light-yellow oil. LCMS (ESI): [M+H]⁺=364.2.

### Step 3: Synthesis of 7-benzyl-6,7,8,9-tetrahydro-5H-pyrazino[2,3-d]azepin-2-amine

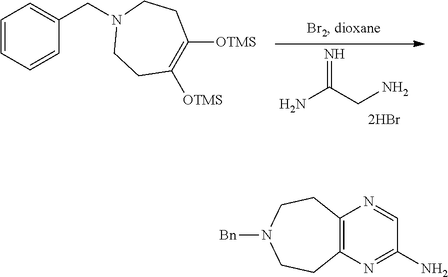

To a stirred solution of 1-benzyl-4,5-bis[(trimethylsilyl)oxy]-2,3,6,7-tetrahydroazepine (6 g, 16.49 mmol, 1 equiv) in 1,4-dioxane was added Br2 (2.64 g, 16.49 mmol, 1 equiv) dropwise at 0° C. under nitrogen atmosphere. Pyridine (5.22 g, 65.99 mmol, 4 equiv) was added dropwise at 0° C. under nitrogen atmosphere after 0.5 h. Then, 2-aminoethanimidamide (1.93 g, 26.39 mmol, 1.6 equiv) in 1,4-dioxane was added dropwise at 0° C. under nitrogen. The resulting mixture was stirred for overnight at room temperature under nitrogen atmosphere. The residue mixture was basified to pH 10 with aq. NaOH (2M). The resulting mixture was concentrated under reduced pressure. The residue was purified by silica gel column chromatography, eluted with CH₂Cl₂/MeOH (1:1) to afford 7-benzyl-5H,6H,8H,9H-pyrazino[2,3-d]azepin-2-amine (2.5 g, 59.57%) as a brown solid. LCMS (ESI): [M+H]⁺=255.2.

### Step 4: Synthesis of 7-benzyl-2-chloro-6,7,8,9-tetrahydro-5H-pyrazino[2,3-d]azepine

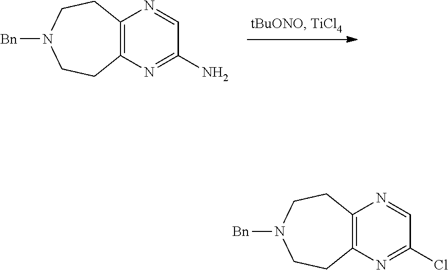 [SMILES: Nc1cnc2c(n1)CCN(Cc1ccccc1)CC2 ; Clc1cnc2c(n1)CCN(Cc1ccccc1)CC2]

To a stirred solution of 7-benzyl-5H,6H,8H,9H-pyrazino[2,3-d]azepin-2-amine (1.5 g, 5.89 mmol, 1 equiv) in CH₂Cl₂ were added TiCl₄ (2.68 g, 14.15 mmol, 2.4 equiv) and t-BuONO (1.22 g, 11.79 mmol, 2 equiv) dropwise at 0° C. The final reaction mixture was stirred for 1 h at room temperature. The mixture was basified to pH 8 with aq. NaOH (2M). The resulting mixture was extracted with CH₂Cl₂ (3×20 mL) and dried over anhydrous Na₂SO₄. After filtration, the filtrate was concentrated under reduced pressure. The residue was purified by silica gel column chromatography, eluted with CH₂Cl₂/MeOH (10:1) to afford 7-benzyl-2-chloro-5H,6H,8H,9H-pyrazino[2,3-d]azepine (800 mg, 49.55%) as a light yellow solid. LCMS (ESI): [M+H]⁺=274.1

### Step 5: Synthesis of 4-(7-benzyl-6,7,8,9-tetrahydro-5H-pyrazino[2,3-d]azepin-2-yl)-1,4-oxazepane

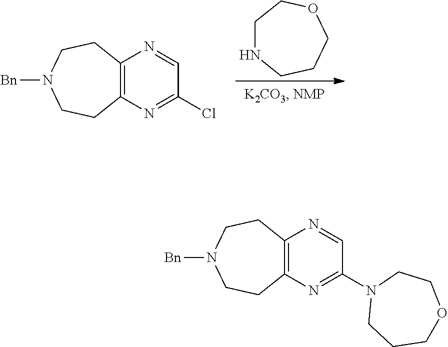 [SMILES: Clc1cnc2c(n1)CCN(Cc1ccccc1)CC2 ; C1CNCCOC1 ; c1ccc(CN2CCc3ncc(N4CCCOCC4)nc3CC2)cc1]

Into a 20 mL sealed tube were added 7-benzyl-2-chloro-5H,6H,8H,9H-pyrazino[2,3-d]azepine (800 mg, 2.92 mmol, 1 equiv), 1,4-oxazepane (1.61 g, 11.68 mmol, 4 equiv), K₂CO₃ (4.7 g, 14.61 mmol, 5 equiv), and NMP (10 mL). The final reaction mixture was stirred for 5 h at 150° C. The mixture was allowed to cool down to room temperature. The resulting mixture was quenched with water (20 mL), extracted with ethyl acetate (EA) (3×20 mL), and dried over anhydrous Na₂SO₄. After filtration, the filtrate was concentrated under reduced pressure. The residue was purified by silica gel column chromatography, eluted with PE/EA (1:1) to afford 4-{7-benzyl-5H,6H,8H,9H-pyrazino[2,3-d]azepin-2-yl}-1,4-oxazepane (400 mg, 40.44%) as a light yellow oil. LCMS (ESI): [M+H]⁺=339.2.

### Step 6: Synthesis of 4-{5H,6H,7H,8H,9H-pyrazino[2,3-d]azepin-2-yl}-1,4-oxazepane

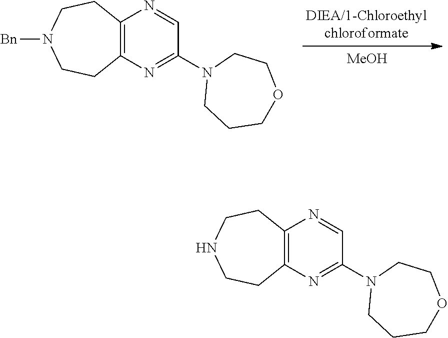 [SMILES: "MeOH" ; c1ccc(CN2CCc3ncc(N4CCCOCC4)nc3CC2)cc1 ; c1nc2c(nc1N1CCCOCC1)CCNCC2]

To a stirred solution/mixture of 4-{7-benzyl-5H,6H,8H,9H-pyrazino[2,3-d]azepin-2-yl}-1,4-oxazepane (400 mg, 1.18 mmol, 1 equiv) in toluene were added DIEA (1.22 g, 9.45 mmol, 8 equiv) and 1-chloroethyl chloroformate (1.35 g, 9.45 mmol, 8 equiv) at room temperature. The final reaction mixture was stirred for 2 h at 100° C. The mixture was allowed to cool down to room temperature. The resulting mixture was concentrated under reduced pressure. Then MeOH (5 mL) was added, and the mixture was heated for 1 h at 80° C. The mixture was allowed to cool down to room temperature. The resulting mixture was concentrated under reduced pressure. The crude product was purified by prep-HPLC to afford 4-{5H,6H,7H,8H,9H-pyrazino[2,3-d]azepin-2-yl}-1,4-oxazepane (78.3 mg, 26.60%) as a yellow oil. LCMS (ESI): [M+H]⁺=249.2. ¹H NMR (400 MHz, DMSO-d6) δ 7.78 (s, 1H), 3.80-3.64 (m, 6H), 3.60 (t, J=5.5 Hz, 2H), 2.87-2.72 (m, 8H), 1.86 (p, J=5.8 Hz, 2H).

### Example 4: Synthesis of 4-fluoro-1-{5H,6H,7H,8H,9H-pyrazino[2,3-d]azepin-2-yl}piperidine

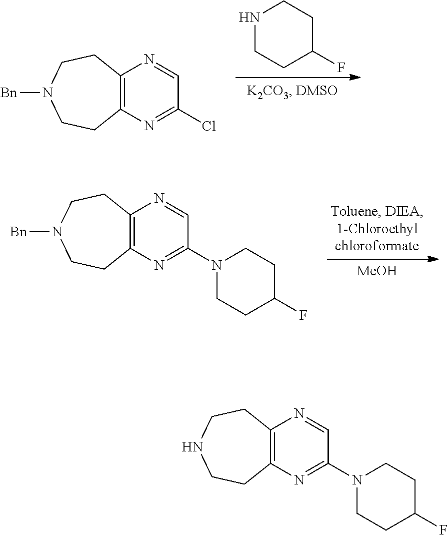 [SMILES: "MeOH" ; Clc1cnc2c(n1)CCN(Cc1ccccc1)CC2 ; FC1CCNCC1 ; FC1CCN(c2cnc3c(n2)CCN(Cc2ccccc2)CC3)CC1 ; FC1CCN(c2cnc3c(n2)CCNCC3)CC1]

### Step 1: Synthesis of 1-{7-benzyl-5H,6H,8H,9H-pyrazino[2,3-d]azepin-2-yl}-4-fluoropiperidine

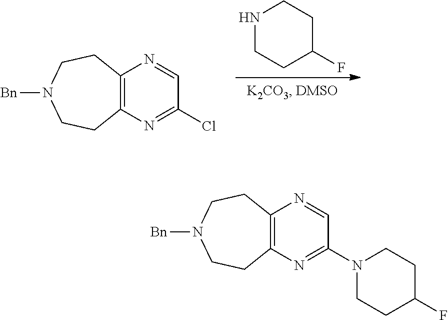 [SMILES: Clc1cnc2c(n1)CCN(Cc1ccccc1)CC2 ; FC1CCNCC1 ; FC1CCN(c2cnc3c(n2)CCN(Cc2ccccc2)CC3)CC1]

Into a 10 mL sealed tube were added 7-benzyl-2-bromo-5H,6H,8H,9H-pyrazino[2,3-d]azepine (500 mg, 1.57 mmol, 1 equiv), 4-fluoropiperidine (440 mg, 3.14 mmol, 2 equiv), and K₂CO₃ (2.55 g, 7.85 mmol, 5 equiv) with DMSO (5 mL). The final reaction mixture was heated for 5 h at 150° C. The mixture was allowed to cool down to room temperature. The resulting mixture was quenched with water (20 mL), extracted with EA (3×20 mL), and dried over anhydrous Na₂SO₄. After filtration, the filtrate was concentrated under reduced pressure. The residue was purified by silica gel column chromatography, eluted with PE/EA (1:1) to afford 1-{7-benzyl-5H,6H,8H,9H-pyrazino[2,3-d]azepin-2-yl}-4-fluoropiperidine (300 mg, 56.08%) as a light yellow oil. LCMS (ESI): [M+H]⁺=341.2

### Step 2: Synthesis of 4-fluoro-1-{5H,6H,7H,8H,9H-pyrazino[2,3-d]azepin-2-yl}piperidine

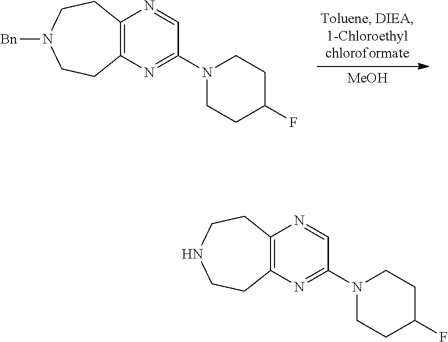 [SMILES: "MeOH" ; FC1CCN(c2cnc3c(n2)CCN(Cc2ccccc2)CC3)CC1 ; FC1CCN(c2cnc3c(n2)CCNCC3)CC1]

To a stirred solution of 1-{7-benzyl-5H,6H,8H,9H-pyrazino[2,3-d]azepin-2-yl}-4-fluoropiperidine (300 mg, 0.88 mmol, 1 equiv) in toluene were added DIEA (911 mg, 7.04 mmol, 8 equiv) and 1-chloroethyl chloroformate (1.00 g, 7.04 mmol, 8 equiv) at room temperature. The final reaction mixture was heated for 2 h at 100° C. The mixture was allowed to cool down to room temperature. The resulting mixture was concentrated under reduced pressure. Then, MeOH (5 mL) was added, and the crude reaction mixture was heated for 1 h at 80° C. The mixture was allowed to cool down to room temperature. The resulting mixture was concentrated under reduced pressure. The crude product was purified by prep-HPLC to afford 4-fluoro-1-{5H,6H,7H,8H,9H-pyrazino[2,3-d]azepin-2-yl}piperidine (56 mg, 25.250%) as ayellow solid. LCMS (ESI): [M+H]+=251.2. ¹H NMR (400 MHz, DMSO-d6) δ 7.97 (s, 1H), 4.95-4.79 (in, 1H), 3.77-3.59 (m, 2H), 3.49-3.42 (m, 2H), 2.99-2.74 (m, 8H), 1.94-1.88 (m, 2H), 1.73-1.67 (in, 2H).

The compounds in Table 2 were prepared in a similar manner.

TABLE 2

| Ex- | | |
|---|---|---|
| ample | Compound Structure | Product Mass |
| 2 | 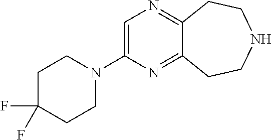 [SMILES: FC1(F)CCN(c2cnc3c(n2)CCNCC3)CC1] | LCMS [M + H]⁺ 269 |
| 3 | 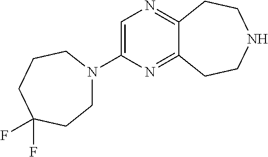 [SMILES: FC1(F)CCCN(c2cnc3c(n2)CCNCC3)CC1] | LCMS [M + H]⁺ 283 |
| 15 | 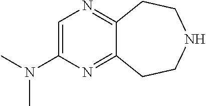 | LCMS [M + H]⁺ 192 |
| 16 | 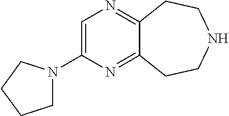 [SMILES: c1nc2c(nc1N1CCCC1)CCNCC2] | LCMS [M + H]⁺ 219 |
| 17 | 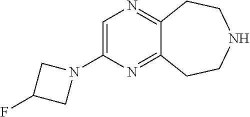 [SMILES: FC1CN(c2cnc3c(n2)CCNCC3)C1] | LCMS [M + H]⁺ 223 |
| 24 | 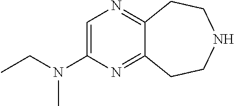 | LCMS [M + H]⁺ 207 |
| 5 | 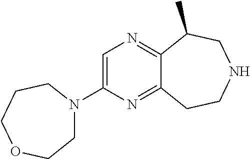 [SMILES: C[C@H]1CNCCc2nc(N3CCCOCC3)cnc21] | LCMS [M + H]⁺ 263 |
| 6 | 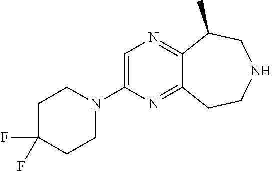 [SMILES: C[C@H]1CNCCc2nc(N3CCC(F)(F)CC3)cnc21] | LCMS [M + H]⁺ 283 |
| 7 | 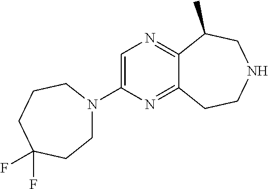 [SMILES: C[C@H]1CNCCc2nc(N3CCCC(F)(F)CC3)cnc21] | LCMS [M + H]⁺ 297 |
| 8 | 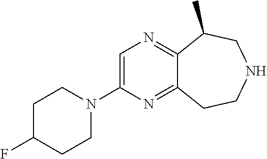 [SMILES: C[C@H]1CNCCc2nc(N3CCC(F)CC3)cnc21] | LCMS [M + H]⁺ 265 |

### Example 21: Synthesis of 2-phenoxy-6,7,8,9-tetrahydro-5H-pyrazino[2,3-d]azepine

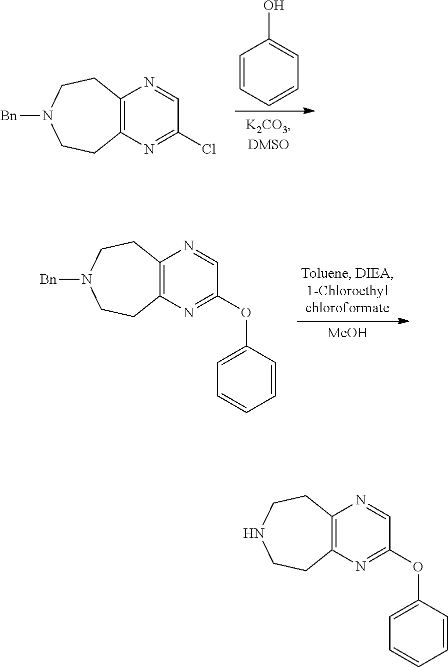 [SMILES: "MeOH" ; Clc1cnc2c(n1)CCN(Cc1ccccc1)CC2 ; Oc1ccccc1 ; c1ccc(CN2CCc3ncc(Oc4ccccc4)nc3CC2)cc1 ; c1ccc(Oc2cnc3c(n2)CCNCC3)cc1]

### Step 1: Synthesis of 1-{7-benzyl-5H,6H,8H,9H-pyrazino[2,3-d]azepin-2-yl}-4-fluoropiperidine

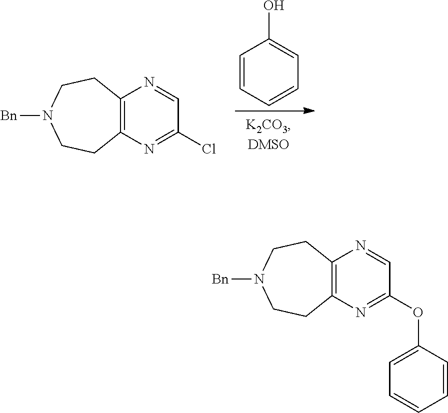 [SMILES: Clc1cnc2c(n1)CCN(Cc1ccccc1)CC2 ; Oc1ccccc1 ; c1ccc(CN2CCc3ncc(Oc4ccccc4)nc3CC2)cc1]

Into a 10 mL sealed tube were added 7-benzyl-2-chloro-5H,6H,8H,9H-pyrazino[2,3-d]azepine (500 mg, 1.57 mmol, 1 equiv), phenol (440 mg, 3.14 mmol, 2 equiv), and K₂CO₃ (2.55 g, 7.85 mmol, 5 equiv) with DMSO (5 mL). The final reaction mixture was heated for 5 h at 150′° C. The mixture was allowed to cool down to room temperature. The resulting mixture was quenched with water (20 mL), extracted with EA (3×20 mL), and dried over anhydrous Na₂SO₄. After filtration, the filtrate was concentrated under reduced pressure. The residue was purified by silica gel column chromatography, eluted with PE/EA (1:1) to afford 7-benzyl-2-phenoxy-6,7,8,9-tetrahydro-5H-pyrazino[2,3-d]azepine (300 mg, 56.080%) as a light yellow oil.

LCMS (ESI): [M+H]+=332.

### Step 2: Synthesis of 2-phenoxy-6,7,8,9-tetrahydro-5H-pyrazino[2,3-d]azepine

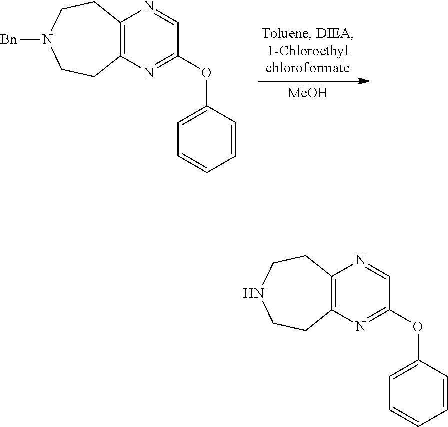 [SMILES: "MeOH" ; c1ccc(CN2CCc3ncc(Oc4ccccc4)nc3CC2)cc1 ; c1ccc(Oc2cnc3c(n2)CCNCC3)cc1]

To a stirred solution of 7-benzyl-2-phenoxy-6,7,8,9-tetrahydro-5H-pyrazino[2,3-d]azepine (300 mg, 0.88 mmol, 1 equiv) in toluene were added DIEA (911 mg, 7.04 mmol, 8 equiv) and 1-chloroethyl chloroformate (1.00 g, 7.04 mmol, 8 equiv) at room temperature. The final reaction mixture was heated for 2 h at 100° C. The mixture was allowed to cool down to room temperature. The resulting mixture was concentrated under reduced pressure. Then, MeOH (5 mL) was added, and the reaction mixture was heated for 1 h at 80° C. The mixture was allowed to cool down to room temperature. The resulting mixture was concentrated under reduced pressure. The crude product was purified by prep-HPLC to afford 2-phenoxy-6,7,8,9-tetrahydro-5H-pyrazino[2,3-d]azepine as a yellow solid. LCMS (ESI): [M+H]⁺=242.

The compounds in Table 3 were prepared in a similar manner.

TABLE 3

| Ex- | | |
|---|---|---|
| am- | | |
| ple | Compound Structure | Product Mass |
| 26 | 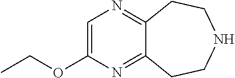 | LCMS [M + H]⁺ 194 |
| 29 | 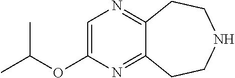 | LCMS [M + H]⁺ 208 |
| 25 | 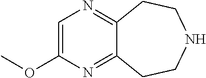 | LCMS [M + H]⁺ 180 |
| 27 | 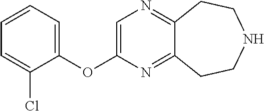 [SMILES: Clc1ccccc1Oc1cnc2c(n1)CCNCC2] | LCMS [M + H]⁺ 277 |
| 18 | 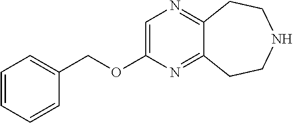 [SMILES: c1ccc(COc2cnc3c(n2)CCNCC3)cc1] | LCMS [M + H]⁺ 256 |
| 30 | 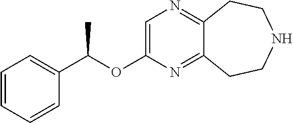 [SMILES: C[C@@H](Oc1cnc2c(n1)CCNCC2)c1ccccc1] | LCMS [M + H]⁺ 270 |
| 31 | 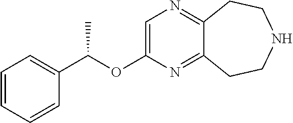 [SMILES: C[C@H](Oc1cnc2c(n1)CCNCC2)c1ccccc1] | LCMS [M + H]⁺ 270 |

### Example 19: Synthesis of 6,7,8,9-tetrahydro-5H-pyrazino2,3-dazepin-2-amine

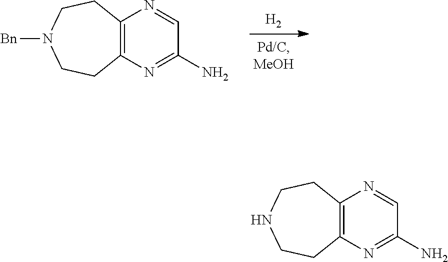 [SMILES: Nc1cnc2c(n1)CCN(Cc1ccccc1)CC2 ; "H2" ; Nc1cnc2c(n1)CCNCC2]

To 7-benzyl-6,7,8,9-tetrahydro-5H-pyrazino[2,3-d]azepin-2-amine (1 eq) in MeOH (5 V) was added 10% Pd/C (10 mg) at RT under H₂ balloon pressure for 5 h. After workup and purification gave 20 mg of 6,7,8,9-tetrahydro-5H-pyrazino[2,3-d]azepin-2-amine. LCMS (ESI): [M+H]⁺=164.

The compounds in Table 4 were prepared in a similar manner.

TABLE 4

| Example | Compound Structure | Product Mass |
|---|---|---|
| 20 | 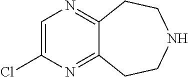 [SMILES: Clc1cnc2c(n1)CCNCC2] | LCMS [M + H]⁺ 184 |
| 23 | 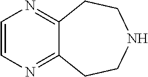 [SMILES: c1cnc2c(n1)CCNCC2] | LCMS [M + H]⁺ 150 |

### Synthesis of 2-(difluoromethoxy)-6,7,8,9-tetrahydro-5H-pyrazino[2,3-d]azepine (Example 28)

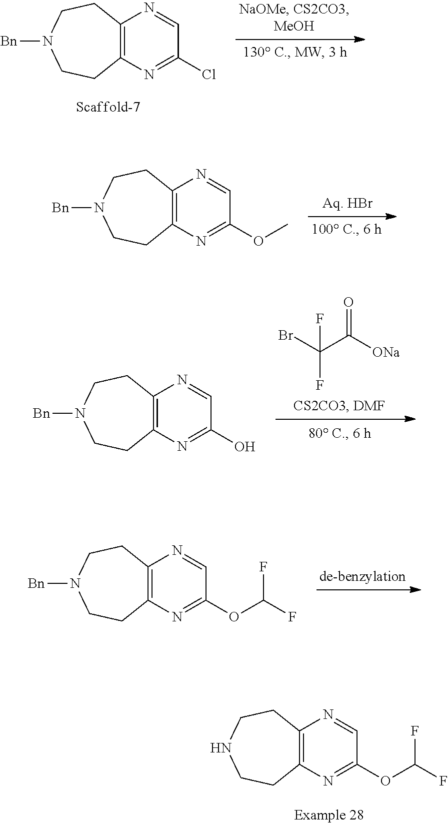

To 7-benzyl-2-chloro-5H,6H,8H,9H-pyrazino[2,3-d]azepine (1 eq) in MeOH (10 V) was added NaOMe (3 eq), Cₛ₂CO₃ (3 eq) at room temperature to 130° C. in microwave for 2.5 h. After workup and purification, 120 mg of 7-benzyl-2-methoxy-6,7,8,9-tetrahydro-5H-pyrazino[2,3-d]azepine (LCMS [M+H]⁺ 270) was obtained. TLC was matches with authentic spot. 7-Benzyl-2-methoxy-6,7,8,9-tetrahydro-5H-pyrazino[2,3-d]azepine (1 eq) was treated with Aqueous HBr (10 V) at RT to 100° C. for 6 h. After workup, 70 mg of crude compound 7-benzyl-6,7,8,9-tetrahydro-5H-pyrazino[2,3-d]azepin-2-ol (LCMS [M+H]⁺ 256) was obtained and used directly in the next step without further purification.

To 7-benzyl-6,7,8,9-tetrahydro-5H-pyrazino[2,3-d]azepin-2-ol (1 eq) in DMF(10 V) was then added Cₛ₂CO₃ (3 eq) followed by sodium bromodifluoroacetate (3 eq) at RT. The mixture was heated at 80° C. for 6 h. After workup, 80 mg of crude 7-benzyl-2-(difluoromethoxy)-6,7,8,9-tetrahydro-5H-pyrazino[2,3-d]azepine (LCMS [M+H]⁺ 306).

To crude 7-benzyl-2-(difluoromethoxy)-6,7,8,9-tetrahydro-5H-pyrazino[2,3-d]azepine (1 eq) in toluene (10 V) was then added DIPEA (8 eq) followed by 1-chloro ethylchloroformate(8 eq) at room temperature. The mixture was heated at 100° C. for 2 h. After 2 h, solvent was removed under reduced pressure. MeOH (0.5 mL) was added, and the reaction mixture was heated at 80° C. for 1 h. After workup and purification, 6 mg (11% yield) of 2-(difluoromethoxy)-6,7,8,9-tetrahydro-5H-pyrazino[2,3-d]azepine (LCMS (ESI): [M+H]⁺=216)

### Example 9: Synthesis of 3-methoxy-8-methyl-5,6,7,8,9,10-hexahydropyrazino-[2′,3′:4,5]pyrrolo[2,3-d]azepine

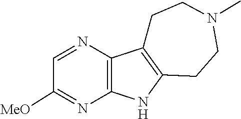 [SMILES: COc1cnc2c3c([nH]c2n1)CCN(C)CC3]

To a solution of 2-hydrazinyl-6-methoxypyrazine (1.0 mmol) in EtOH (0.1 M) was added 1-methylazepan-4-one hydrochloride (164 mg, 1.0 mmol, 1.0 equiv) followed by concentrated aqueous HCl (0.5 mL, 6.0 mmol, 6.0 equiv). The mixture was refluxed for 24 h and then concentrated under reduced pressure. The oily residue was dissolved in DCM (˜25 mL) and basified with 1 M aqueous NaOH (˜20 mL). The aqueous layer was extracted with DCM (3×20 mL). The combined organic extracts were dried over Na₂SO₄ and concentrated under reduced pressure to yield an oil that was purified by chromatography on silica gel (20:1 DCM:MeOH with 0.5% NH₄OH) to yield 3-methoxy-8-methyl-5,6,7,8,9,10-hexahydropyrazino-[2′,3′:4,5]pyrrolo[2,3-d]azepine (LCMS: [M+H]⁺ 265).

The compounds in Table 5 were prepared in a similar manner.

TABLE 5

| Example | Compound Structure | Product Mass |
|---|---|---|
| 10 | 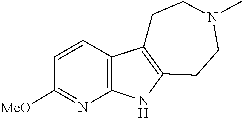 [SMILES: COc1ccc2c3c([nH]c2n1)CCN(C)CC3] | LCMS [M + H]⁺ 232 |
| 11 | 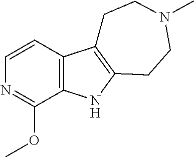 [SMILES: COc1nccc2c3c([nH]c12)CCN(C)CC3] | LCMS [M + H ]⁺ 232 |

### Example 12: Synthesis of 3-methoxy-5-methyl-5,6,7,8,9,10-hexahydropyrazino[2′,3′:4,5]-pyrrolo[2,3-d]azepine

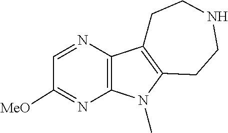 [SMILES: COc1cnc2c3c(n(C)c2n1)CCNCC3]

Step 1: To a solution of 2-hydrazinyl-6-methoxypyrazine (1.0 mmol) in EtOH (0.1 M) was added 1-benzylazepan-4-one hydrochloride (1.0 mmol, 1.0 equiv) followed by concentrated aqueous HCl (0.5 mL, 6.0 mmol, 6.0 equiv). The mixture was refluxed for 24 h and then concentrated under reduced pressure. The oily residue was dissolved in DCM (˜25 mL) and basified with 1 M aqueous NaOH (˜20 mL). The aqueous layer was extracted with DCM (3×20 mL). The combined organic extracts were dried over Na₂SO₄ and concentrated under reduced pressure to yield an oil that was purified by chromatography on silica gel (20:1 DCM:MeOH with 0.5% NH₄OH).

Step 2: To a solution of the benzyl intermediate in THF cooled to 0° C. was added NaH (60% dispersion in oil, 1.5 eqv). The mixture was allowed to warm to room temperature and stirred for another 30 min. The mixture was cooled and methyl iodide (1.5 eqv) was added and the mixture was stired for an additional 1 h. The mixture was quenched with a few drops of methanol, diluted with DCM then washed with brine. The combined organic extracts were dried over Na₂SO₄ and concentrated under reduced pressure to yield an oil that was purified by chromatography on silica gel (20:1 DCM:MeOH with 0.5% NH₄OH).

Step 3: To a solution of the methylated benzyl intermediate in methanol was added 10% palladium on carbon. The mixture was then treated with hydrogen gas at room temperature and stirred for overnight. The mixture was filted to a pad of celite and the filtrate was concentrated in vacuo to give the title compound.

### Example 13: 1,7-dimethyl-5,6,7,8,9,10-hexahydropyrido[3′,2′:4,5]pyrrolo[2,3-d]azepin-2(1H)-one

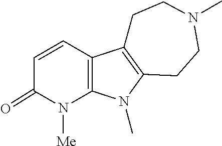 [SMILES: CN1CCc2c(n(C)c3c2ccc(=O)n3C)CC1]

Step 1: To a solution of Example 10 (1.0 mmol) in DCM (0.1 M) was added Tosyl chloride (1.0 mmol, 1.5 equiv) followed by Hunigs base (6.0 equiv). The mixture was stirred for 24 h and then concentrated under reduced pressure. The oily residue was dissolved in DCM (˜25 mL) and basified washed with brine. The aqueous layer was extracted with DCM (3×20 mL). The combined organic extracts were dried over Na₂SO₄ and concentrated under reduced pressure to yield an oil that was directly in the next step.

Step 2: To a solution of the Tosylated intermediate (1.0 mmol) in DCM cooled to −78° C. was added BBr3 in THF. The mixture was allowed to warm to room temperature and stirred for another 2 h. The mixture was quenched with a few drops of methanol, diluted with DCM then washed with brine. The combined organic extracts were dried over Na₂SO₄ and concentrated under reduced pressure to yield the title NH-pyridone.

Step 3: Treatment a solution of the Tosylated pyridone (1.0 mmol) in DMF was added Cₛ₂CO₃ followed by Mel. The mixture was allowed to warm to room temperature and stirred for another 2 h. The mixture was quenched with a few drops of methanol, the treated NaOH to remove the Tosyl group. The mixture was diluted with DCM then washed with brine. The combined organic extracts were dried over Na₂SO₄ and concentrated under reduced pressure to yield the title methylpyridone, Example 13.

The compounds in Table 6 were prepared in a similar manner.

TABLE 6

| Example | Compound Structure | Product Mass |
|---|---|---|
| 14 | 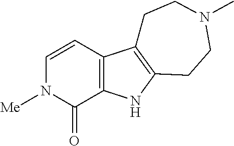 [SMILES: CN1CCc2[nH]c3c(=O)n(C)ccc3c2CC1] | LCMS [M + H]⁺ 232 |

### Example 22: Synthesis of 7-methyl-5,6,7,8,9,10-hexahydropyrido[3′,2′:4,5]pyrrolo[2,3-d]azepine

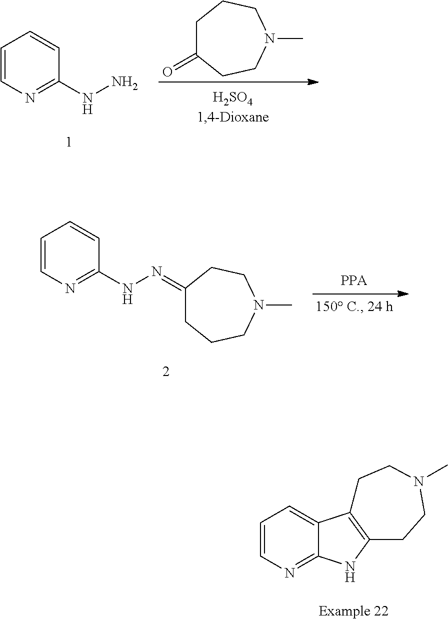

A solution of 2-hydrazineylpyridine (0.4 g, 3.66 mmol) in 1,4-dioxane (20 mL) was treated with H₂SO₄ (0.5 mL) followed by 1-methylazepan-4-one hydrochloride (0.72 g, 4.39 mmol) at room temperature and was stirred at 80° C. for 30 min. The reaction was brought to room temperature, basified with 4 N NaOH solution and product was extracted into ethyl acetate (2×25 mL). Combined ethyl acetate layer was dried (Na₂SO₄) and solvent was evaporated to obtain crude 1-methyl-4-(2-(pyridin-2-yl)hydrazineylidene)azepane intermediate as brown oil.

Above crude intermediate was treated with PPA (30 g) and the reaction was stirred at 150° C. for overnight (18 h). The reaction was brought to 80° C., carefully quenched with water. The reaction was brought to room temperature and basified with 4 N NaOH and diluted with water (1 L). Product was extracted into CH₂Cl₂ (3×50 mL) and combined CH₂Cl₂ layer was dried (Na₂SO₄). Solvent was evaporated and crude was purified by column chromatography (2 M NH₃ in MeOH: CH₂Cl₂, 5:95) on silica gel to obtain 7-methyl-5,6,7,8,9,10-hexahydropyrido[3′,2′:4,5]pyrrolo[2,3-d]azepine (0.187 g, 25.4%) as alight brown solid. ¹HNMR (DMSO-d₆): δ 2.41 (s, 3H), 2.70-2.82 (m, 6H), 2.91-2.94 (m, 2H), 6.97 (dd, 1H, J=3.0, 6.0 Hz), 7.78 (dd, 1H, J=3.0, 6.0 Hz), 8.07 (d, 1H, J=3.0 Hz), 11.24 (s, 1H); LCMS (ESI-MS): 202 (MH⁺).

II. Biological Evaluation

5-HT2 Receptor Assays

Compounds of the present application bind to the 5HT2 receptor subtypes in the following assays: Compounds of the invention were tested on 5-HT2B, 5-HT2A, 5HT2C human recombinant G protein-coupled receptors using a CHO-K1-mt aequorin Gα16 cell line and IP-One assays (Euroscreen Laboratory, Belgium). Dose-response curves for the test compounds are generated over the concentration range of 0.01 to 20,000 nM to determine effective concentration (EC₅₀), inhibitory concentration (IC₅₀) and relative degree of agonistic and antagonistic response (“relative response”). Preferably the compounds of the present application bind to the 5-HT_{2A} and/or 5HT2C receptor. Preferably the compounds of the present application do not bind, or minimally bind, to the 5-HT2B receptor.

Procedure for 5-HT2A, 5-HT2B and 5-HT2C Pharmacological Screening by FLIPR Assay in Agonist mode
1. Culture the cells in cell culture medium (DMEM containing 10% dialyzed FBS, 1×penicillin-streptomycin, 100 μg/ml hygromycin B and 300 μg/ml G418) at 37° C., 5% (v/v) CO₂.
2. One day before the assays, detach the cell using TrypLE™ Express and count cells using cell counter. Only cells with >85% viability are used for the assay.
3. Seed 20000 cells/well in 30 μL/well culture medium to a 384-well cell plate and incubate the cells overnight at 37° C., 5% (v/v) CO₂.
4. On the assay day, prepare 2×dye solution following the manual of the FLIPR® Calcium 6

Assay Kit:
i. Dilute the dye with assay buffer (20 mM HEPES in 1×HBSS, PH7.4);
ii. Add probenecid to the final concentration of 5 mM.
iii. Vortex vigorously for 1-2 minutes.
5. Remove medium from cell plate by flicking the cell plate on towel papers.
6. Add 10 μL of assay buffer and 10 μL of 2×dye solution to each well of the cell plate.
7. Put the cell plate on plate shaker, agitate the plate at 600 rpm for 2 minutes. Incubate the plate at 37° C. for 2 hours followed by additional 15-minute incubation at 25° C.
8. Prepare 3×compound in assay buffer:
   a. Dilute reference compounds to required concentration with DMSO. Add the compounds to a 384-well compound plate.
   b. Perform serial dilutions.
   c. Add 30 mM test compounds to the compound plate, perform 3-fold serial dilutions.
   d. Transfer 90 nL/well of reference compounds and test compounds from source plate to a 384-well compound plate by using an Echo.
   e. Add 30 μL/well assay buffer to the compound plate.
   f. Mix the plate-on-plate shaker for 2 mins.
9. Put the cell plate, compound plate and tips into FLIPR, transfer 10 μL of 3×compound to the cell plate per well with FLIPR.
10. Read the plate for 160 sec with 1 sec interval to obtain the data of agonist mode.

The normalized fluorescence reading (RFU) is calculated as shown follow, while Fmnax and Fmin stand for maximum and minimum of calcium signal during defined time window:

$$\\ RFU = F max - F min$$

$$\\ \% Activation = \frac{( RFUcompound - RFU low control )}{( \begin{matrix}RFU top concentration of reference agonist - \\ RFU low control\end{matrix} )} * 100 \%$$

11. Calculate EC₅₀ by filling % activation against log of compound concentrations with Hill equation using XLfit.

| | | 5-HT2A | 5-HT2B | 5-HT2C |
|---|---|---|---|---|
| | | (EC₅₀, % | (EC₅₀, % | (EC₅₀, % |
| Example | | max | max | max |
| Number | Structure | activity) | activity) | activity |
| Serotonin | | 7 | 1 | 7 |
| 1 | | C | D | A |
| 2 | | B | C | A |
| 4 | | C | B | n.t. |
| 10 | 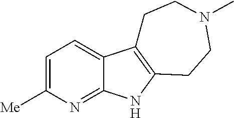 [SMILES: Cc1ccc2c3c([nH]c2n1)CCN(C)CC3] | D | D | D |
| 15 | 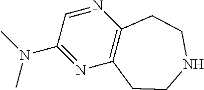 | D | B | n.t. |
| 16 | 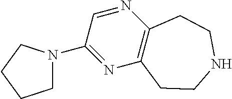 [SMILES: c1nc2c(nc1N1CCCC1)CCNCC2] | D | B | n.t. |
| 17 | 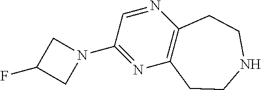 [SMILES: FC1CN(c2cnc3c(n2)CCNCC3)C1] | D | D | n.t. |
| 18 | 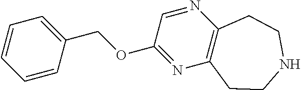 [SMILES: c1ccc(COc2cnc3c(n2)CCNCC3)cc1] | D | A | n.t. |
| 19 | 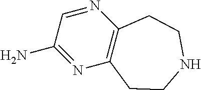 [SMILES: Nc1cnc2c(n1)CCNCC2] | D | D | n.t. |
| 20 | 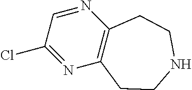 [SMILES: Clc1cnc2c(n1)CCNCC2] | D | D | n.t. |
| 22 | 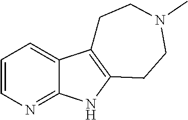 | D | D | D |

Legend: <200 = A; 200-1000 = B; 1000-6000 = C; >6000 = D; n.t. = testing in progress

The 5-HT2A, (EC50, % max activity), the 5-HT2B, (EC50, % max activity), and the 5-HT2C, (EC50, % max activity) values for example 1, which has the structure

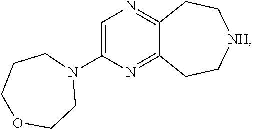 [SMILES: c1nc2c(nc1N1CCCOCC1)CCNCC2]

were about 2000, greater than about 30000, and about 100, respectively.

The 5-HT2A, (EC50, % max activity), the 5-HT2B, (EC50, % max activity), and The 5-HT2C,

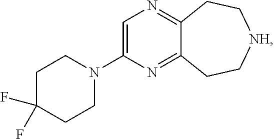 [SMILES: FC1(F)CCN(c2cnc3c(n2)CCNCC3)CC1]

(EC50, % max activity) values for example 2, which has the structure
were about 900, about 4000, and about 200, respectively.

Microsomal Stability Assays

Liver Microsomal Metabolic Stability

In Phase I analysis test compounds are incubated at a final concentration of 1 μM (this concentration is assumed to be well below the Km values to ensure linear reaction conditions i.e. to avoid saturation). Working stocks are initially diluted to a concentration of 40.0 μM in 0.1 M potassium phosphate buffer (pH 7.4) before addition to the reaction vials. CD-1 mouse (male) or pooled human liver microsomes (Coming Gentest) are utilized at a final concentration of 0.5 mg/mL (protein). Duplicate wells are used for each time point (0 and 60 minutes). Reactions are carried out at 37° C. in an orbital shaker at 175 rpm, and the final DMSO concentration is kept constant at 0.1%. The final volume for each reaction is 100 μL, which includes the addition of an NADPH-Regeneration Solution (NRS) mix. This NRS mix is comprised of glucose 6-phosphate dehydrogenase, NADP+, MgCl2, and glucose 6-phosphate. Upon completion of the 60 minute time point, reactions are terminated by the addition of 2-volumes (200 μL) of ice-cold, acetonitrile containing 0.5% formic acid and internal standard. Samples are then centrifuged at 4,000 rpm for 10 minutes to remove debris and precipitated protein. Approximately 150 μL of supernatant is subsequently transferred to a new 96 well microplate for LC/MS analysis:

Narrow-window mass extraction LC-MS analysis is performed for all samples in this study using a Waters Xevo quadrupole time-of-flight (QTof) mass spectrometer to determine relative peak areas of test compounds. The percent remaining values are calculated using the following equations:

% remaining=(A)/A0×100
where
A is area response after incubation
A0 is area response at initial time point

For intrinsic clearance assay, incubation mixtures contain probe substrate, liver microsomes and an NADPH regenerating system (1.3 mM NADP+, 3.3 mM glucose 6-phosphate, 0.4 U ml-1 glucose 6-phosphate dehydrogenase, 3.3 mM magnesium chloride) in 0.1 M potassium phosphate buffer (pH 7.4). CD-1 mouse (male) or pooled human liver microsomes (Coming Gentest) are utilized at a final concentration of 0.5 mg/mL (protein). 12.5 μL of each drug solution are placed into a well of 96 well plate. Reactions are initiated by the addition of activated microsome solutions (500 μL) to drug solutions. Reactions are carried out at 37° C. in an orbital shaker at 175 rpm, and the final DMSO concentration is kept constant at 0.1%. Test compounds are incubated at a final concentration of 1 μM. 50 μL of aliquots of reaction mixtures are quenched by mixing with two parts of stop solution (internal standard containing 0.5% formic acid in acetonitrile) at appropriate time-points and mixed well. Then, solutions are centrifuged at 4000 rpm for 10 min. Supernatants are transferred to a new 96-well plate and analyzed by a Waters Q-TOF mass spectrometer coupled with an UPLC System. Recovery analysis is performed using relative peak areas and narrow window mass extraction.

The ln(% remaining) is plotted against time and the gradient of the line determined.

Elimination Constant (k)=−slope

Half-life (t½)(min)=ln2/k=0.693/k

V(μL/mg)=volume of incubation (μL)/protein in the incubation (mg)

Intrinsic Clearance (CLint)(μL/min/mg protein)=V·0.693/t½=V·k

III. Preparation of Pharmaceutical Dosage Forms

### Example 1: Oral Capsule

The active ingredient is a compound of Table 1, or a pharmaceutically acceptable salt or solvate thereof. A capsule for oral administration is prepared by mixing 1-1000 mg of active ingredient with starch or other suitable powder blend. The mixture is incorporated into an oral dosage unit such as a hard gelatin capsule, which is suitable for oral administration.

### Example 2: Solution for Injection

The active ingredient is a compound of Table 1, or a pharmaceutically acceptable salt thereof, and can be formulated as a solution in sesame oil at a concentration of 50 mg-eq/mL.

The examples and embodiments described herein are for illustrative purposes only and various modifications or changes suggested to persons skilled in the art are to be included within the spirit and purview of this application and scope of the appended claims.

1-153. (canceled)

154. A compound, or pharmaceutically acceptable salt or solvate thereof, having the structure of Formula (I): 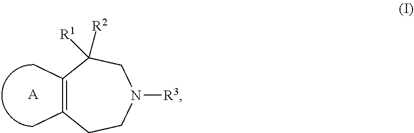
wherein
R¹, R² and R³ are each independently selected from H and C₁-C₆ optionally substituted alkyl; and
ring A is an optionally substituted heteroaryl ring selected from 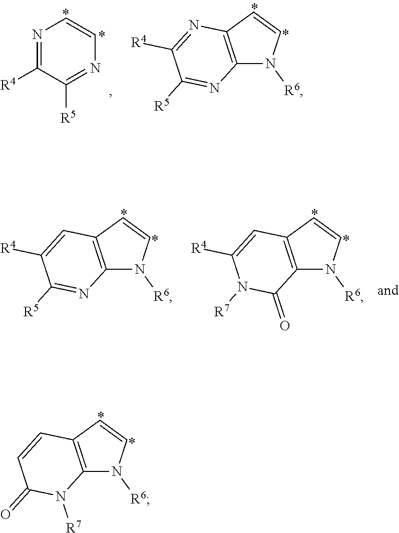
wherein
* represents the points of attachment;
R⁴ and R⁵ are independently selected from H, halo, CN, C₁₋₄alkyl, C₁₋₄haloalkyl, OC₁₋₄alkyl, OC₁₋₄haloalkyl, O(optionally substituted C₃₋₁₀ carbocycle),
O(optionally substituted C₃₋₁₀ aryl), and N(R⁸R⁹);
R⁶ and R⁷ are independently selected from H and C₁-C₆ optionally substituted alkyl; and
R⁸ and R⁹ are joined to form, together with the atom therebetween, a C₃₋₁₂heterocycloalkyl, optionally comprising one additional heteromoiety selected from N(R¹⁰), O, S, S(O) and SO₂, and optionally substituted with one or more substituents selected from halo, OH, C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), N(C₁₋₆alkyl)(C₁₋₆alkyl), SC₁₋₆alkyl, S(O)C₁₋₆alkyl, and SO₂C₁₋₆alkyl, wherein
R¹⁰ is selected from hydrogen, C₁₋₆alkyl, C₃₋₆cycloalkyl, aryl, C₅-6heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, C(O)NH₂, C(O)NH(C₁₋₆alkyl), C(O)N(C₁₋₆alkyl)(C₁₋₆alkyl), S(O)C₁₋₆alkyl, and SO₂C₁₋₆alkyl; and
all alkyl, alkylene, cycloalkyl, heterocycloalkyl, aryl and heteroaryl groups of the optional substituents on the C₃₋₁₂heterocycloalkyl formed by R⁸ and R⁹ are optionally substituted with one or more substituents selected from halo, C₁₋₆alkyl, OC₁₋₆alkyl, C₁₋₆haloalkyl, and OC₁₋₆haloalkyl.

155. The compound, salt, or solvate of claim 154, wherein ring A is 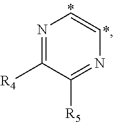
R¹ is H, and R² is C₁₋₆ alkyl.

156. The compound, salt, or solvate of claim 155, wherein ring A is 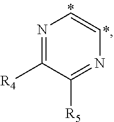
R¹ is H, and R² is methyl.

157. The compound, salt, or solvate of claim 154, wherein ring A is 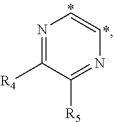
and R³ is H.

158. The compound, salt, or solvate of claim 154, wherein R¹⁰ is selected from hydrogen, and C₁₋₆alkyl.

159. The compound, salt, or solvate of claim 154, wherein the heterocycle formed by R⁸ and R⁹ is selected from

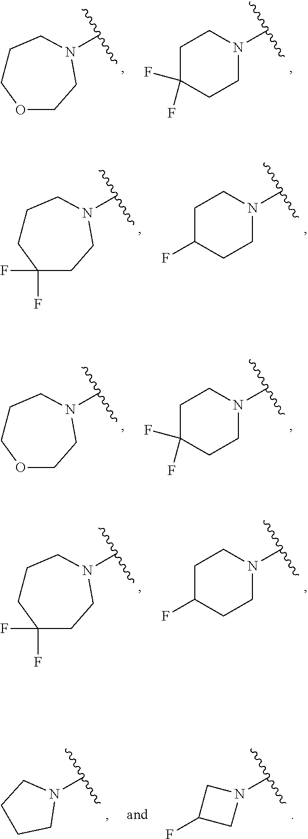

160. The compound, salt, or solvate of claim 154, wherein R⁸ and R⁹ are methyl.

161. The compound, salt, or solvate of claim 154, wherein
R¹ and R² are H;
R³ is selected from H and C₁-C₆ optionally substituted alkyl;
ring A is 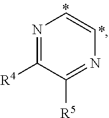
R⁴ and R⁵ are each independently selected from H, C₁₋₄haloalkyl, OC₁₋₄alkyl, OC₁₋₄haloalkyl, O(optionally substituted C₃₋₁₀ carbocycle), O(optionally substituted C₃₋₁₀ aryl), and N(R⁸R⁹);
R⁸ and R⁹ are joined to form, together with the atom therebetween, a C₃₋₁₂heterocycloalkyl, and optionally substituted with one or more substituents selected from halo, OH, NO₂, C₁₋₆alkyl, C₃₋₆cycloalkyl, aryl, C₅₋₆heteroaryl, C₃₋₆ heterocycloalkyl, C₁₋₆alkyleneC₃₋₆cycloalkyl, C₁₋₆alkylenearyl, C₁₋₆alkyleneC₅-6heteroaryl, C₁₋₆alkyleneC₃₋₆heterocycloalkyl, C(O)C₁₋₆alkyl, OC₁₋₆alkyl, OC₁₋₆alkyleneOC₁₋₆alkyl, C(O)NH₂, C(O)NH(C₁₋₆alkyl), C(O)N(C₁₋₆alkyl)(C₁₋₆alkyl), NHC(O)C₁₋₆alkyl, N(C₁₋₆alkyl)C(O)C₁₋₆alkyl, NH₂, NH(C₁₋₆alkyl), N(C₁₋₆alkyl)(C₁₋₆alkyl), SC₁₋₆alkyl, S(O)C₁₋₆alkyl, and SO₂C₁₋₆alkyl, wherein
R¹⁰ is selected from C₁₋₆alkyl, C₃₋₆cycloalkyl, aryl, C₅₋₆heteroaryl, C₃₋₆ heterocycloalkyl, C₁₋₆ alkyleneC₃₋₆cycloalkyl, C₁₋₆alkylenearyl, C₁₋₆alkyleneC₅₋₆heteroaryl, C₁₋₆alkyleneC₃₋₆heterocycloalkyl, C(O)C₁₋₆alkyl, C(O)NH₂, C(O)NH(C₁₋₆alkyl), C(O)N(C₁₋₆alkyl)(C₁₋₆alkyl), S(O)C₁₋₆alkyl, and SO₂C₁₋₆alkyl.

162. The compound, salt, or solvate of claim 159, wherein ring A is 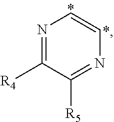
R¹ is H, and R² is C₁₋₆ alkyl.

163. The compound, salt, or solvate of claim 162, wherein ring A is 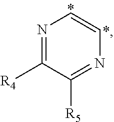
R¹ is H, and R² is methyl.

164. The compound, salt, or solvate of claim 154, wherein R³ is H.

165. The compound, salt, or solvate of claim 164, wherein ring A is 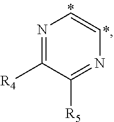
and R³ is H.

166. The compound, salt, or solvate of claim 165, wherein the heterocycle formed by R⁸ and R⁹ is selected from

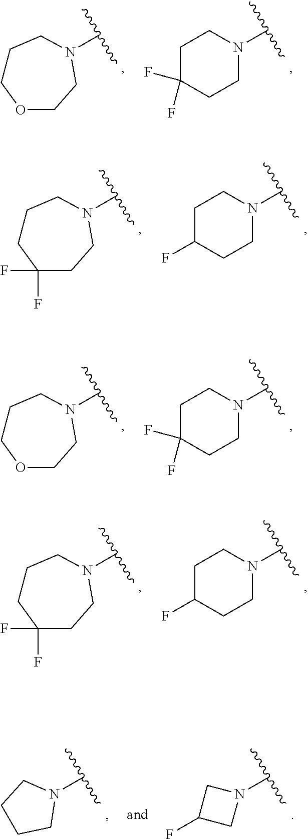

167. The compound, salt, or solvate of claim 165, wherein R⁸ and R⁹ are methyl.

168. The compound of claim 166, or pharmaceutically acceptable salt or solvate thereof, having the structure:

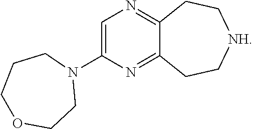 [SMILES: c1nc2c(nc1N1CCCOCC1)CCNCC2]

169. The compound of claim 166, or pharmaceutically acceptable salt or solvate thereof, having the structure:

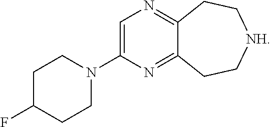 [SMILES: FC1CCN(c2cnc3c(n2)CCNCC3)CC1]

170. The compound of claim 166, or pharmaceutically acceptable salt or solvate thereof, having the structure:

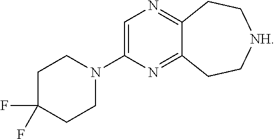 [SMILES: FC1(F)CCN(c2cnc3c(n2)CCNCC3)CC1]

171. The compound of claim 166, or pharmaceutically acceptable salt or solvate thereof, having the structure:

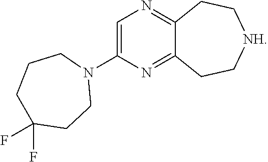 [SMILES: FC1(F)CCCN(c2cnc3c(n2)CCNCC3)CC1]

172. A compound, or pharmaceutically acceptable salt or solvate thereof, as provided in Table 1.

173. A compound, or pharmaceutically acceptable salt or solvate thereof, selected from 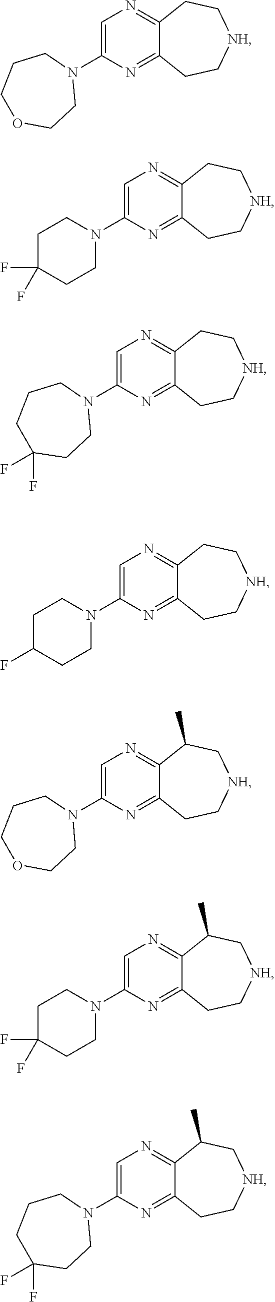 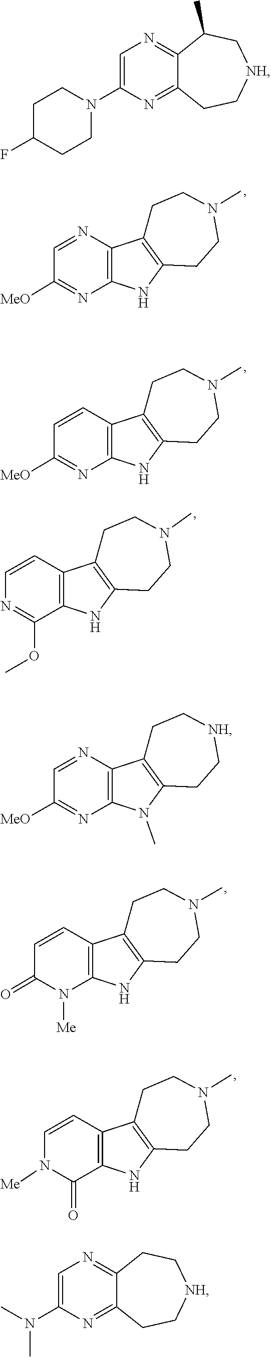 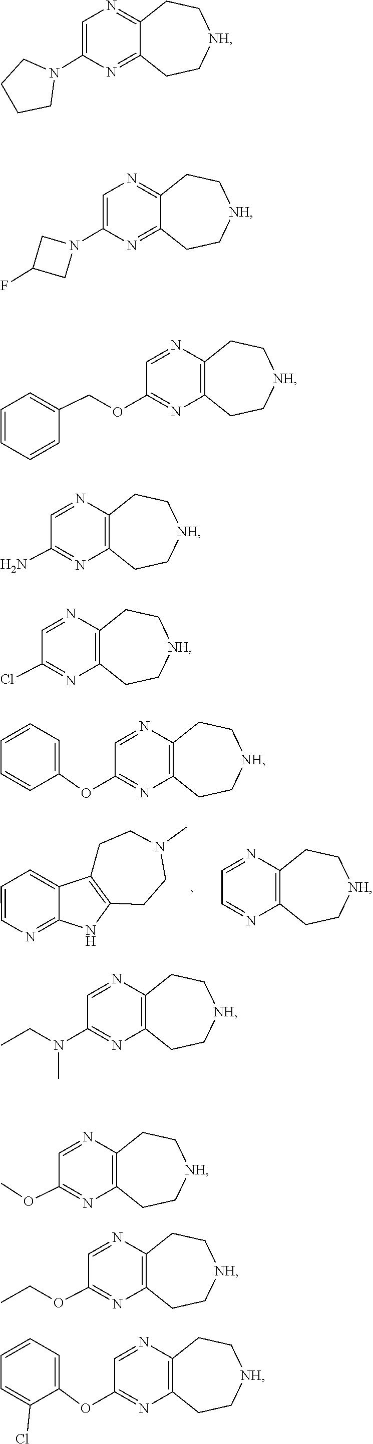 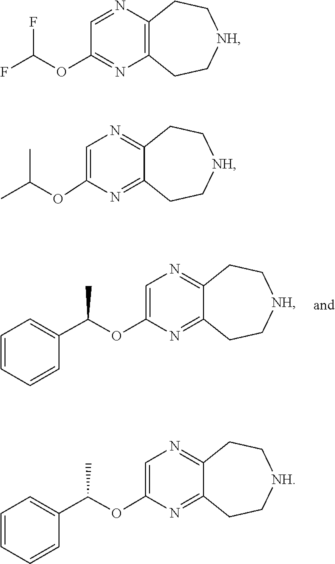 [SMILES: FC(F)Oc1cnc2c(n1)CCNCC2 ; CC(C)Oc1cnc2c(n1)CCNCC2 ; C[C@@H](Oc1cnc2c(n1)CCNCC2)c1ccccc1 ; C[C@H](Oc1cnc2c(n1)CCNCC2)c1ccccc1]
